(19) United States
(12) Patent Application Publication
(10) Pub. No.: US 2026/0053954 A1
(43) Pub. Date: Feb. 26, 2026

(54) Benzaldehyde acetal acid-degradable amphiphilic lipid and self-assembling peptides

(71) Applicant: The Regents of the University of California, Oakland, CA (US)

(72) Inventors: Niren Murthy, Berkeley, CA (US); Hesong Han, Berkeley, CA (US); Sheng Zhao, Berkeley, CA (US)

(73) Assignee: The Regents of the University of California, Oakland, CA (US)

(21) Appl. No.: 19/308,284

(22) Filed: Aug. 24, 2025

Related U.S. Application Data
(63) Continuation of application No. PCT/US24/19255, filed on Mar. 8, 2024.
(60) Provisional application No. 63/489,179, filed on Mar. 8, 2023.
(60) Provisional application No. 63/489,181, filed on Mar. 8, 2023.

(51) Int. Cl. A61K 48/00

(52) U.S. Cl. CPC A61K 48/0058; A61K 48/0041; A61K 48/0091

(57) ABSTRACT

Compounds comprising a benzaldehyde acetal acid-degradable amphiphilic lipid and self-assembling peptides are incorporated in lipid nanoparticle (LNP) and used to transfect cells.

### CROSS-REFERENCES TO RELATED APPLICATIONS

This application is a continuation of PCT/US24/19255; filed Mar. 8, 2024, which claims priority to U.S. Provisional Application No. 63/489,179; filed Mar. 8, 2023 and to U.S. Provisional Application No. 63/489,181; filed Mar. 8, 2023, the disclosures of which are hereby incorporated by reference in its entirety for all purposes.

### REFERENCE TO A SEQUENCE LISTING

A Sequence Listing in XML format is incorporated by reference into the specification. The name of the XML file containing the Sequence Listing is B23-059-4US.xml. The XML file is 63,916 bytes and was created on Aug. 24, 2025.

### INTRODUCTION

Solid lipid nanoparticles are useful for delivering mRNA, and have potential to treat a wide variety of diseases. SLNs contain a PEGylated lipid, which is generally in the 1-5% range and is needed to maintain SLN stability, size, tissue diffusion and lower toxicity. However, excessive PEGylation also results in lower cell uptake and endosomal disruption. This paradox has limited the efficacy of SLNs, and is termed the “PEG dilemma”. Acid degradable PEG-lipids have great potential for overcoming the PEG dilemma, but have been challenging to develop due to the synthetic challenges associated with working with acetals and their instability at pH 7.4.

Relevant literature includes: Knorr et al., Bioconjugate Chem. 2007, 18, 4, 1218-1225; Liu et al., J. Am. Chem. Soc. 2017, 139, 6, 2306-2317

### SUMMARY OF THE INVENTION

The invention provides compounds comprising a benzaldehyde acetal acid-degradable amphiphilic lipid and self-assembling peptides that can be incorporated in lipid nanoparticle (LNP) and used to transfect cells.

In aspects and embodiments, the invention provides:
1. A composition comprising a compound comprising a benzaldehyde acetal acid-degradable amphiphilic lipid and a self-assembling peptide, of structure:

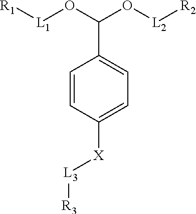

wherein:

R1 comprises a self-assembling peptide or precursor, which can self-assemble after the cleavage of the acid degradable acetal linker, wherein the self-assembled peptide preferably enhances endosome escape;

R2 comprises a hydrophobic group or lipid, such as a steroid (e.g. cholesterol) or one or more alkyl chains, such as in a single chain fatty acid or double or triple chain fatty acid ester, preferably that can strengthen the rigidity of a lipid nanoparticle, and also balance the amphiphilicity of the lipid;

R3 comprises a hydrophilic group, such as a PEG which can stabilize the lipid nanoparticle in serum, and extend the half-life of LNP-mRNA in a physiological environment, and when pairing with the hydrophobic group in the compound, the amphiphilicity of the lipid can be balanced, e.g. by changing the size of PEG;

X is O, S or N; and

L1, L2 and L3 are linkers selected from a bond, an optionally substituted heteroatom and an optionally substituted C1-18 hydrocarbyl or heterohydrocarbyl, providing acid degradable linkages.
2. A composition of claim 1, wherein incorporated in the compound the peptide is in the form of a non-self-assembling precursor that converts to a self-assembling peptide upon intracellular hydrolysis.
3. A composition of claim 1, wherein the peptide is configures so that upon hydrolysis the peptide can form intracellular nanofibrils that disrupt the dynamics of actin filament to cause either apoptosis or necroptosis of the cell, such as cancer cell, see, e.g. Li et al. Adv. Healthcare, Mater. 2017, 6, 1601400.
4. A composition of claim 1, wherein:

R1 comprises 1-10 amino acids, including any combination of D/L.
5. A composition of claim 1, wherein:

R1 comprises a self-assembling peptide selected from:

-NH-F(CO-)
-NH-FF(CO)
-NH-TF(CO)
-NH-YL(CO)
-NH-FFF(CO)
-NH-FFS(CO)
-NH-FFY(CO)
-NH-FFKY(CO-)
(SEQ ID NO: 01)
-NH-FFFY(CO-)
(SEQ ID NO: 02)
-NH-KYFF(CO-)
(SEQ ID NO: 03)
-NH-TFFY(CO-)
(SEQ ID NO: 04)
-NH-FFYY(CO-)
(SEQ ID NO: 05)
-NH-FYFY(CO-)
(SEQ ID NO: 06)
-NH-YFYF(CO-)
(SEQ ID NO: 07)
-NH-FFYE(CO-)
(SEQ ID NO: 08)
-NH-FFYR(CO-)
(SEQ ID NO: 09)
-NH-GFFY(CO-)
(SEQ ID NO: 10)
-NH-LLLY(CO-)
(SEQ ID NO: 11)
-NH-GFYY(CO-)
(SEQ ID NO: 12)
-NH-FFSY(CO-)
(SEQ ID NO: 13)
-NH-FFYS(CO-)
(SEQ ID NO: 14)
-NH-FFAGL(CO-)
(SEQ ID NO: 15)
-NH-FFAG(CO-)
(SEQ ID NO: 16)
-NH-FFYD(CO-)
(SEQ ID NO: 17)
-NH-FFEY(CO-)
(SEQ ID NO: 18)
-NH-LLLLY(CO-)
SEQ ID NO: 19)
-NH-LLYLL(CO-)
(SEQ ID NO: 20)
-NH-FFGGY(CO-)
(SEQ ID NO: 21)
-NH-GFFYSV(CO-)
(SEQ ID NO: 22)
-NH-GFFYG(CO-)
(SEQ ID NO: 23)
-NH-FFGEY(CO-)
(SEQ ID NO: 24)
-NH-FFGDY(CO-)
(SEQ ID NO: 25)
-NH-FFEYI(CO-)
(SEQ ID NO: 26)
-NH-FFFFCG(CO-)
(SEQ ID NO: 27)
-NH-CEYFFG(CO-)
(SEQ ID NO: 28)
-NH-FFYGGAA(CO-)
(SEQ ID NO: 29)
-NH-GFFYGHY(CO-)
(SEQ ID NO: 30)
-NH-FENNQQNY(CO-)
(SEQ ID NO: 31)
-NH-FFAGLDD(CO-)
(SEQ ID NO: 32)
-NH-FFYGGVV(CO-)
SEQ ID NO: 33)
-NH-KRRASVAGK(CO-)
(SEQ ID NO: 34)
-NH-LYYYYL(CO-)
(SEQ ID NO: 35)
-NH-VVVD(CO-)
(SEQ ID NO: 36)
-NH-VVVK(CO-)
(SEQ ID NO: 37)
-NH-VVVVD(CO-)
(SEQ ID NO: 38)
-NH-VVVVDD(CO-)
(SEQ ID NO: 39)
-NH-VVVVWD(CO-)
(SEQ ID NO: 40)
-NH-VVVVWDD(CO-)
(SEQ ID NO: 41)
-NH-IIIIWDD(CO-)
(SEQ ID NO: 42)
-NH-LLLLWDD(CO-)
(SEQ ID NO: 43)
-NH-QQKFQFQFEQQ(CO-)
(SEQ ID NO: 44)
-NH-DID(CO-)
(SEQ ID NO: 45)
-NH-IIIK(CO-)
(SEQ ID NO: 46)
-NH-LLLD(CO-)
(SEQ ID NO: 47)
-NH-LLLK(CO-)
(SEQ ID NO: 48)
-NH-GAVILEE(CO-)
(SEQ ID NO: 49)
-NH-GAVILRR(CO-)
(SEQ ID NO: 50)
-NH-DYKDDDDKG(CO-)
(SEQ ID NO: 51)
-NH-VKVKVKVKVPPTKTEVKVKV(CO-)
(SEQ ID NO: 52)
-NH-AAAAAAD(CO-)
(SEQ ID NO: 53)
-NH-AAAAAAK(CO-)
SEQ ID NO: 54)
-NH-DAAAAAA(CO-)
(SEQ ID NO: 55)
-NH-KAAAAAA(CO-)
(SEQ ID NO: 56)
-NH-YLGFFC(CO-)
(SEQ ID NO: 57)
-NH-KLILIK(CO-)
(SEQ ID NO: 58)
-NH-NYFNTKDRIYH(CO-)
(SEQ ID NO: 59)
-NH-YLGFFC(CO-)
(SEQ ID NO: 60)
-NH-FFFRRR(CO-)
(SEQ ID NO: 61)
-NH-FFFFRRRR(CO-)
(SEQ ID NO: 62)
-NH-FFYSV(CO-)
(SEQ ID NO: 63)
-NH-FFFGKG(CO-)
(SEQ ID NO: 64)
-NH-VVAAEE(CO-)
(SEQ ID NO: 65)
-NH-KLDLKLDLKLDL(CO-)
(SEQ ID NO: 66)
-NH-YGAAKKAAKAAKKAAKAA(CO-)
(SEQ ID NO: 67)
-NH-KFDLKKDLKLDL(CO-)
(SEQ ID NO: 68)
-NH-FKFEFKFF(CO-)
(SEQ ID NO: 69)
-NH-FEFEFKFK(CO-)
(SEQ ID NO: 70).

6. A composition of claim 1, wherein:

R1 comprises a distal hydrophobic moiety (such as pyrene, naphthalene, adamantane, acetate, alkyl or fluorenylmethyloxycarbonate), which may be linked by amide bond on the amino side of the peptide.
7. A composition of claim 1, wherein:

R1 comprises a distal hydrophobic moiety, selected from pyrene, naphthalene, adamantane, acetate, alkyl and fluorenylmethyloxycarbonate, which is linked by amide bond on the amino side of the peptide.
8. A composition of claim 1, wherein:

R1 comprises a distal hydrophobic moiety selected from:

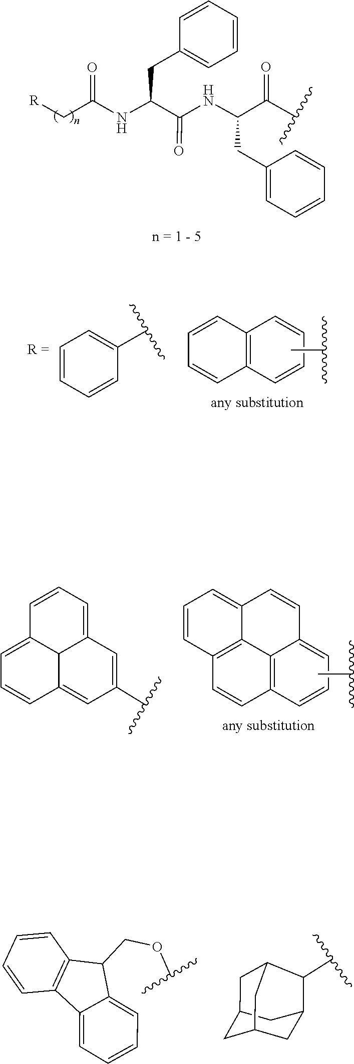

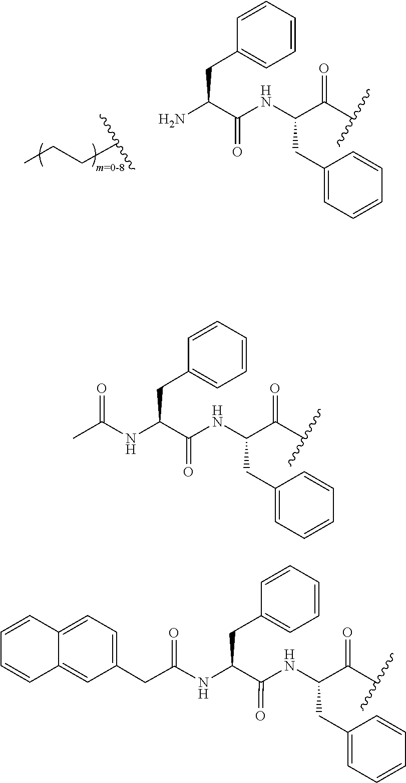

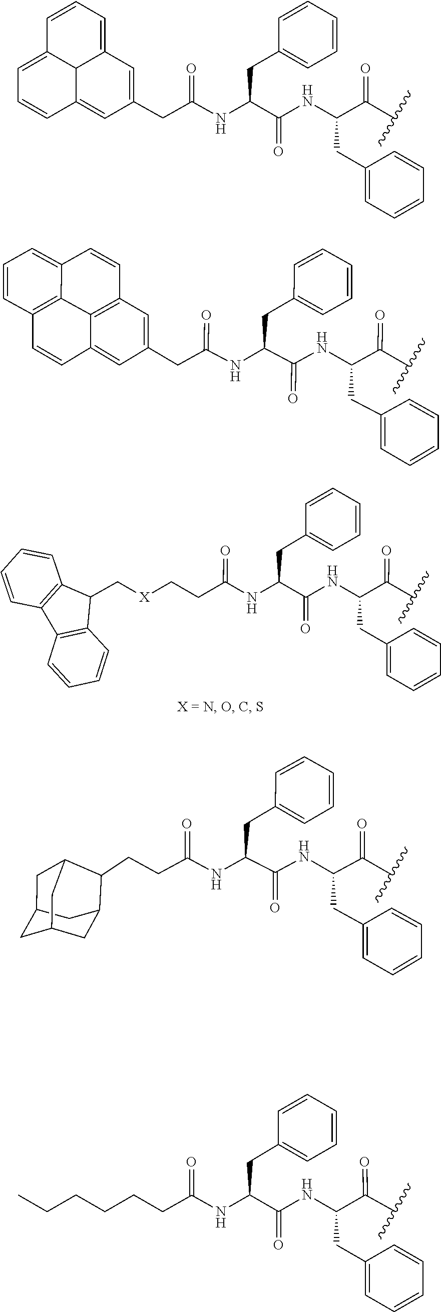

9. A composition of any of claims 1-8, wherein:

R2 comprises steroid selected from:

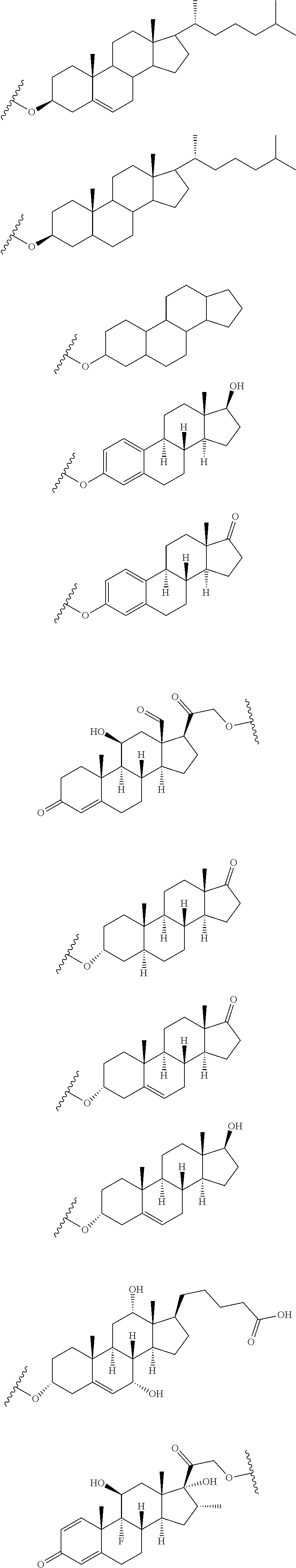

10. A composition of any of claims 1-8, wherein R2 comprises an alkyl chain:

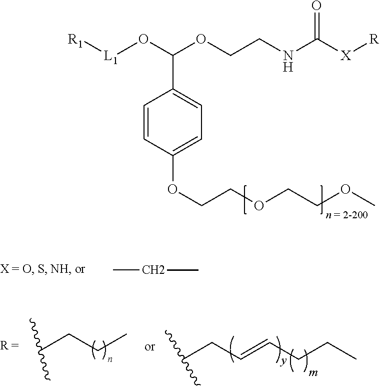

n=4-16; m=1-13; y=1-3, wherein the olefin(s) can be Z/E, and in any position(s) of the chain, such as:

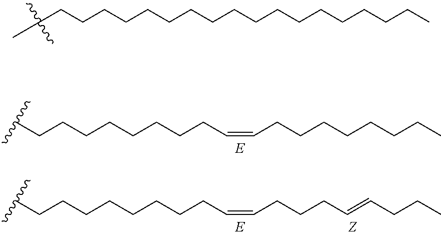 [SMILES: *CCCCCCCCCCCCCCCCCC ; *CCCCCCCC/C=C\CCCCCCCC ; *CCCCCCCC/C=C\CCC/C=C/CCC]

11. A composition of any of claims 1-8, wherein R2 comprises an alkyl chain, of structure:

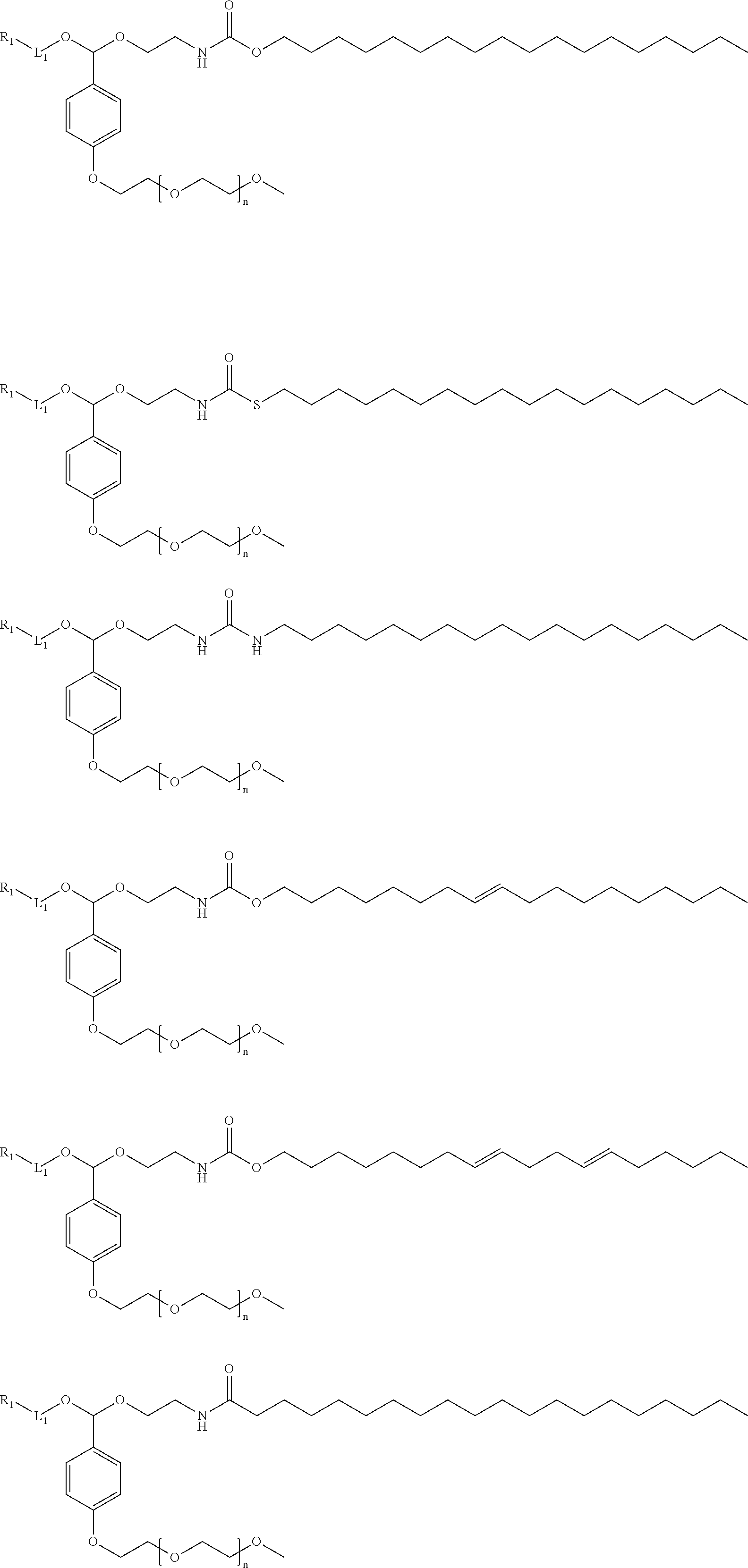

such as in:

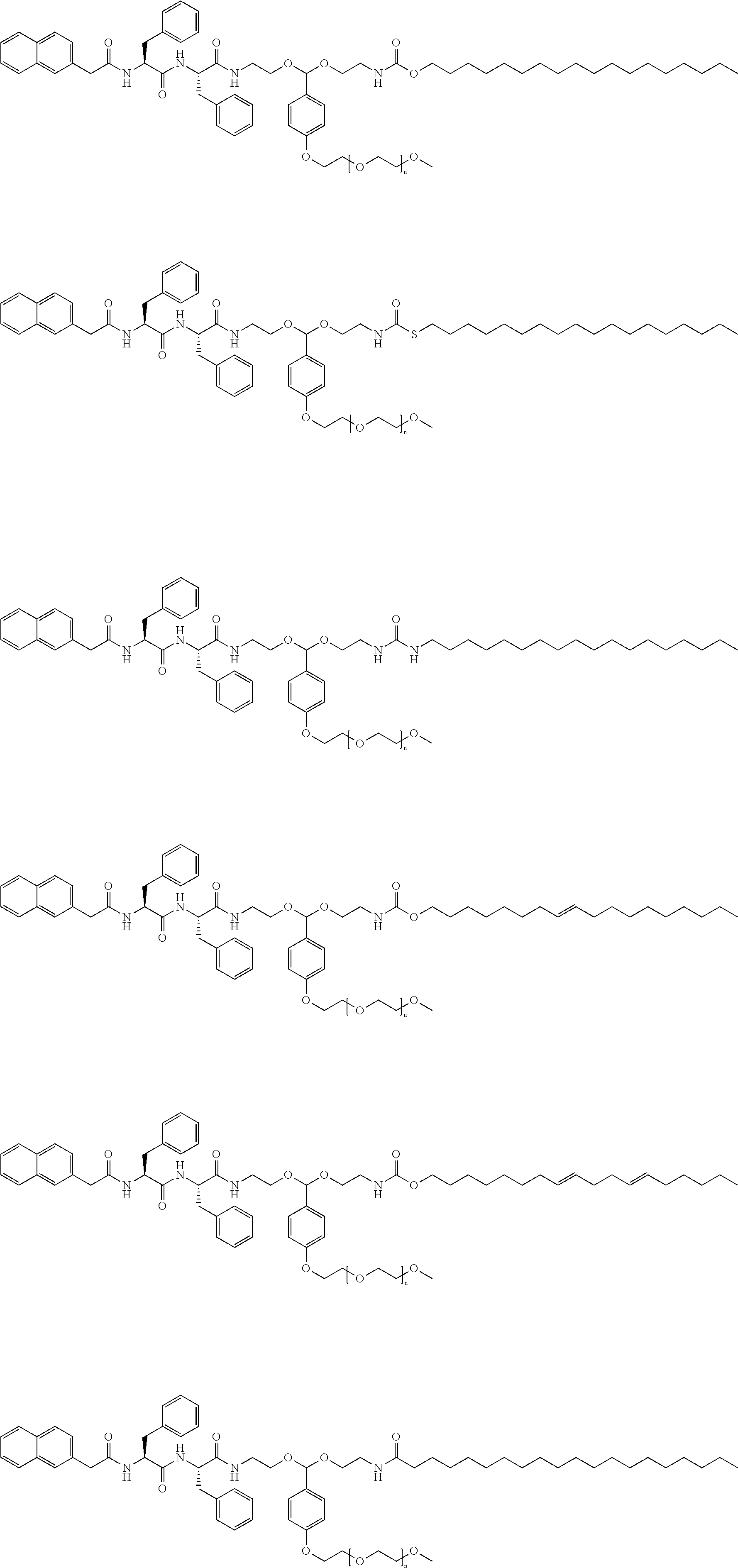

12. A composition of any of claims 1-8, wherein R2 comprises two alkyl chains:

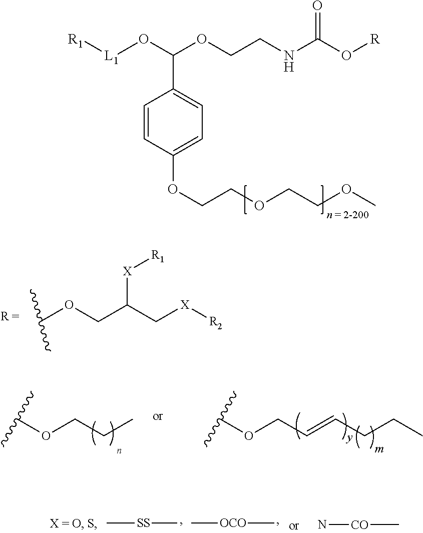

n=4-16; m=1-13; y=1-3, wherein the olefin(s) can be Z/E, and in any position(s) of the chain, such as:

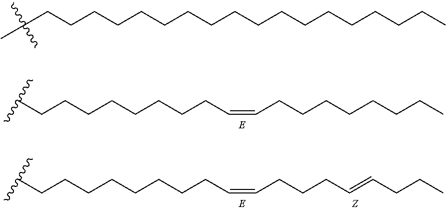 [SMILES: *CCCCCCCCCCCCCCCCCC ; *CCCCCCCC/C=C\CCCCCCCC ; *CCCCCCCC/C=C\CCC/C=C/CCC]

13. A composition of any of claims 1-8, wherein R2 comprises two alkyl chains, of structure:

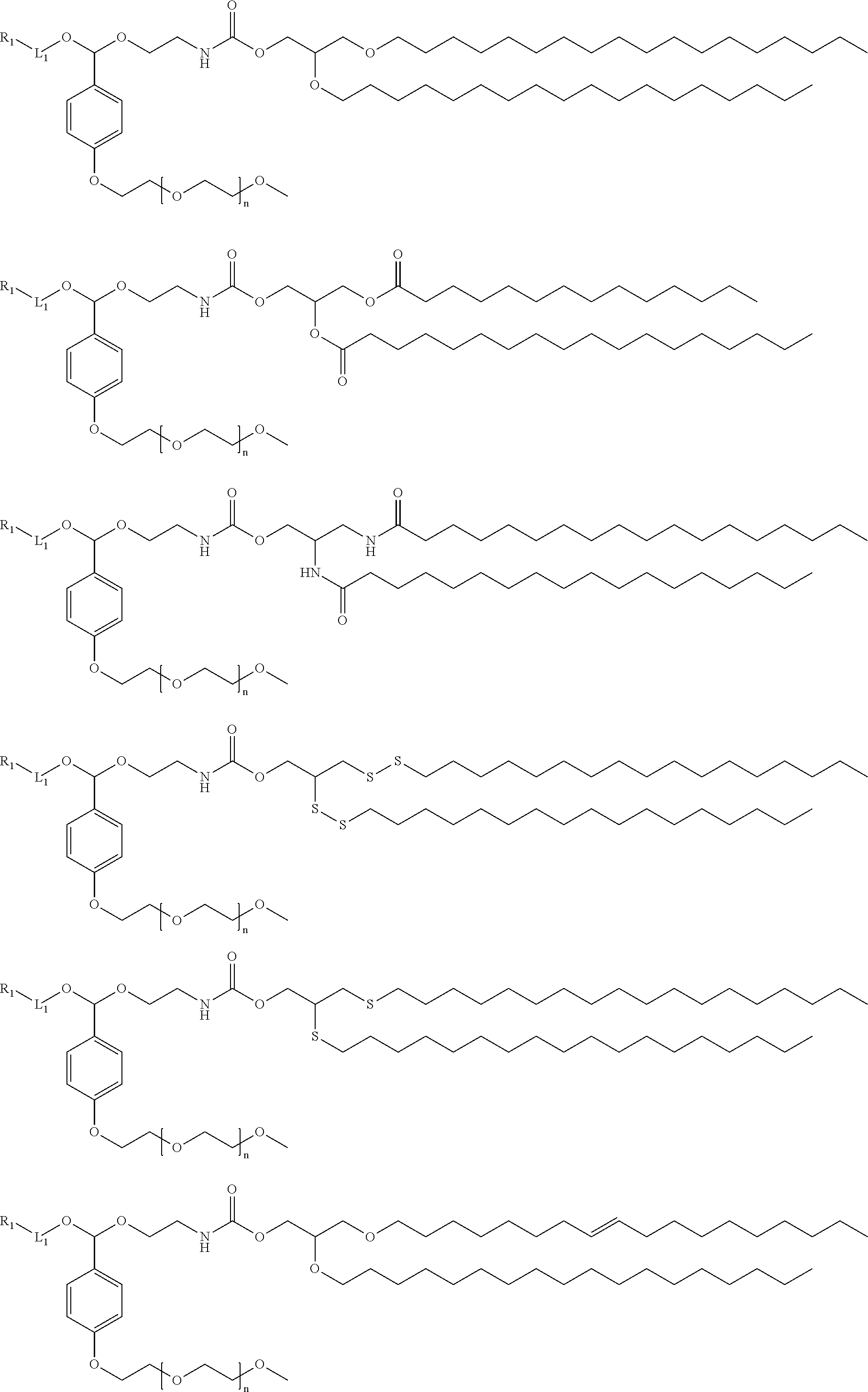

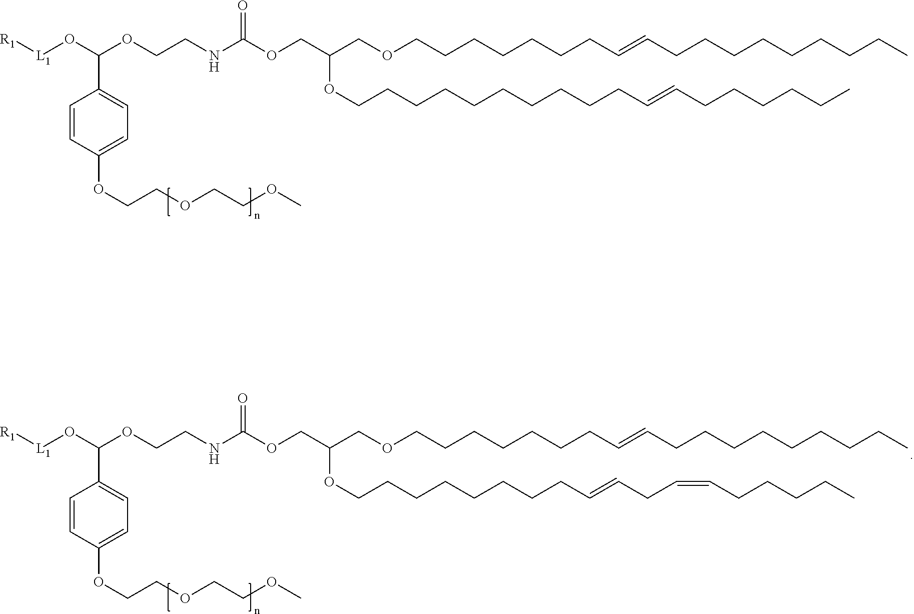

such as in:

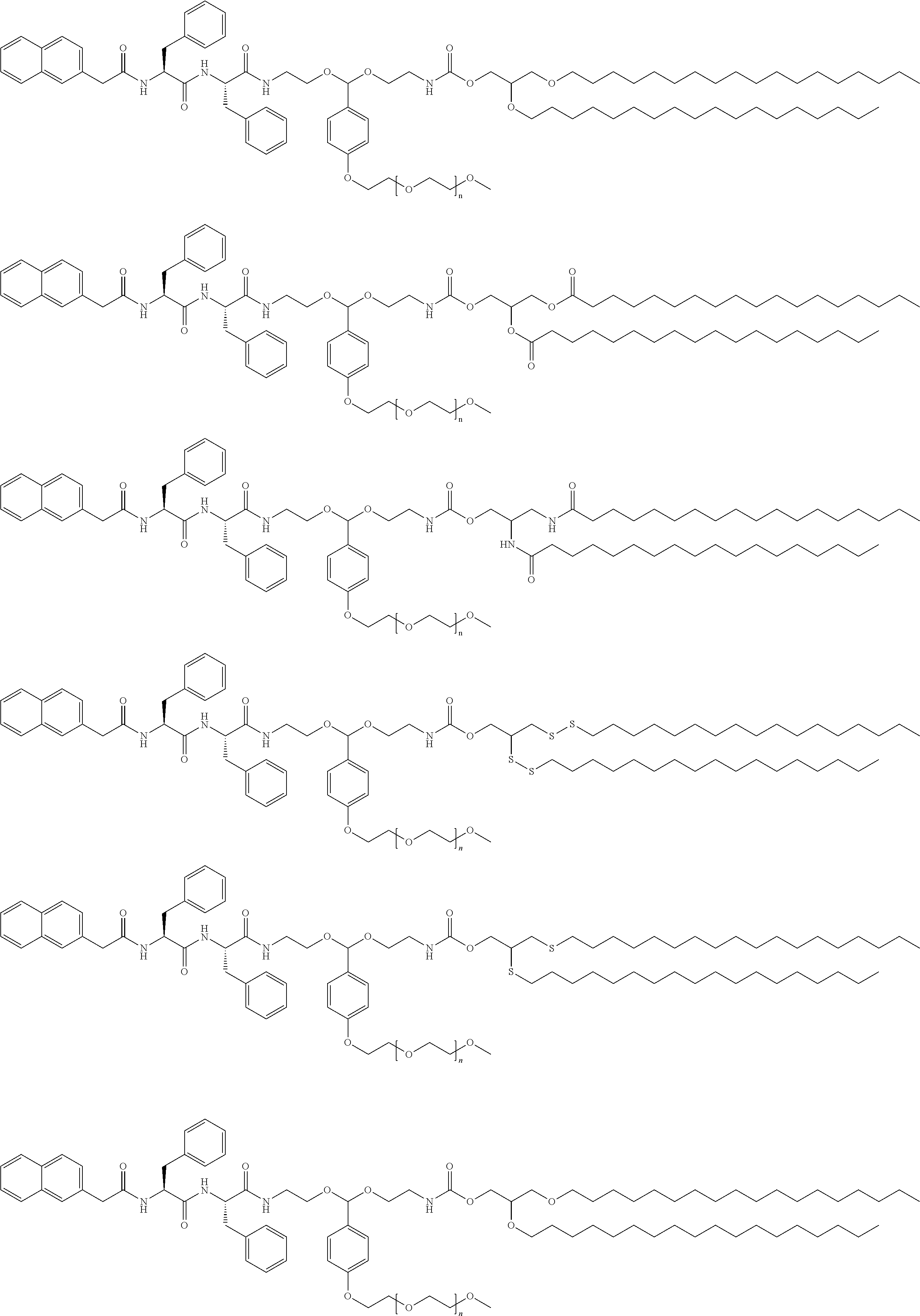

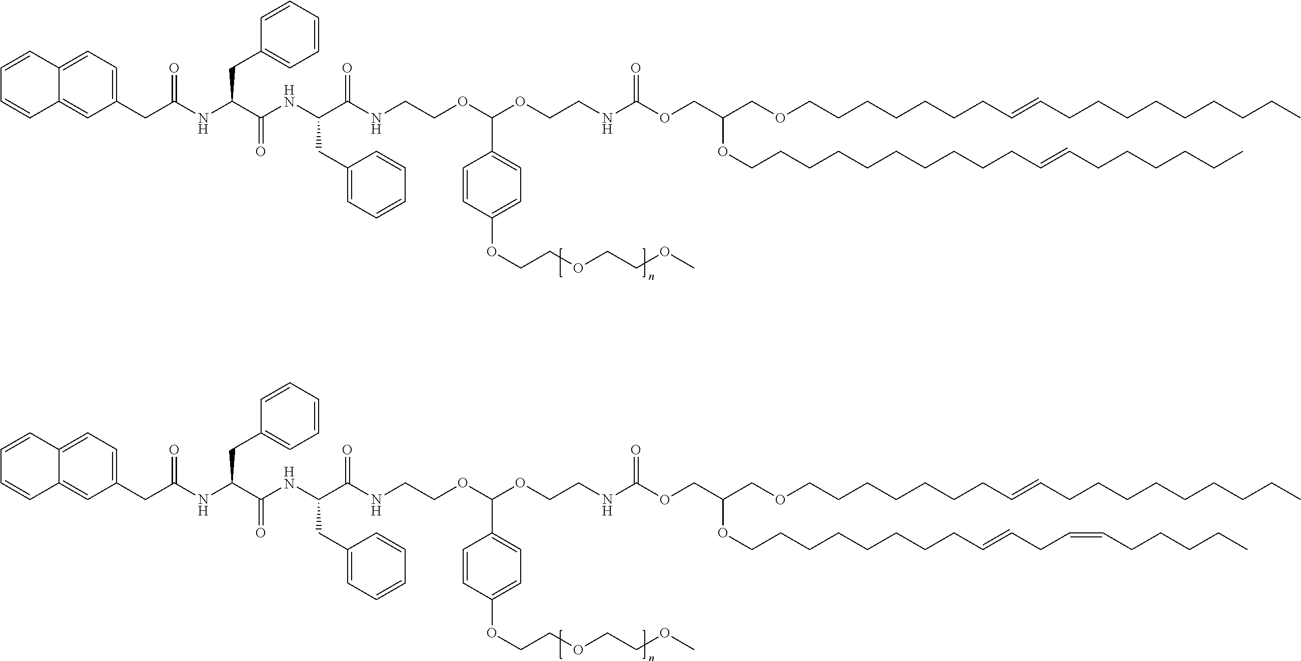

14. A composition of any of claims 1-8, wherein R2 comprises three alkyl chains:

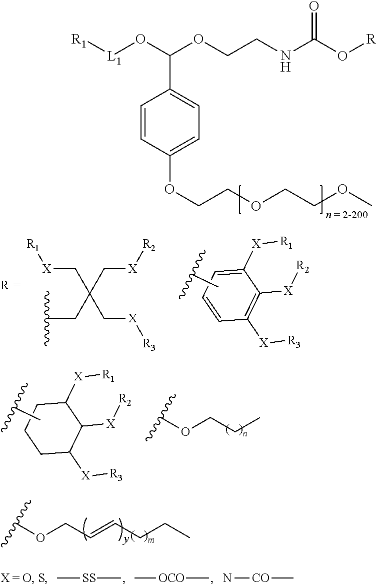

n=4-16; m=1-13; y=1-3, wherein the olefin(s) can be Z/E, and in any position(s) of the chain, such as:

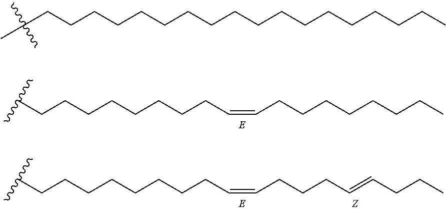 [SMILES: *CCCCCCCCCCCCCCCCCC ; *CCCCCCCC/C=C\CCCCCCCC ; *CCCCCCCC/C=C\CCC/C=C/CCC]

15. A composition of any of claims 1-8, wherein R2 comprises three alkyl chains, of structure:

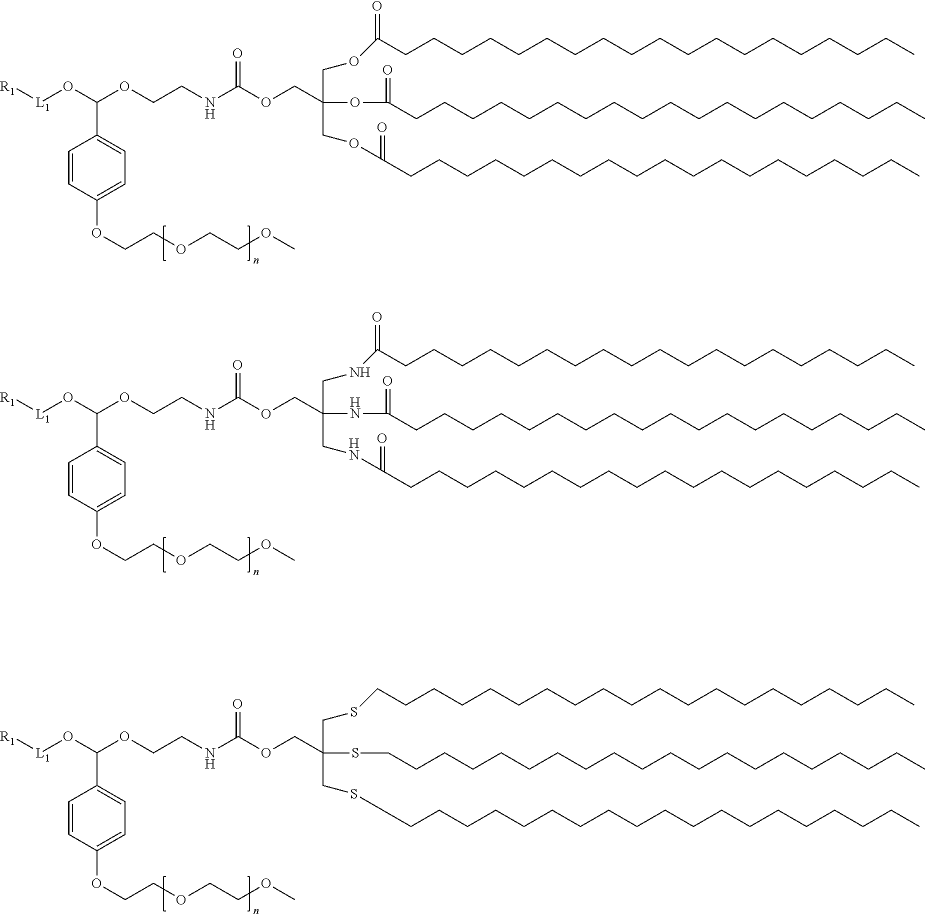

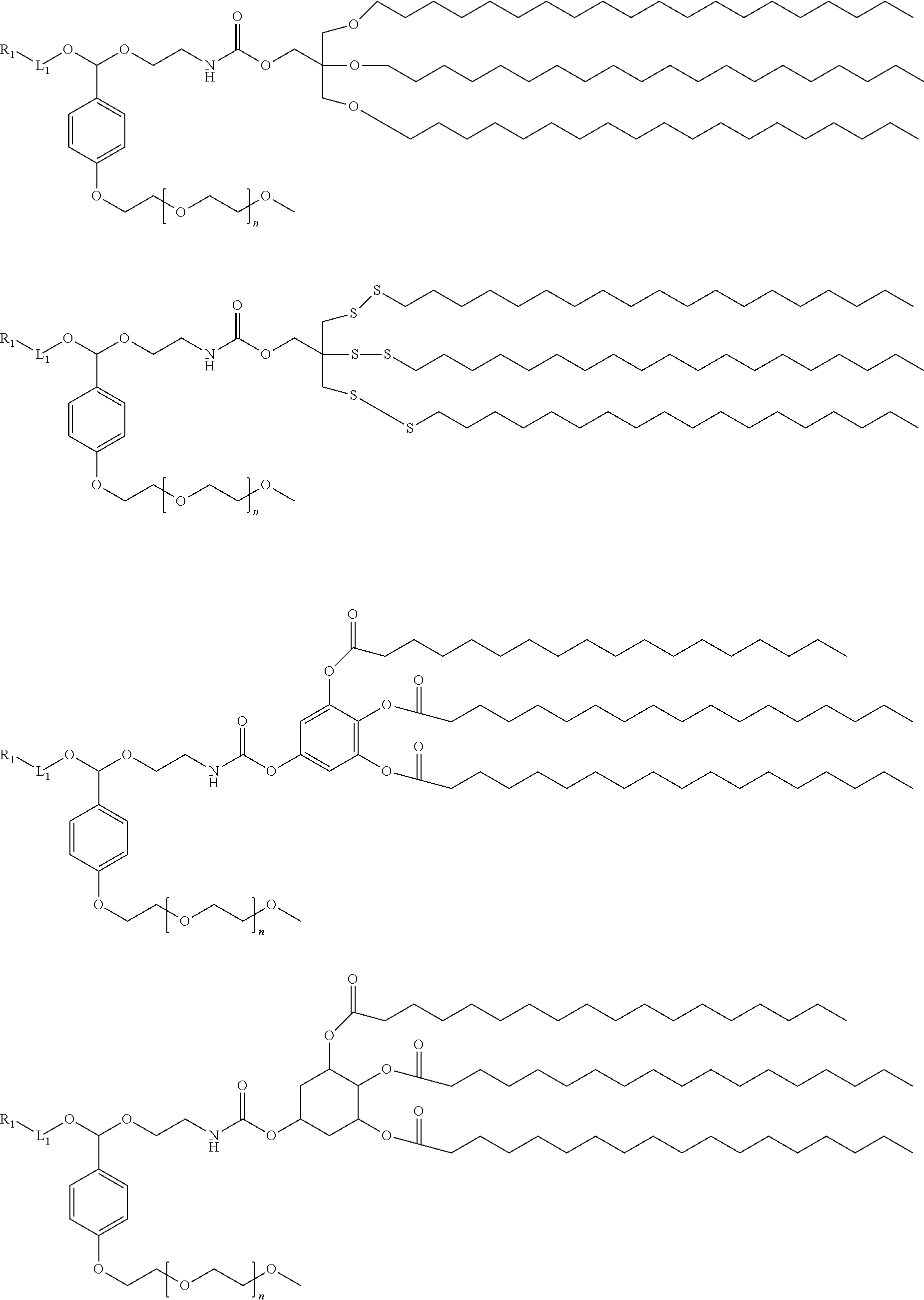

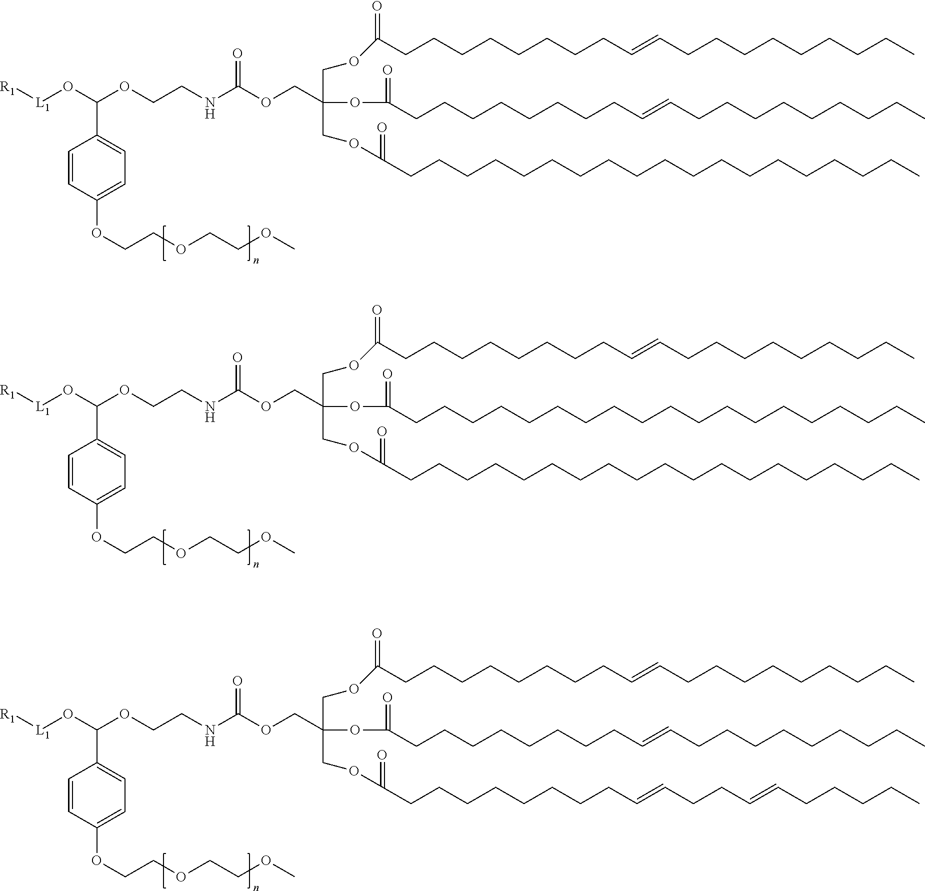

such as in:

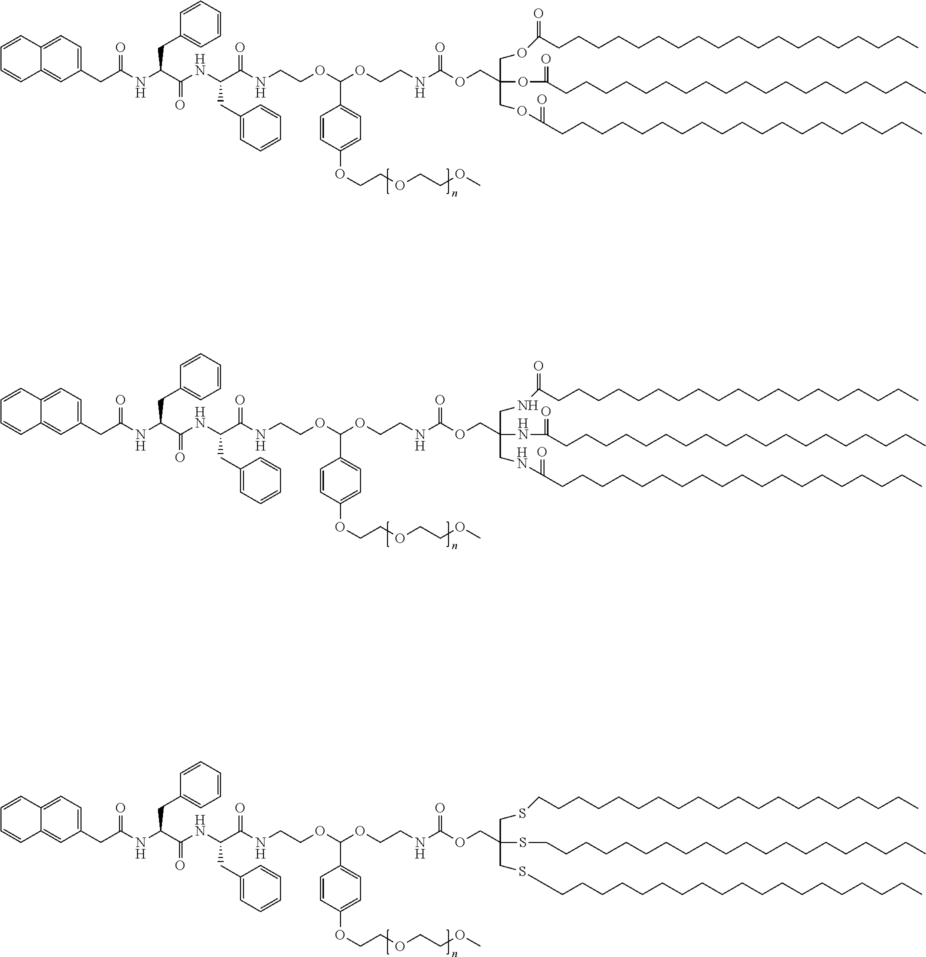

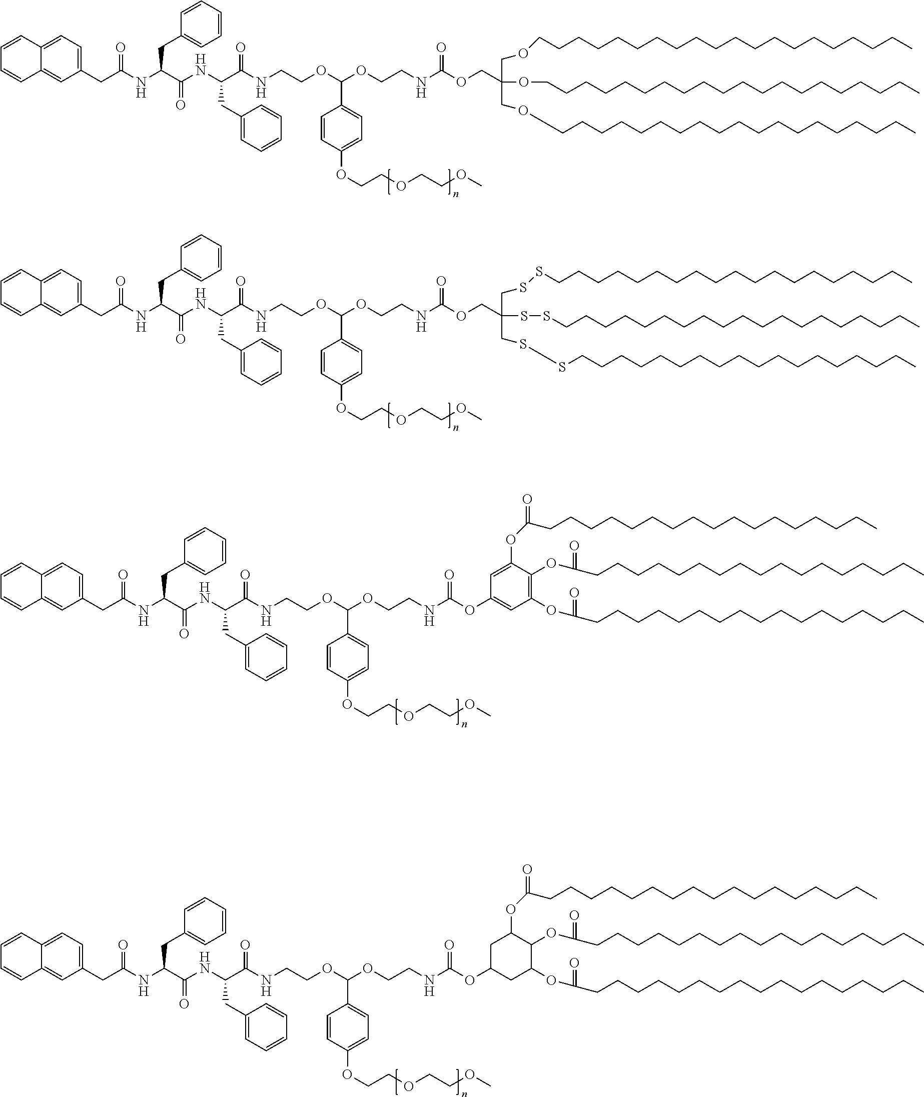

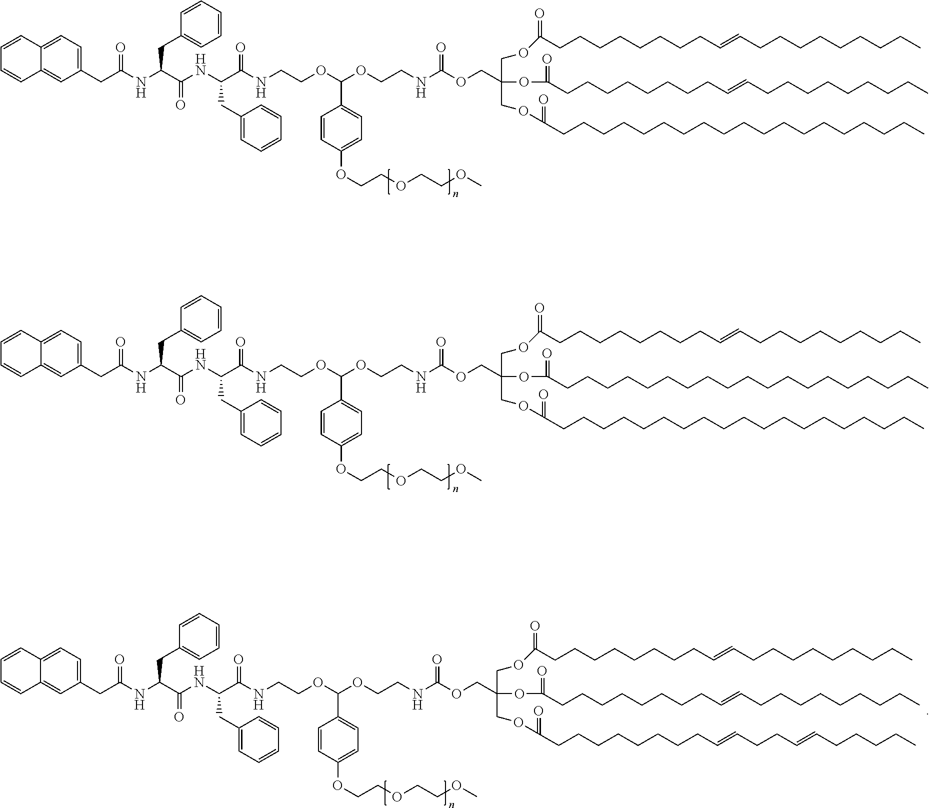

16. A composition of any of claims 1-8, wherein:

L1, L2 and L3 comprise linkers independently selected from optionally hetero-, optionally substituted linear C1-C12 alkyl.
17. A composition of any of claims 1-8, wherein:

L1, L2 and L3 comprise linkers independently selected from:

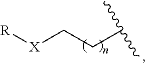

n=1-5; X=N, O, S; or

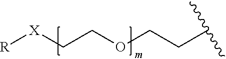

m=0-5, X=N, O, S.
18. A composition of any of claims 1-8, wherein:

L1 and L2 are —CH2CH2NH—R1 and —CH2CH2NHCO—R2, and L3 is a bond.
19. A composition of any of claims 1-8, wherein:

R3 comprises PEG of 500-5000 Da (e.g. 45-134 oxyethylene units).
20. A composition of any of claims 1-8, wherein:

L3 comprises a triazole.
21. A composition of any of claims 1-8, wherein:

L3 comprises a triazole click reaction product between an acetal alkyne and PEG-N3, e.g.:

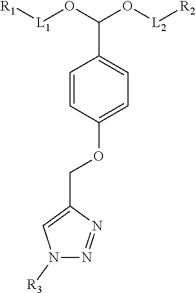

linked by amide bond with the peptide on the amino side.
22. A composition of any of claims 1-8, wherein L3 provides a direct link with R3 (e.g. a PEG), of structure:

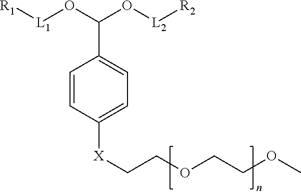

### X=N, O, S, —OCO—, —SCO—, SS, —NCO—

n=2-200

such as in:

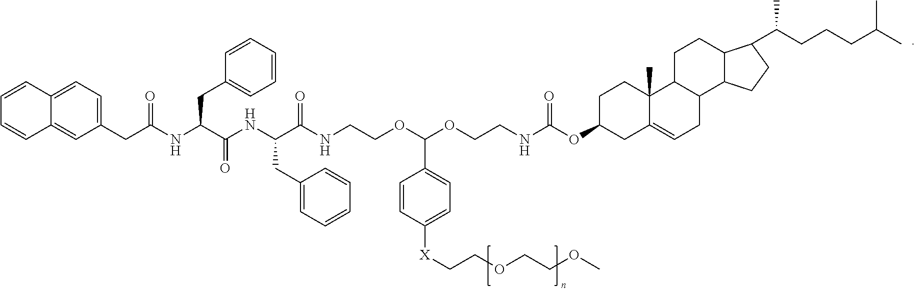

23. A composition of any of claims 1-8, wherein L3 provides a direct link with R3 (e.g. a PEG), of structure:

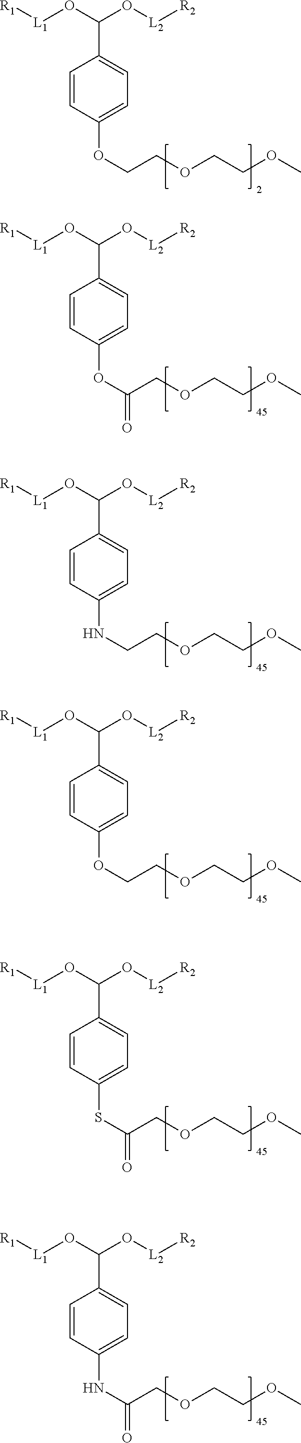

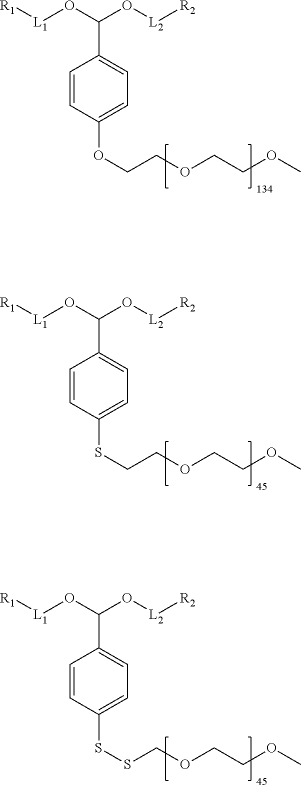

such as in:

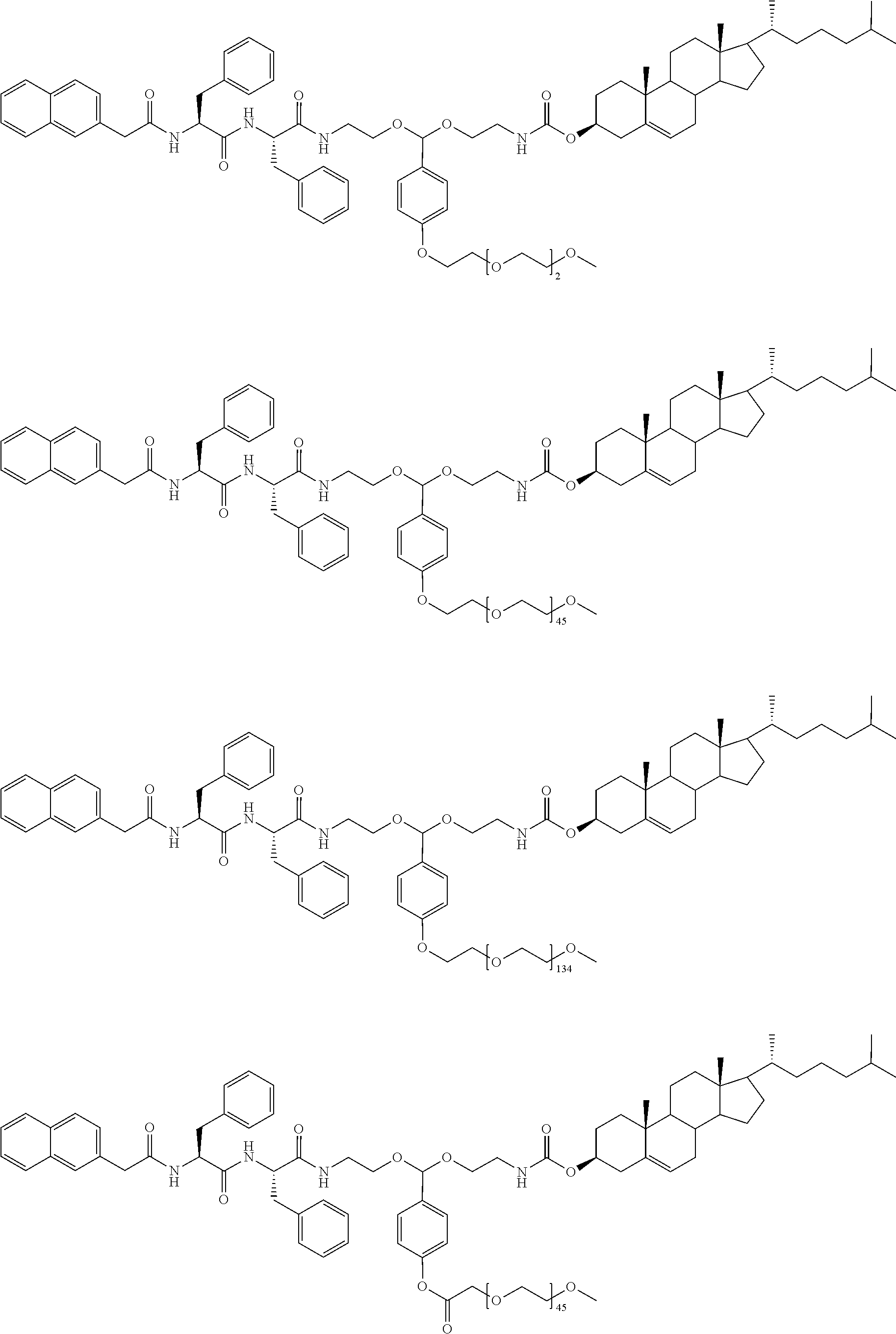

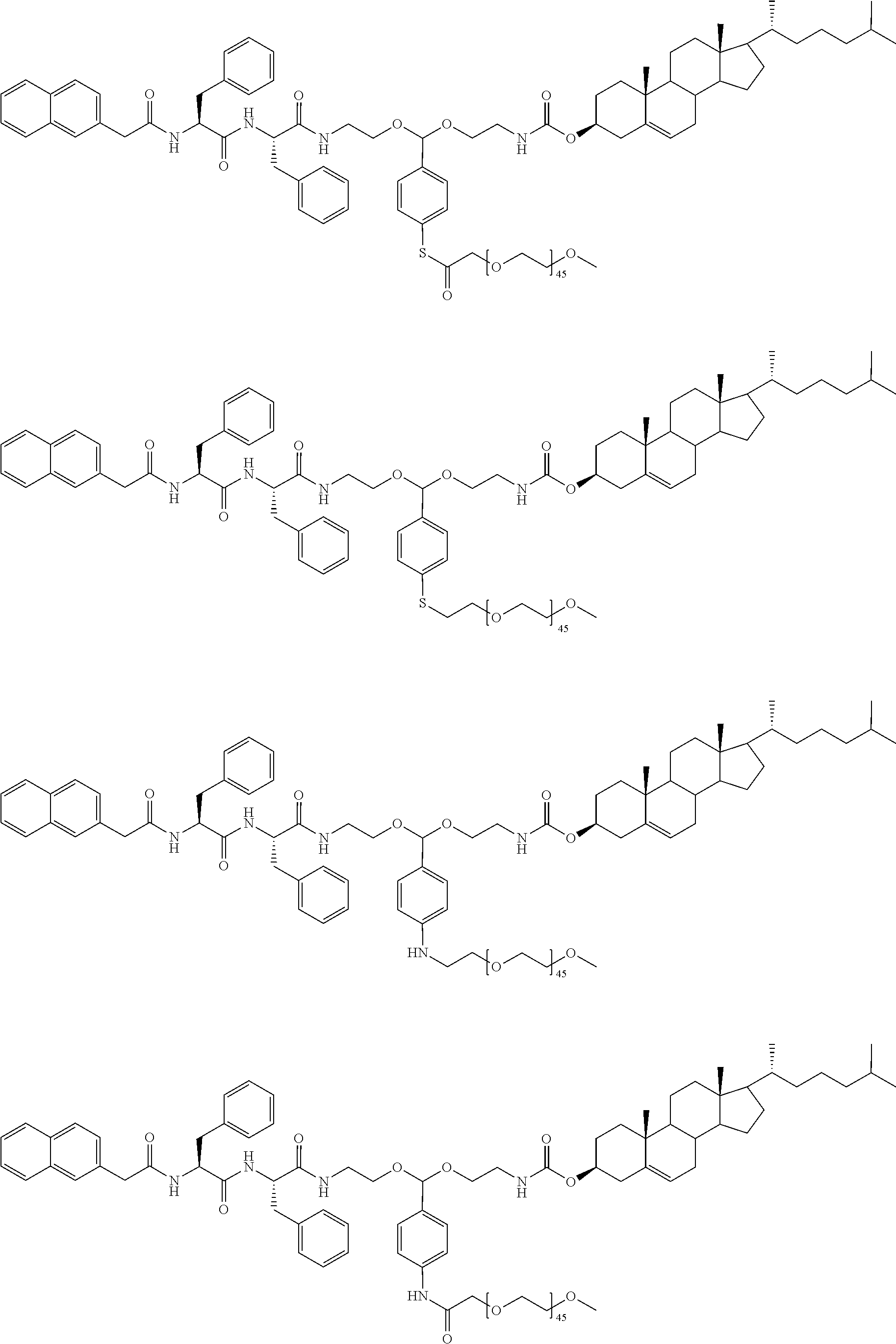

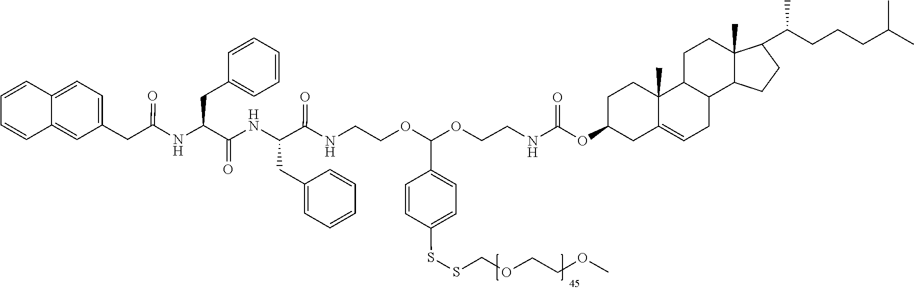

24. A composition of any of claims 1-8. wherein L3 provides a link with R3 (e.g. PEG) by click reaction, of structure:

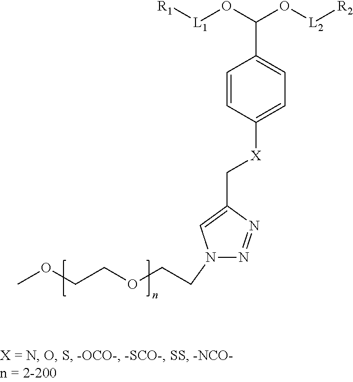

such as in:

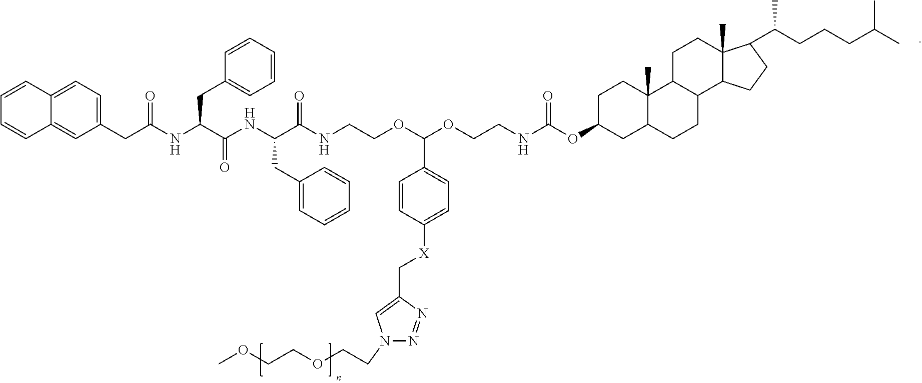

25. A composition of any of claims 1-8. wherein L3 provides a link with R3 (e.g. PEG) by click reaction, of structure:

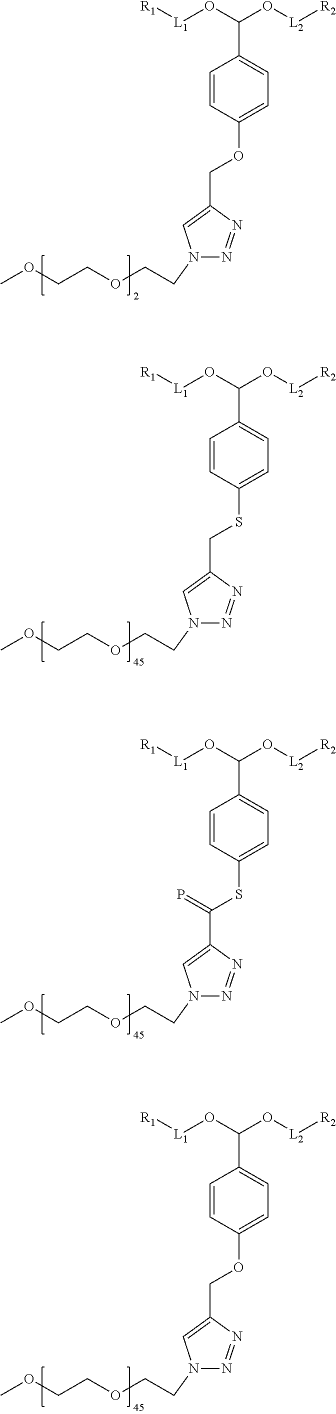

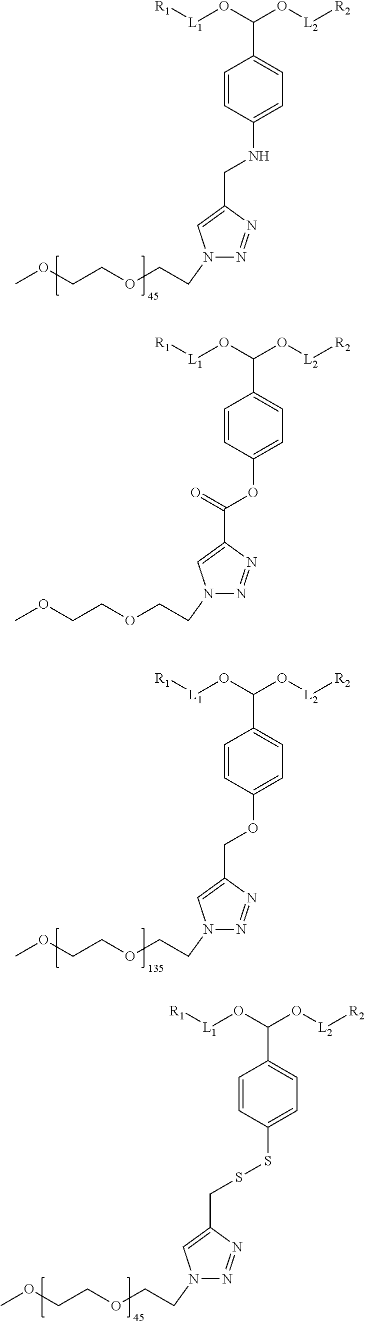

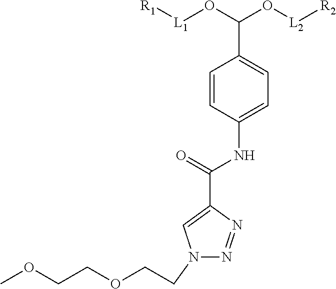

such as in:

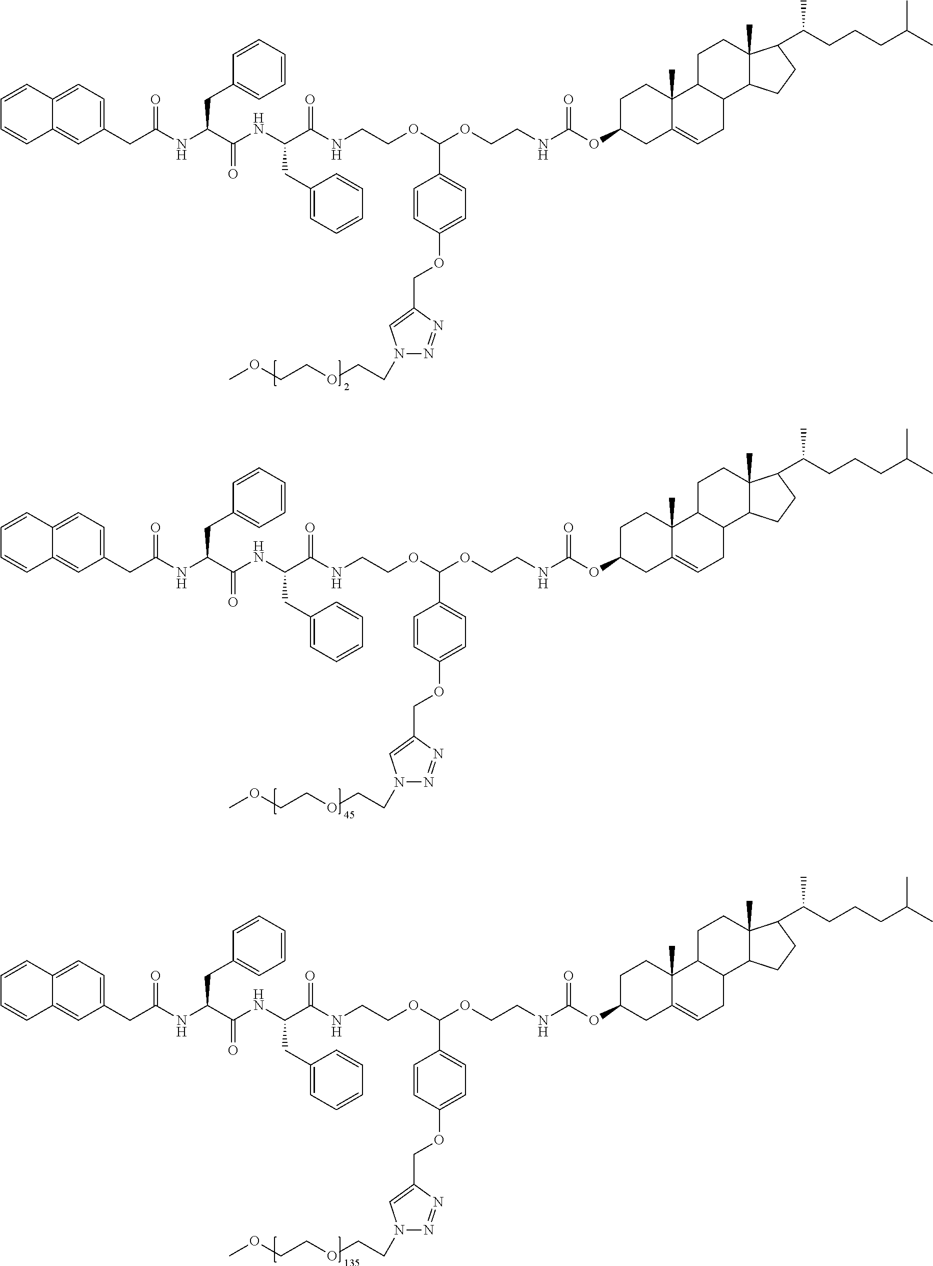

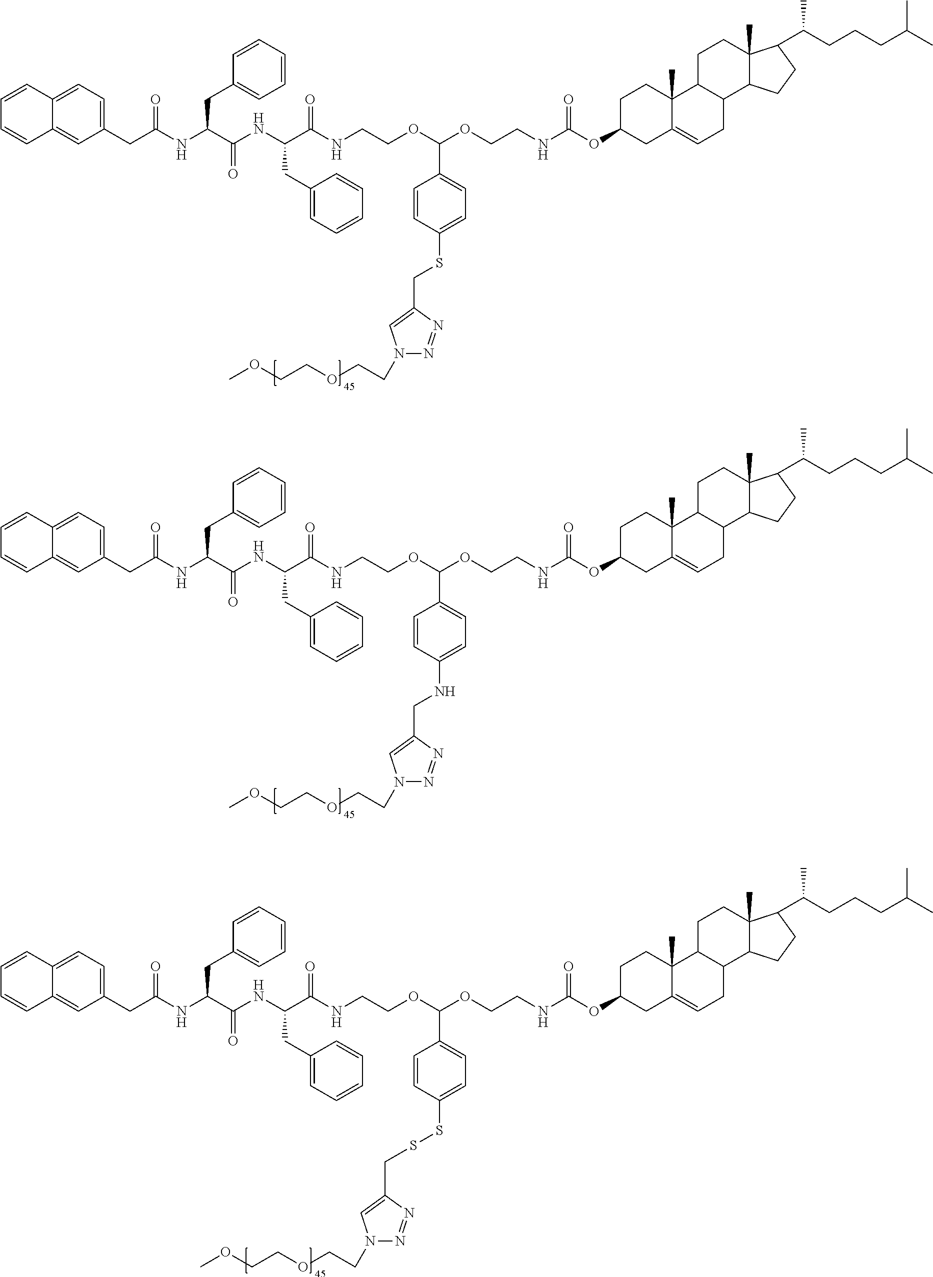

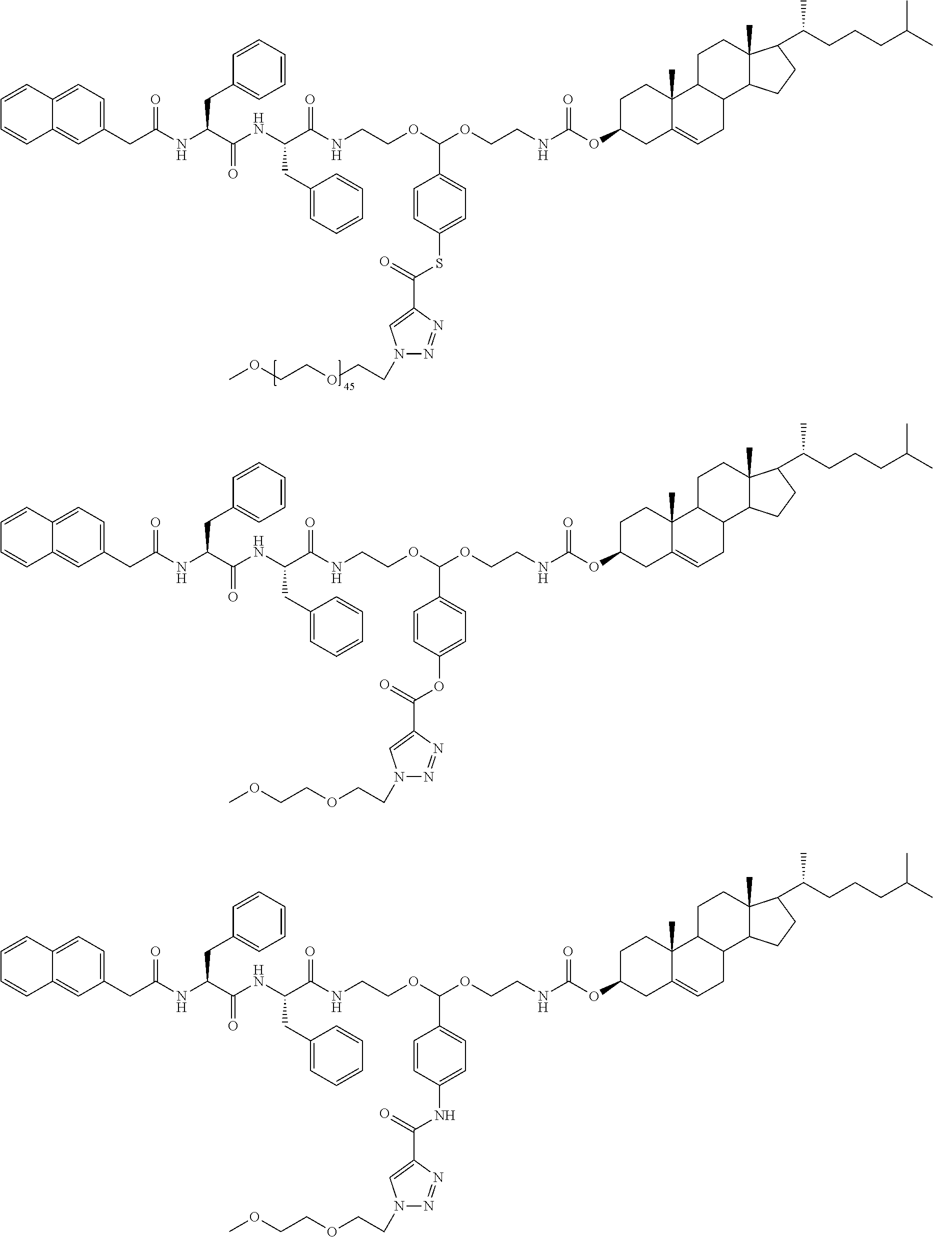

26. A composition of any of claims 1-8. comprising the structure of Pep-Chol-PEG-1k/2k:

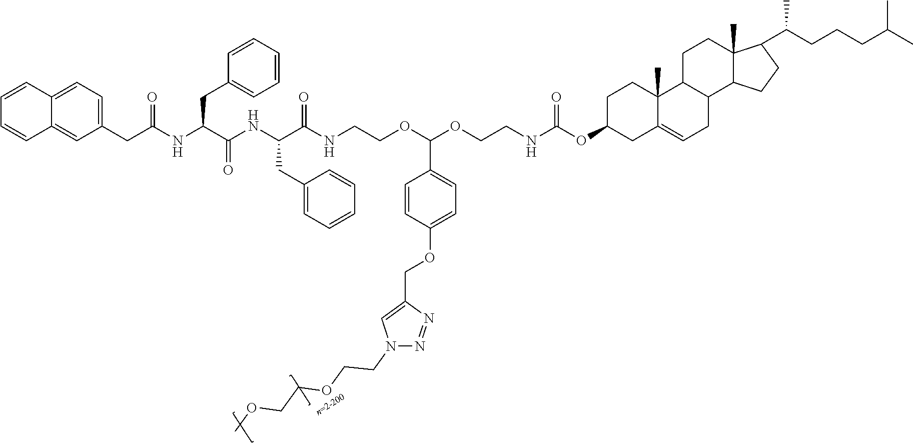

27. A method of making a compound of any of claims 1-8, comprising a triazole click reaction, wherein L3 comprises a triazole click reaction product between an acetal alkyne and PEG-N3, e.g.:

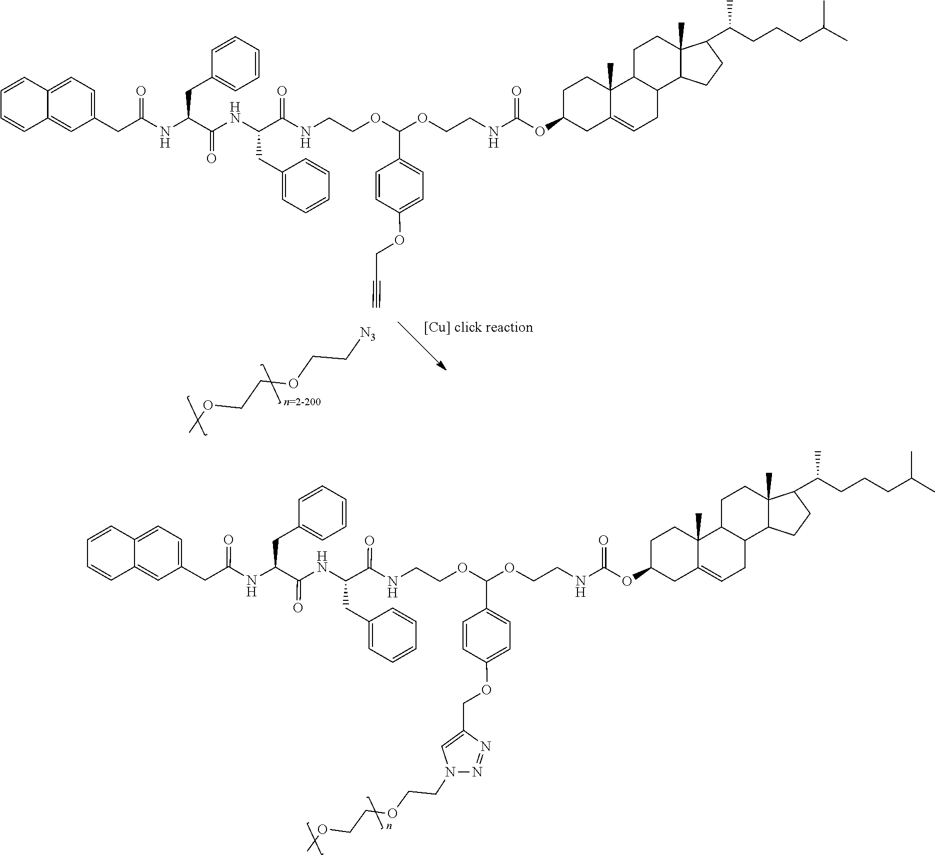

28. A lipid nanoparticle (LNP) composition comprising a compound of any of claims 1-8, configured, for example, to deliver mRNA, plasma DNA, siRNA, for example, for vaccines, wherein for some of the mRNA, like Cas 9 mRNA may be combined with guide RNA, for cell editing and treating disease.
29. A composition of any of claims 1-8, formulated into solid lipid nanoparticles (SLNs) further comprising a nucleic acid, such as an RNA or DNA, encoding a therapeutic protein, vaccine antigen, or gene editing enzyme(s).
30. A method of using a compound of any of claims 1-8, comprising delivering the compounds in a lipid nanoparticle (LNP) composition comprising a compound herein, to transfect a tissue or organ, such as muscle, lung, spleen, liver and blood.
31. A method of using a compound of any of claims 1-8, comprising delivering the compounds in a lipid nanoparticle (LNP) composition comprising a compound herein configured as a vaccine or therapeutic, and preferably detecting a resultant intended, targeted effect, and preferably with enhanced effect, e.g. mRNA transfection efficiency attributable to use of the compound.
32. A method of using a compound of any of claims 1-8, wherein the acid cleavable linker is deployed to cage the self-assembling peptide, and releasing in an endosome (which can help/enhance mRNA transfection efficiency).
33. A method of using a compound of any of claims 1-8, wherein the compound is cleaved substantially according the mechanism:

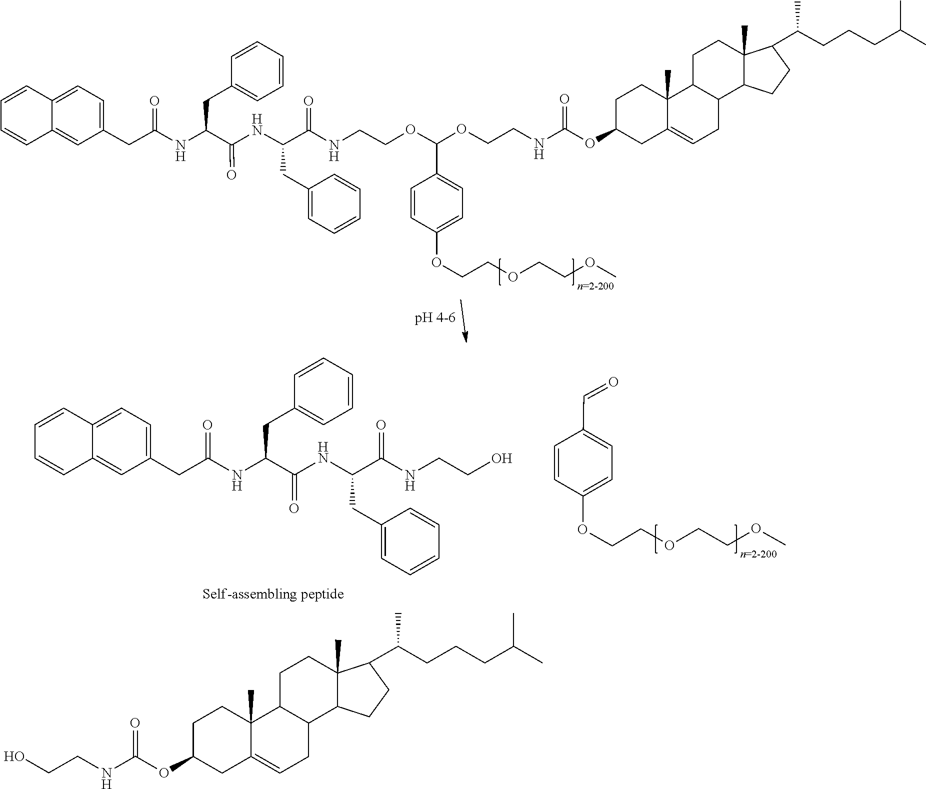

34. A method of using a compound of any of claims 1-8, wherein a resultant change in morphology is determined, such as by a method of morphology determination substantially:
a) Reaction: 3.15 mg of compound 1 is dissolved in 100 μL of DMSO (10 mM). Then 10 μL of the solution is added to 190 μL of PBS buffer (pH5) to a final concentration of 500 μM. This reaction mixture is incubated at 37° C. for 2 hours; and
b) TEM sample preparing: Firstly, a carbon Formvar-coated copper grid was placed on a drop of reaction sample. After adsorption for about 8 min, the grid was removed from the solution and then placed on a drop of uranyl acetate solution (2% w/v) for negative staining. The samples were subjected to TEM characterization immediately after preparation.
35. A method of making a compound of any of claims 1-8, comprising solid phase peptide synthesis.
36. A solid-phase lipid synthesis method comprising synthesis of cationic, ionizable lipids via solid phase peptide synthesis.
37. The method of claim 36 comprising integration in an automated robotic system (ARS) of: (i) the solid phase lipid synthesis, (ii) initial cell screening, and (iii) animal organ or cell targeting.
38. A compound, composition or method as described herein.

The invention encompasses all combinations of the particular embodiments recited herein, as if each combination had been laboriously recited.

### BRIEF DESCRIPTION OF THE DRAWINGS

FIG. 1. NMR spectrum of the 1-1K

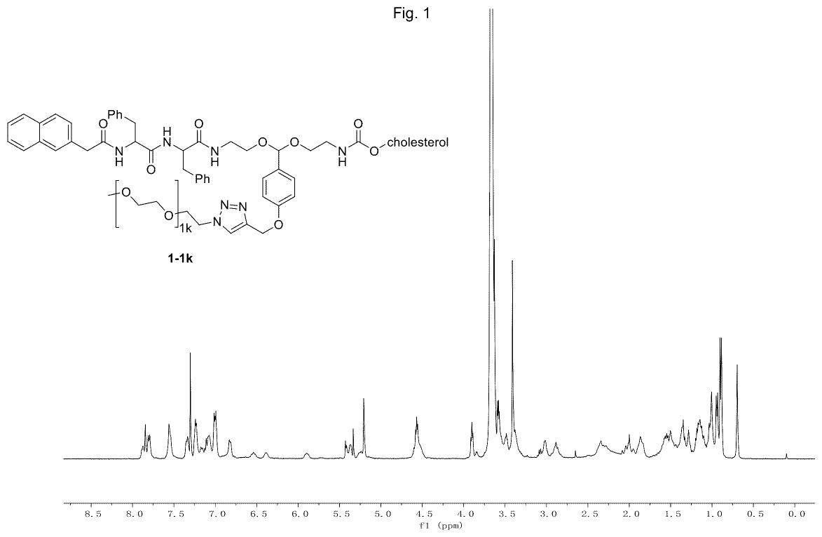

FIG. 2. NMR spectrum of the 1-2K.

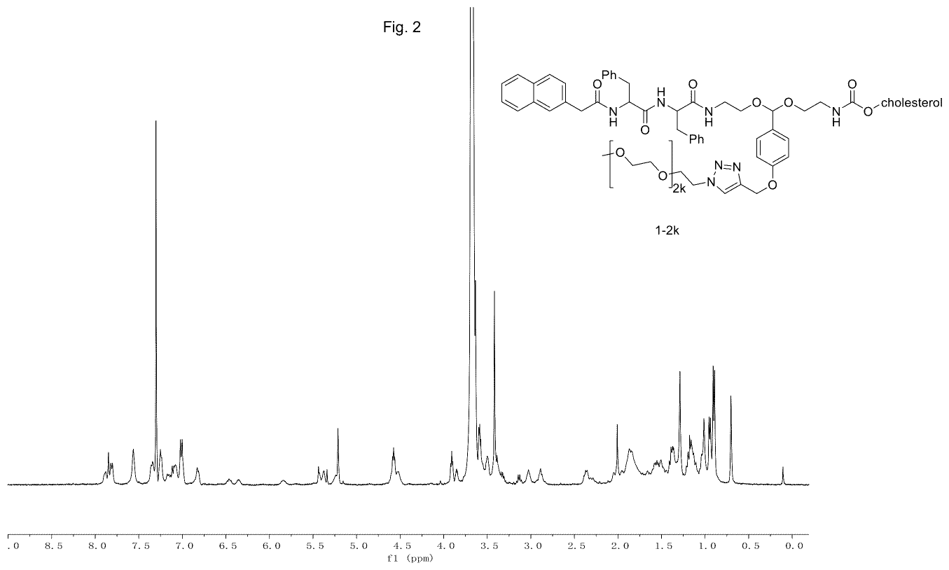

FIG. 3. HEK cells with 300 ng. EGFP mRNA

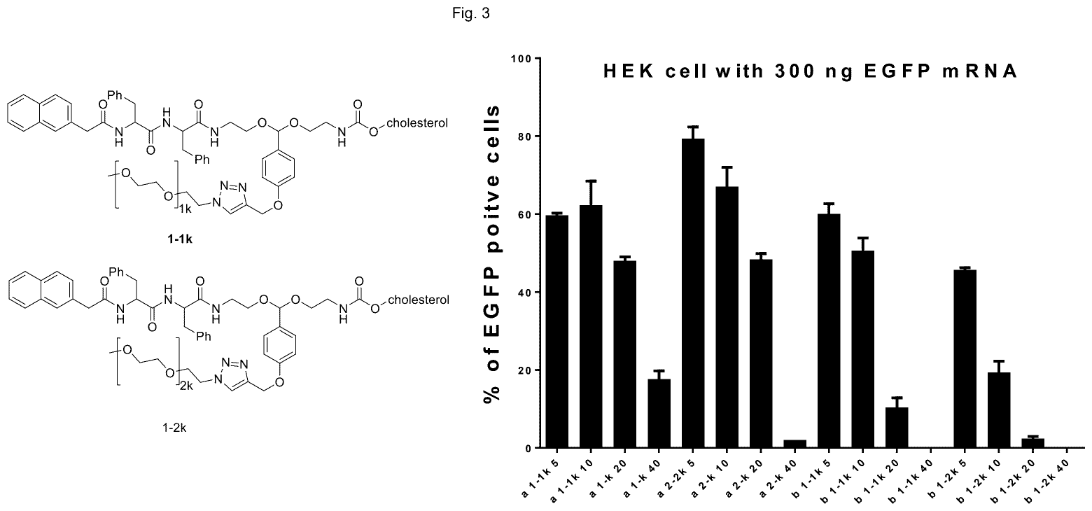

FIG. 4. In vivo results: IV Formula A 10 ug mRNA

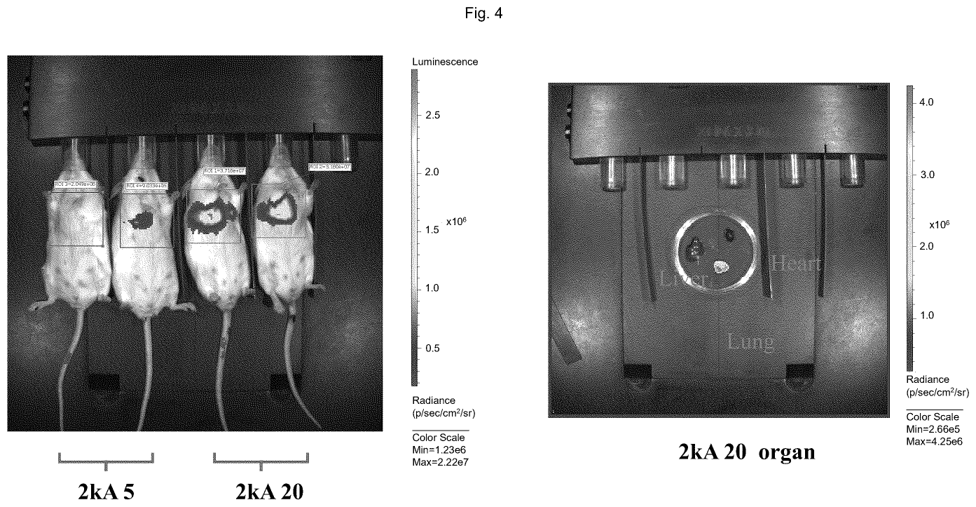

FIG. 5. In vivo results: IV Formula B 10 ug mRNA

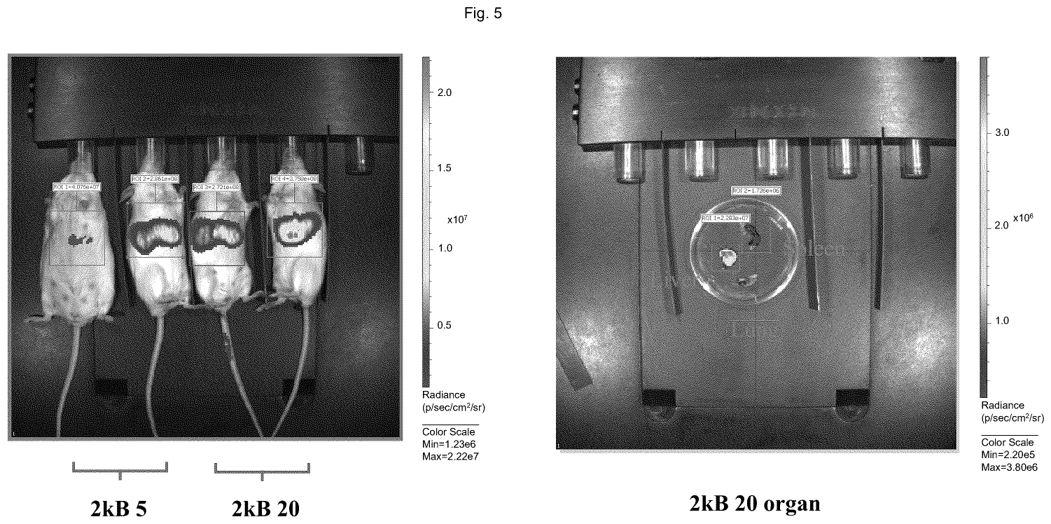

FIG. 6. Left panel; HEK 293T cells with 300 ng eGFP mRNA, 10,000 cell per well, serum free medium, 24 H; Right panel: Jurkat cell with 300 ng eGFP mRNA, 20,000 cell per well, full serum medium, 24 H.

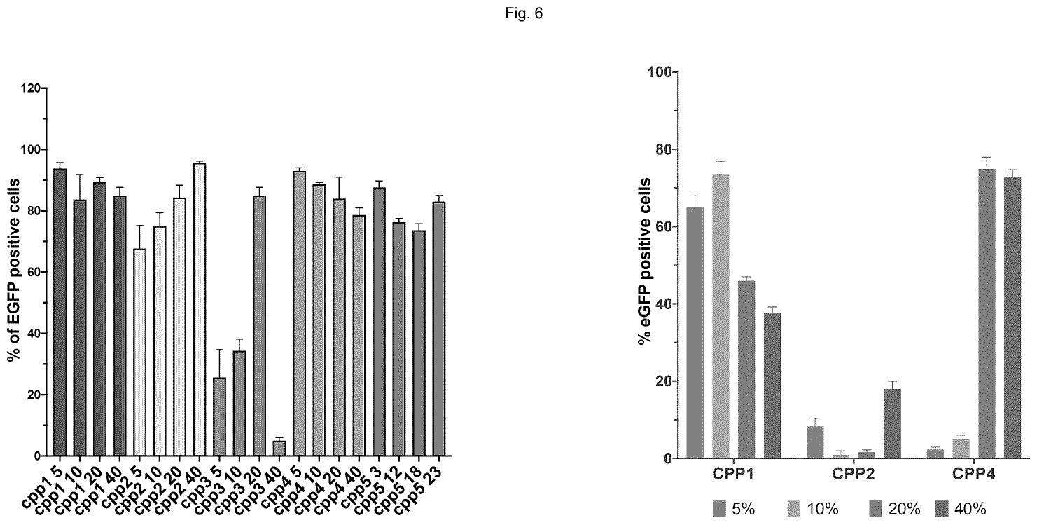

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

Lipid nanoparticles with representative acid degradable PEG-lipids (500 DA, 1 kD, and 2 kD PEG) transfect mRNA in HELA and HEK cells efficiently, and transfect muscle tissue efficiently, and transfect systemically in mice after intravenous injection, transfect cells in the blood, transfect mRNA into brain tissue after an intracranial injection, transfect lung tissue with specificity; and transfect spleen tissue with specificity after an intravenous injection.

Exemplary experimental Protocols. LNPs made with representative compounds deliver luciferase mRNA and CRE mRNA to the lungs with specificity. LNPs containing various mole ratios of compounds are screened in mice for their ability to deliver luciferase mRNA, after an intravenous injection (10 ug mRNA per mouse). These results are further validated via separate CRE mRNA delivery experiments using Ai9 mice. Ai9 mice are given three consecutive injections of CRE mRNA (0.5 mg/kg per dose, 2 days apart) at 10 ug mRNA per dose, and two days after the last injection the mice are sacrificed, and the lung tissue is analyzed for fluorescence and compared against saline treated Ai9 mice. Ai9 mice treated with LNPs containing representative compounds had numerous red cells in their lung histology sections.

These results indicate that LNPs made with these compounds can deliver ASOs and CFTR mRNA to lung tissue.

### Examples: New Acid Degradable Lipids Based on Self Assembling Peptides

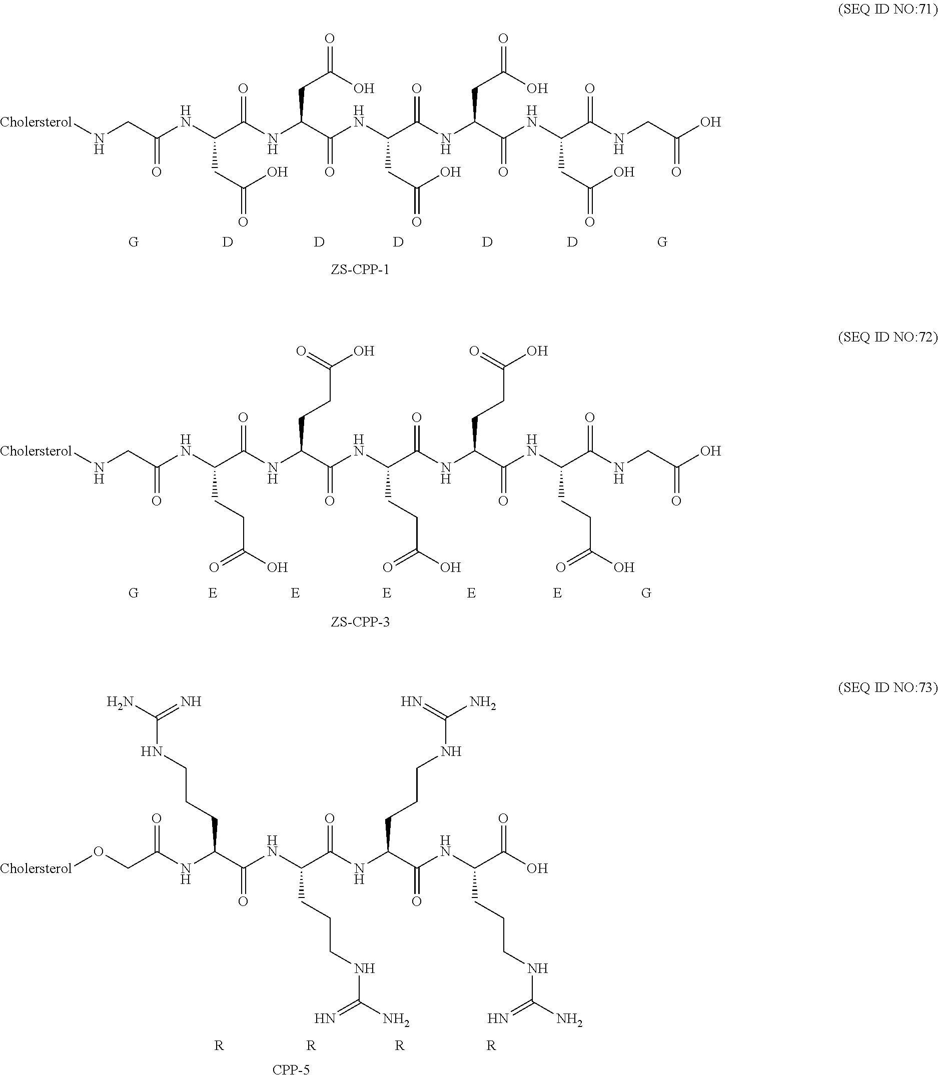

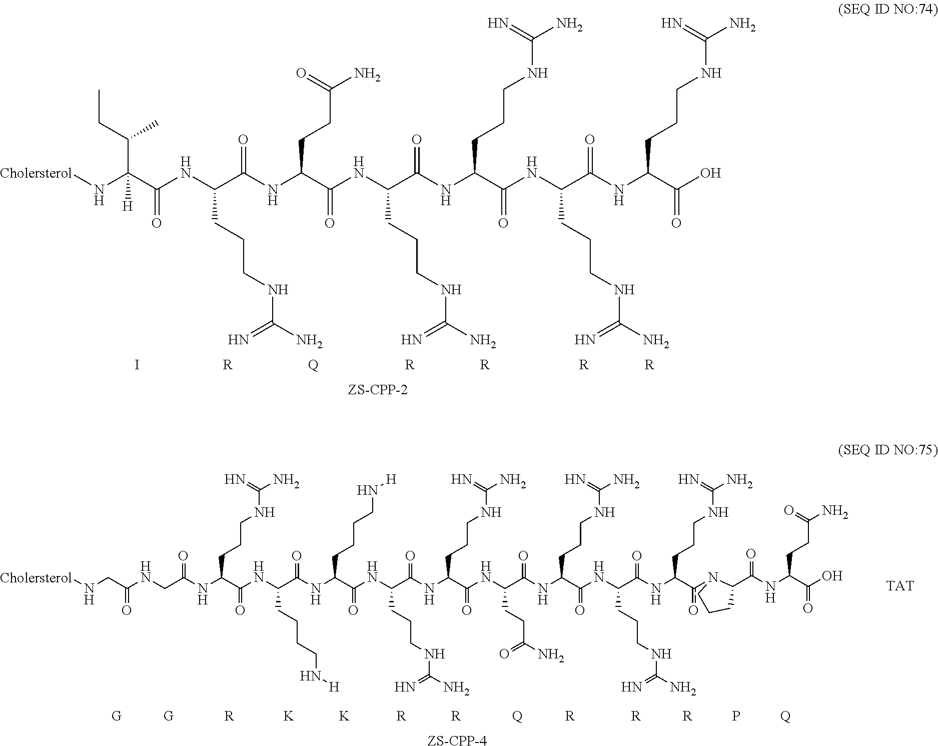

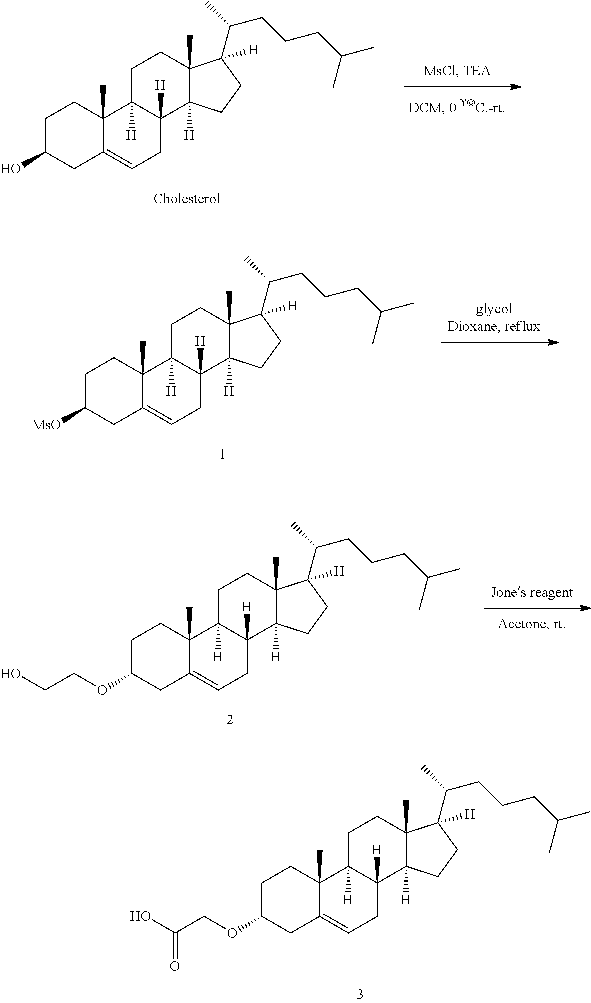

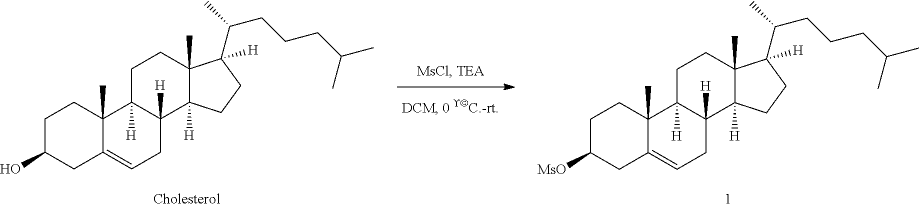

Cholesterol (20 g, 51.73 mmol, 1.0 equiv) was added to a round bottom flask with 400 mL at room temperature, followed by adding triethylamine (14.35 mL, 103.5 mmol, 2 equiv). Then methane sulfonyl chloride (4.8 mL, 62.1 mmol, 1.2 equiv) was added dropwise in ice bath under vigorously stirring. Monitor the reaction by TLC, generally the reaction will complete 30 min. When completed, the reaction was washed by 0.5 M HCl (100 mL×3), saturated aqueous NaHCO₃ (50 mL×3) and NaCl brine (100 mL×2), dried the organic phase by MgSO₄, then removal of the solvent under reduced pressure. The concentrated residue was recrystallized by diethyl ether. The crude product (yield quantitative, off-white powder) was used in the next step without further purification.

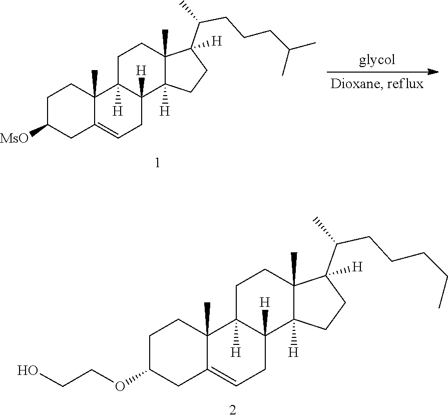

To a solution of cholesteryl mesylate (51.73 mmol) in dry 1,4-dioxane (350 mL), glycol (85 mL, 1.552 mol, 30 equiv) was added and refluxed for about 2 hours until complete disappearance of the starting material (monitored by TLC). After concentration of the reaction mixture, the residue was dissolved in 200 mL of DCM. Solution was washed with saturated aqueous NaHCO₃ (100 mL×3), water (100 mL×3) and NaCl brine (100 mL×2). The organic phase was dried with sodium MgSO₄, solvent was removed under reduced pressure. The concentrated residue was recrystallized by DCM and hexane. The crude product (17.4 g, 78%, off-white powder) was used in the next step without further purification.

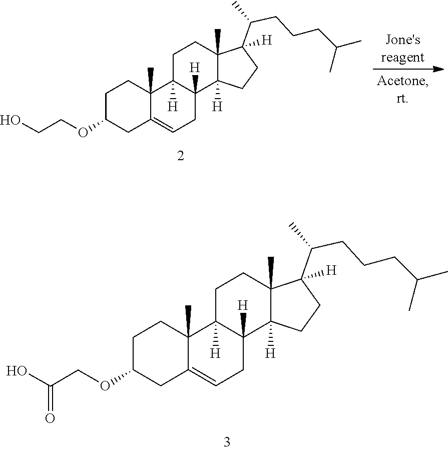

To a solution of cholesteryl glycol (17 g, 39.47 mmol, 1.0 equiv) in 500 mL of acetone, 2M Jones reagent (32 mL, 63.15 mmol, 1.6 equiv) was added while stirring. The mixture was monitored by TLC. 1 hour later, additional 2M Jones reagent (8 mL, 15.78 mmol, 0.4 equiv) was added and the reaction was kept stirring at room temperature over 3 hours. Then green precipitate was filtered off and solvent evaporated to dryness. Residue was dissolved in DCM, washed with saturated sodium NaCl brine (100 mL×2). and dried with sodium MgSO₄. Then it was filtered through Celite and evaporated to dryness. The partial of the product can be purified by recrystallization (dichloride methane and hexane, 8.5 g, 37%), product in the filtrate can be purified by the column chromatography on silica gel (DCM/MeOH gradient 0-15%). Yields 6.4 g (28%)

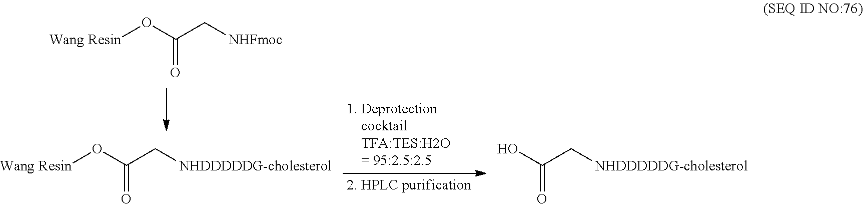

Typical synthetic procedure (for CCP1): Wang Resin (150 mg, 0.0585 mmol, 0.39 mmol/g) was added to the reactor. Concentration of synthons:

| Amino acids | Cholesterol acid | HBTU | Piperidine |
|---|---|---|---|
| 0.4M | 0.25M | 0.4M | 0.8M |

Solvent is DMF

The peptide synthesis was performed on an Aapptech Eclipse solid phase peptide synthesizer, using standard FMOC synthesis procedures, following the manufacturers recommended procedure. The cholesterol acid was added as the last amino acid at 0.25 Molar concentration, on the solid phase.

| | | |
|---|---|---|
| Components | MW | Volume/μL (mole %) |

Cpp1 formula

| | | | | | |
|---|---|---|---|---|---|
| ZS-CPP-1 | 1134.2 | 0.25 (5%) | 0.54 (10%) | 1.2 (20%) | 3.2 (40%) |
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp2 formula

| | | | | | |
|---|---|---|---|---|---|
| ZS-CPP-2 | 1466.93 | 0.33 (5%) | 0.69 (10%) | 1.56 (20%) | 4.15 (40%) |
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp3 formula

| | | | | | |
|---|---|---|---|---|---|
| ZS-CPP-3 | 1204.38 | 0.27 (5%) | 0.57 (10%) | 1.28 (20%) | 3.4 (40%) |
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp4 formula

| | | | | | |
|---|---|---|---|---|---|
| ZS-CPP-4 | 2105.67 | 0.47(5%) | 1.0 (10%) | 2.23 (20%) | 5.95(40%) |
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Cpp5 formula

| | | | | | |
|---|---|---|---|---|---|
| ZS-CPP-5 | 1068 | 0.14 (3%) | 0.62 (12%) | 1.0 (18%) | 1.35 (23%) |
| PEG2K-DMG | 2500 | 0.5 | 0.5 | 0.5 | 0.5 |
| DOPE | 744.3 | 3 | 3 | 3 | 3 |
| D-Lin | 642.09 | 4 | 4 | 4 | 4 |
| Chol | 386.64 | 2.5 | 2.5 | 2.5 | 2.5 |

Concentration of PEG2K-DMG, DOPE,D-Lin, choleterol, DOTAP are 10 mg/mL in ethanol. The concentration of All choleterol-peptides are 40 mg/mL in DMSO

For LNP preparation a stock solution (10 mg mL⁻¹) of D-Lin, DOPE, Cholesterol, DMG-PEG and was made by dissolving each lipid separately in ethanol and all cholesterol-peptide lipids were 40 mg mL⁻¹ in DMSO stored at −30° C. Just before the LNP formation, lipids were taken out and kept on ice and vortexed whenever necessary. The cholesterol solution was slightly warmed up to dissolve the crystals which form during the cold storage. Then D-Lin, DOPE, Cholesterol, DMG PEG and cholesterol-peptide lipids were mixed in variety molar ratios listed in table 1. RNAs 1 μg μL⁻¹ (Luciferase-mRNA, eGFP mRNA) were mixed with LNP and PBS (pH 7.2) in a 1:1:2 ratio (vol/vol). The resulting LNPs were gently vortexed and incubated at room temperature for 15 minutes for immediate use or stored at 4° C.

LNP in vivo: BALB/c mice with age of 16-24 weeks were i.v. (retro-orbital) injected with various luciferase mRNA LNP formulations. LNPs were formed as described in table 1 and mice were i.v. (retro-orbital) injected with a dose of 10 μg Luc mRNA per mouse. After 5 h, mice were injected with d-Luciferin (150 mg kg⁻¹, intraperitoneal) and imaged using an IVIS Lumina system (Perkin Elmer)

LNP in vitro: HEK 293T cells were seeded in 96 well plate at 10⁵ cells mL⁻¹ and cultured with DMEM medium (10% FBS and 1% P.S) under 5% CO₂ at 37° C. for 18-24 h before transfection. LNPs were formed as described in table 1 and 300 ng eGFP mRNA was added to each well with 100 μL Opti serum free medium. GFP-positive cells were quantified by FACS (Attune) analysis 24 after treatment.

Jurkat cell were diluted to 2×10⁵ cells mL⁻¹ and cultured with RPMI1640 medium (10% FBS and 1% P.S) under 5% CO₂ at 37° C. LNPs were formed as described in table 1 and 300 ng eGFP mRNA was added to each well with 100 μL full serum medium with 20,000 cells per well. GFP-positive cells were quantified by FACS (Attune) analysis 24 after treatment.

### Synthesis of Cationic and Ionizable Lipids Via Solid Phase Peptide Synthesis

Conventional chemical de novo synthesis cationic and ionizable is slow, expensive and inefficient. We demonstrate that cationic lipids and ionizable lipids can be synthesized using standard solid phase peptide synthesis methodology, e.g. Fields CG et al., Peptide Research, 1 Mar. 1991, 4(2):95-101, PMID: 1815783, HBTU activation for automated Fmoc solid-phase peptide synthesis; Behrendt et al, J. Pept. Sci. 2016;22:4-27, Advances in Fmoc solid-phase peptide synthesis. We also disclose integration in an automated robotic system (ARS) of: (i) the solid phase lipid synthesis, (ii) initial cell screening, and (iii) animal organ or cell targeting.

This methodology has several advantages over conventional organic synthesis of cationic lipids and expands the types of cationic/ionizable lipids and lipid libraries that can be synthesized. It does not require a trained organic chemist and can be performed automatically and robotically and lowers the cost, time and chemical space that can be accessed, typically by a factor of at least 10 or 100.

### Formulations

| 1KA | | | | 1KB | | | |
|---|---|---|---|---|---|---|---|
| Lipid1-5% | | | | Lipid1-5% | | | |
| Standard | MW | Volume | % | Standard | MW | Volume | % |
| 1-1K | 2139 | 1 | 5.094113 | 1K | 2139 | 0.96 | 5.036032 |
| mPEG- | 2500 | 0.5 | 1.089631 | mPEG- | 2500 | 0.5 | 1.122091 |
| DMG | | | | DMG | | | |
| DOTAP | 663.1 | 3 | 24.64856 | DOPE | 744.3 | 3 | 22.61368 |
| Chol | 386.64 | 2.5 | 35.22756 | Chol | 386.64 | 2.5 | 36.27699 |
| D-Lin | 642.09 | 4 | 33.94013 | D-Lin | 642.09 | 4 | 34.9512 |
| Lipid1-10% | | | | Lipid1-10% | | | |
| Standard | MW | Volume | % | Standard | MW | Volume | % |
| 1-1K | 2139 | 2.08 | 10.04321 | 1K | 2139 | 2.01 | 9.993722 |
| mPEG- | 2500 | 0.5 | 1.032809 | mPEG- | 2500 | 0.5 | 1.063511 |
| DMG | | | | DMG | | | |
| DOTAP | 663.1 | 3 | 23.3632 | DOPE | 744.3 | 3 | 21.43311 |
| D-Lin | 642.09 | 4 | 32.17024 | D-Lin | 642.09 | 4 | 33.12654 |
| Chol | 386.64 | 2.5 | 33.39054 | Chol | 386.64 | 2.5 | 34.38312 |
| Lipid1-20% | | | | Lipid1-20% | | | |
| Standard | MW | Volume | % | Standard | MW | Volume | % |
| 1-1K | 2139 | 4.68 | 20.07679 | 1K | 2139 | 4.54 | 20.05068 |
| mPEG- | 2500 | 0.5 | 0.917612 | mPEG- | 2500 | 0.5 | 0.944678 |
| DMG | | | | DMG | | | |
| DOTAP | 663.1 | 3 | 20.75733 | DOPE | 744.3 | 3 | 19.03826 |
| D-Lin | 642.09 | 4 | 28.58204 | D-Lin | 642.09 | 4 | 29.42511 |
| Chol | 386.64 | 2.5 | 29.66623 | Chol | 386.64 | 2.5 | 30.54128 |
| Lipid1-30% | | | | Lipid1-30% | | | |
| Standard | MW | Volume | % | Standard | MW | Volume | % |
| 1-1K | 2139 | 7.99 | 30.01446 | 1K | 2139 | 7.78 | 30.05877 |
| mPEG- | 2500 | 0.5 | 0.803516 | mPEG- | 2500 | 0.5 | 0.826423 |
| DMG | | | | DMG | | | |
| DOTAP | 663.1 | 3 | 18.17635 | DOPE | 744.3 | 3 | 16.65504 |
| D-Lin | 642.09 | 4 | 25.02814 | D-Lin | 642.09 | 4 | 25.74166 |
| Chol | 386.64 | 2.5 | 25.97753 | Chol | 386.64 | 2.5 | 26.71811 |
| Lipid1-40% | | | | Lipid1-40% | | | |
| Standard | MW | Volume | % | Standard | MW | Volume | % |
| 1-1K | 2139 | 12.43 | 40.01867 | 1K | 2139 | 12.1 | 40.06274 |
| mPEG- | | | | mPEG- | | | |
| DMG | 2500 | 0.5 | 0.688656 | DMG | 2500 | 0.5 | 0.708217 |
| DOTAP | 663.1 | 3 | 15.5781 | DOPE | 744.3 | 3 | 14.2728 |
| D-Lin | 642.09 | 4 | 21.45045 | D-Lin | 642.09 | 4 | 22.05973 |
| Chol | 386.64 | 2.5 | 22.26412 | Chol | 386.64 | 2.5 | 22.89651 |
| 1-2K | | | | | | | |
| 2KA 2KB | | | | | | | |
| Lipid1-5% | | | | Lipid1-5% | | | |
| Standard | MW | Volume | % | Standard | MW | Volume | % |
| 1-2K | 3139 | 1.44 | 5.003403 | 2K | 3139 | 1.4 | 5.006123 |
| mPEG- | 2500 | 0.5 | 1.090672 | mPEG- | 2500 | 0.5 | 1.122444 |
| DMG | | | | DMG | | | |
| DOTAP | 663.1 | 3 | 24.67212 | DOPE | 744.3 | 3 | 22.6208 |
| Chol | 386.64 | 2.5 | 35.26123 | Chol | 386.64 | 2.5 | 36.28842 |
| D-Lin | 642.09 | 4 | 33.97257 | D-Lin | 642.09 | 4 | 34.96221 |
| Lipid1-10% | | | | Lipid1-10% | | | |
| Standard | MW | Volume | % | Standard | MW | Volume | % |
| 1-2K | 3139 | 3.04 | 10.00645 | 2K | 3139 | 2.97 | 10.05561 |
| mPEG- | 2500 | 0.5 | 1.033231 | mPEG- | 2500 | 0.5 | 1.06278 |
| DMG | | | | DMG | | | |
| DOTAP | 663.1 | 3 | 23.37275 | DOPE | 744.3 | 3 | 21.41837 |
| D-Lin | 642.09 | 4 | 32.18339 | D-Lin | 642.09 | 4 | 33.10376 |
| Chol | 386.64 | 2.5 | 33.40418 | Chol | 386.64 | 2.5 | 34.35947 |
| Lipid1-20% | | | | Lipid1-20% | | | |
| Standard | MW | Volume | % | Standard | MW | Volume | % |
| 1-2K | 3139 | 6.86 | 20.05824 | 2K | 3139 | 6.66 | 20.04469 |
| mPEG- | 2500 | 0.5 | 0.917825 | mPEG- | 2500 | 0.5 | 0.944749 |
| DMG | | | | DMG | | | |
| DOTAP | 663.1 | 3 | 20.76214 | DOPE | 744.3 | 3 | 19.03968 |
| D-Lin | 642.09 | 4 | 28.58868 | D-Lin | 642.09 | 4 | 29.42731 |
| Chol | 386.64 | 2.5 | 29.67312 | Chol | 386.64 | 2.5 | 30.54356 |
| 1-2K | 3139 | 11.72 | 30.0048 | 2K | 3139 | 11.4 | 30.02706 |
| mPEG- | 2500 | 0.5 | 0.803627 | mPEG- | 2500 | 0.5 | 0.826798 |
| DMG | | | | DMG | | | |
| DOTAP | 663.1 | 3 | 18.17886 | DOPE | 744.3 | 3 | 16.66259 |
| D-Lin | 642.09 | 4 | 25.0316 | D-Lin | 642.09 | 4 | 25.75333 |
| Chol | 386.64 | 2.5 | 25.98111 | Chol | 386.64 | 2.5 | 26.71286 |
| Lipid1-40% | | | | Lipid1-40% | | | |
| Standard | MW | Volume | % | Standard | MW | Volume | % |
| 1-2K | 3139 | 18.26 | 40.0435 | 2K | 3139 | 17.76 | 40.067 |
| mPEG- | 2500 | 0.5 | 0.688371 | mPEG- | 2500 | 0.5 | 0.708166 |
| DMG | | | | DMG | | | |
| DOTAP | 663.1 | 3 | 15.57165 | DOPE | 744.3 | 3 | 14.27179 |
| D-Lin | 642.09 | 4 | 21.44157 | D-Lin | 642.09 | 4 | 22.05816 |
| Chol | 386.64 | 2.5 | 22.25491 | Chol | 386.64 | 2.5 | 22.89488 |

### Synthesis of Model compound S3

Compound S3 and the tripeptide were made based on the reported references,

J. Am. Chem. Soc. 2017, 139, 6, 2306-2317; Tetrahedron 2007, 63, 7349-7357

### Synthesis of Model compound S4

To a solution of S3 (2.0 g, 7.48 mmol, 1.0 equiv) in 50 mL dichloride methane, was added the solution of cholesteryl chloroformate (2.38 g, 5.56 mmol, 0.7 equiv, dissolved in 40 mL DCM) dropwise in an ice bath. After end of the adding, the reaction mixture was allowed to warm to room temperature and keep stirring at same temperature for 2 hours. Then the reaction mixture was concentrated under reduced pressure. The residue was purified by MeOH/DCM (0.1% TEA): (0-10%) on Biotage Selekt, to give S4 (2.1 g, 56%)

### Synthesis of Model compound S5

To a solution of S4 (100 mg, 0.148 mmol, 1.0 equiv), tripeptide (78 mg, 0.162 mmol, 1.1 equiv) and HATU (85 mg, 0.22 mmol, 1.5 equiv) in 5 mL DMF, was added DIPEA (52 μL, 0.295 mmol, 2 equiv). The reaction mixture was keep stirring at same temperature for 1 hours. Then the reaction mixture was concentrated under reduced pressure. The residue was purified by MeOH/DCM (0.1% TEA): (0-10%) on Biotage Selekt, to give S5 (120, mg, 66%)

### Synthesis of Model compound 1-1k and 1-2k

To a solution of S5 (50 mg, 0.044 mmol, 1.0 equiv), azide-PEG1K (88 mg, 0.044 mmol, 1.0 equiv) 3 mL DMF, was added sodium ascorbate (10.4 mg, 100 mg/ml in DDI water, 1.2 equivalent), and followed by adding CuSO₄ (9 mg, 100 mg/ml in DDI water, 0.6 equiv). The reaction mixture was keep stirring at same temperature overnight. Then the reaction mixture was concentrated under reduced pressure. The residue was purified by MeOH/DCM (0.1% TEA) by preparative TLC.

### Method for Morphology Determinations

Reaction: 3.15 mg of compound 1 is dissolved in 100 μL of DMSO (10 mM). Then 10 μL of the solution is added to 190 μL of PBS buffer (pH5) to a final concentration of 500 μM. This reaction mixture is incubated at 37° C. for 2 hours; and

b) TEM sample preparing: Firstly, a carbon Formvar-coated copper grid was placed on a drop of reaction sample. After adsorption for about 8 min, the grid was removed from the solution and then placed on a drop of uranyl acetate solution (2% w/v) for negative staining. The samples were subjected to TEM characterization immediately after preparation. For details, see, Li et al. Adv. Healthcare Mater. 2017, 6, 1601400.

### Delivery of mRNA in vivo

LNPs made with foregoing compounds deliver luciferase mRNA and CRE mRNA to the lungs with specificity. LNPs containing various mole ratios of compounds were screened in mice for their ability to deliver luciferase mRNA, after an intravenous injection (10 ug mRNA per mouse). These results were further validated via separate CRE mRNA delivery experiments using Ai9 mice. Ai9 mice are given three consecutive injections of CRE mRNA (0.5 mg/kg per dose, 2 days apart) at 10 ug mRNA per dose, and two days after the last injection the mice were sacrificed, and the tissues were analyzed for fluorescence and compared against saline treated mice.

Cpp5 3.3: High liver, low spleen, low lung

Cpp5 12: High liver; high spleen, low lung

Cpp5 18: High lung, low liver, low spleen

Cpp5 23: High Lung, low spleen, no liver

Cpp1 5: High liver, low spleen

Cpp1 40: High spleen, low liver

Cpp4 5: low liver

Cpp4 40: low liver

These results indicate that LNPs made with these compounds can deliver ASOs and CFTR mRNA in vivo with targetable tissue specificity.

### Synthetic Route

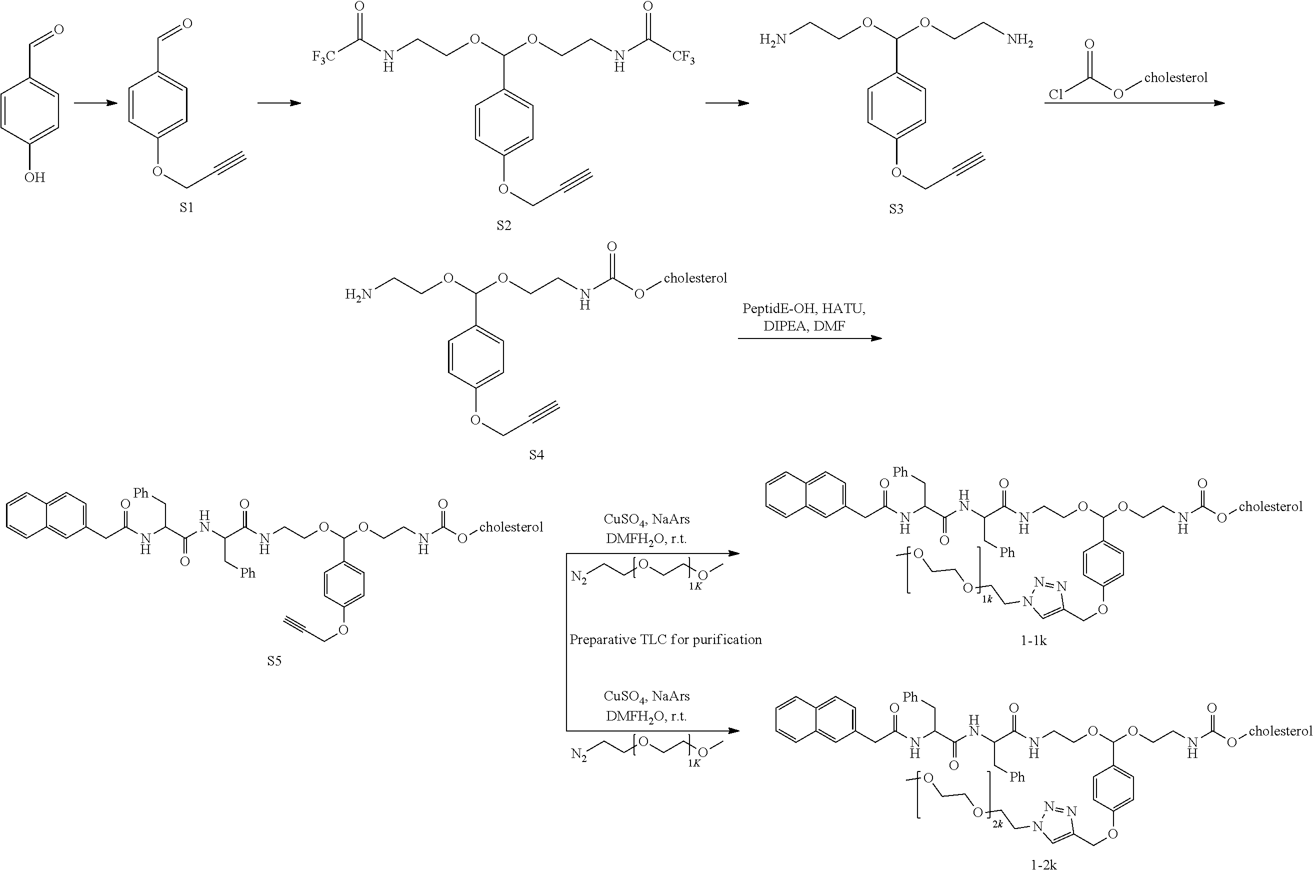

1. A composition comprising a compound comprising a benzaldehyde acetal acid-degradable amphiphilic lipid and a self-assembling peptide, of structure: 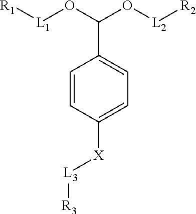
wherein:
R1 comprises a self-assembling peptide or precursor, which can self-assemble after the cleavage of the acid degradable acetal linker, wherein the self-assembled peptide preferably enhances endosome escape;
R2 comprises a hydrophobic group or lipid, such as a steroid (e.g. cholesterol) or one or more alkyl chains, such as in a single chain fatty acid or double or triple chain fatty acid ester, preferably that can strengthen the rigidity of a lipid nanoparticle, and also balance the amphiphilicity of the lipid;
R3 comprises a hydrophilic group, such as a PEG which can stabilize the lipid nanoparticle in serum, and extend the half-life of LNP-mRNA in a physiological environment, and when pairing with the hydrophobic group in the compound, the amphiphilicity of the lipid can be balanced, e.g. by changing the size of PEG;
X is O, S or N; and
L1, L2 and L3 are linkers selected from a bond, an optionally substituted heteroatom and an optionally substituted C1-18 hydrocarbyl or heterohydrocarbyl, providing acid degradable linkages.

2. A composition of claim 1, wherein incorporated in the compound the peptide is in the form of a non-self-assembling precursor that converts to a self-assembling peptide upon intracellular hydrolysis.

3. A composition of claim 1, wherein the peptide is configures so that upon hydrolysis the peptide can form intracellular nanofibrils that disrupt the dynamics of actin filament to cause either apoptosis or necroptosis of the cell, such as cancer cell, see, e.g. Li et al. Adv. Healthcare, Mater. 2017, 6, 1601400.

4. A composition of claim 1, wherein:
R1 comprises 1-10 amino acids, including any combination of D/L.

5. A composition of claim 1, wherein:
R1 comprises a self-assembling peptide selected from:
-NH-F(CO-)
-NH-FF(CO)
-NH-TF(CO)
-NH-YL(CO)
-NH-FFF(CO)
-NH-FFS(CO)
-NH-FFY(CO)
-NH-FFKY(CO-)
(SEQ ID NO: 01)
-NH-FFFY(CO-)
(SEQ ID NO: 02)
-NH-KYFF(CO-)
(SEQ ID NO: 03)
-NH-TFFY(CO-)
(SEQ ID NO: 04)
-NH-FFYY(CO-)
(SEQ ID NO: 05)
-NH-FYFY(CO-)
(SEQ ID NO: 06)
-NH-YFYF(CO-)
(SEQ ID NO: 07)
-NH-FFYE(CO-)
(SEQ ID NO: 08)
-NH-FFYR(CO-)
(SEQ ID NO: 09)
-NH-GFFY(CO-)
(SEQ ID NO: 10)
-NH-LLLY(CO-)
(SEQ ID NO: 11)
-NH-GFYY(CO-)
(SEQ ID NO: 12)
-NH-FFSY(CO-)
(SEQ ID NO: 13)
-NH-FFYS(CO-)
(SEQ ID NO: 14)
-NH-FFAGL(CO-)
(SEQ ID NO: 15)
-NH-FFAG(CO-)
(SEQ ID NO: 16)
-NH-FFYD(CO-)
(SEQ ID NO: 17)
-NH-FFEY(CO-)
(SEQ ID NO: 18)
-NH-LLLLY(CO-)
(SEQ ID NO: 19)
-NH-LLYLL(CO-)
(SEQ ID NO: 20)
-NH-FFGGY(CO-)
(SEQ ID NO: 21)
-NH-GFFYSV(CO-)
(SEQ ID NO: 22)
-NH-GFFYG(CO-)
(SEQ ID NO: 23)
-NH-FFGEY(CO-)
(SEQ ID NO: 24)
-NH-FFGDY(CO-)
(SEQ ID NO: 25)
-NH-FFEYI(CO-)
(SEQ ID NO: 26)
-NH-FFFFCG(CO-)
(SEQ ID NO: 27)
-NH-CEYFFG(CO-)
(SEQ ID NO: 28)
-NH-FFYGGAA(CO-)
(SEQ ID NO: 29)
-NH-GFFYGHY(CO-)
(SEQ ID NO: 30)
-NH-FENNQQNY(CO-)
SEQ ID NO: 31)
-NH-FFAGLDD(CO-)
(SEQ ID NO: 32)
-NH-FFYGGVV(CO-)
(SEQ ID NO: 33)
-NH-KRRASVAGK(CO-)
(SEQ ID NO: 34)
-NH-LYYYYL(CO-)
(SEQ ID NO: 35)
-NH-VVVD(CO-)
(SEQ ID NO: 36)
-NH-VVVK(CO-)
(SEQ ID NO: 37)
-NH-VVVVD(CO-)
(SEQ ID NO: 38)
-NH-VVVVDD(CO-)
(SEQ ID NO: 39)
-NH-VVVVWD(CO-)
(SEQ ID NO: 40)
-NH-VVVVWDD(CO-)
(SEQ ID NO: 41)
-NH-IIIIWDD(CO-)
(SEQ ID NO: 42)
-NH-LLLLWDD(CO-)
(SEQ ID NO: 43)
-NH-QQKFQFQFEQQ(CO-)
(SEQ ID NO: 44)
-NH-IIID(CO-)
(SEQ ID NO: 45)
-NH-IIIK(CO-)
(SEQ ID NO: 46)
-NH-LLLD(CO-)
(SEQ ID NO: 47)
-NH-LLLK(CO-)
(SEQ ID NO: 48)
-NH-GAVILEE(CO-)
(SEQ ID NO: 49)
-NH-GAVILRR(CO-)
(SEQ ID NO: 50)
-NH-DYKDDDDKG(CO-)
SEQ ID NO: 51)
-NH-VKVKVKVKVPPTKTEVKVKV(CO-)
(SEQ ID NO: 52)
-NH-AAAAAAD(CO-)
(SEQ ID NO: 53)
-NH-AAAAAAK(CO-)
(SEQ ID NO: 54)
-NH-DAAAAAA(CO-)
(SEQ ID NO: 55)
-NH-KAAAAAA(CO-)
(SEQ ID NO: 56)
-NH-YLGFFC(CO-)
(SEQ ID NO: 57)
-NH-KLILIK(CO-)
(SEQ ID NO: 58)
-NH-NYFNTKDRIYH(CO-)
(SEQ ID NO: 59)
-NH-YLGFFC(CO-)
(SEQ ID NO: 60)
-NH-FFFRRR(CO-)
(SEQ ID NO: 61)
-NH-FFFFRRRR(CO-)
(SEQ ID NO: 62)
-NH-FFYSV(CO-)
(SEQ ID NO: 63)
-NH-FFFGKG(CO-)
(SEQ ID NO: 64)
-NH-VVAAEE(CO-)
(SEQ ID NO: 65)
-NH-KLDLKLDLKLDL(CO-)
SEQ ID NO: 66)
-NH-YGAAKKAAKAAKKAAKAA(CO-)
(SEQ ID NO: 67)
-NH-KFDLKKDLKLDL(CO-)
(SEQ ID NO: 68)
-NH-FKFEFKFF(CO-)
(SEQ ID NO: 69)
-NH-FEFEFKFK(CO-)
(SEQ ID NO: 70),

6. A composition of claim 1, wherein:
R1 comprises a distal hydrophobic moiety (such as pyrene, naphthalene, adamantane, acetate, alkyl or fluorenylmethyloxycarbonate), which may be linked by amide bond on the amino side of the peptide.

7. A composition of claim 1, wherein:
R1 comprises a distal hydrophobic moiety, selected from pyrene, naphthalene, adamantane, acetate, alkyl and fluorenylmethyloxycarbonate, which is linked by amide bond on the amino side of the peptide.

8. A composition of claim 1, wherein:
R1 comprises a distal hydrophobic moiety selected from:

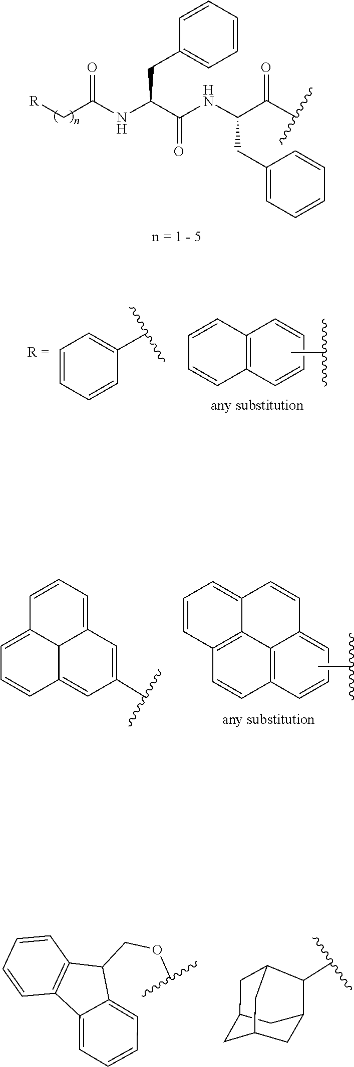

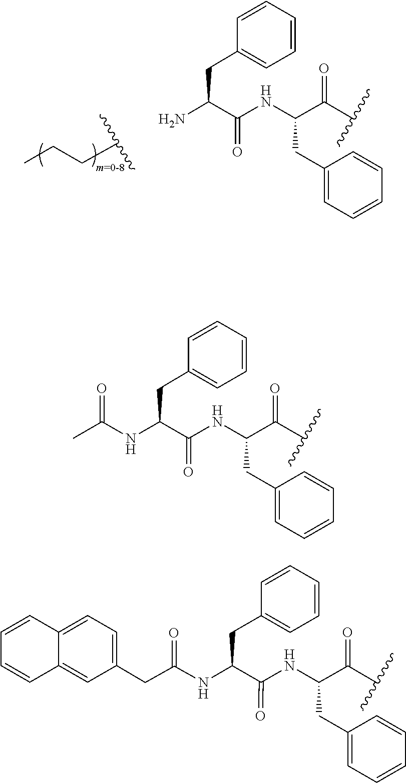

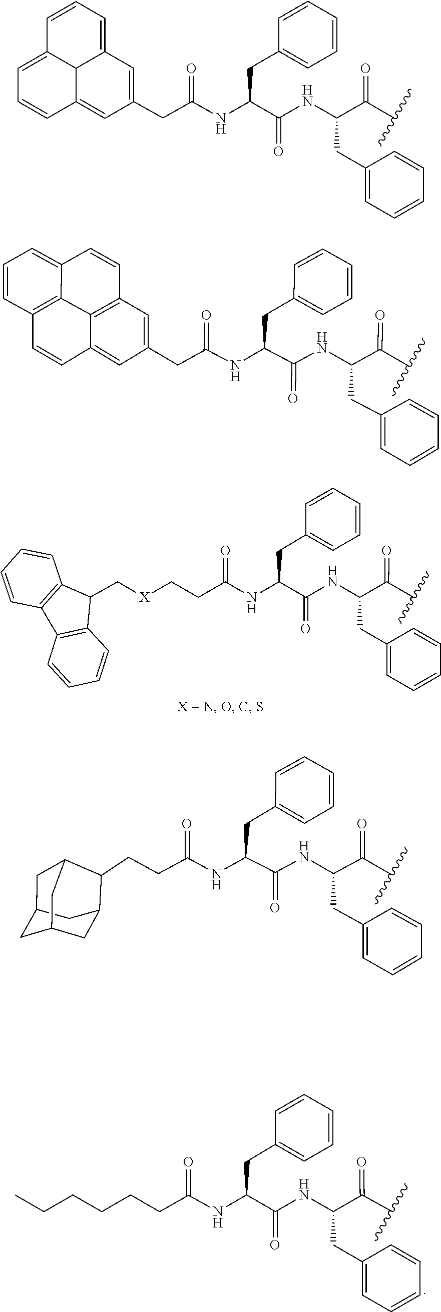

9. A composition of claim 1, wherein:
R2 comprises steroid selected from:

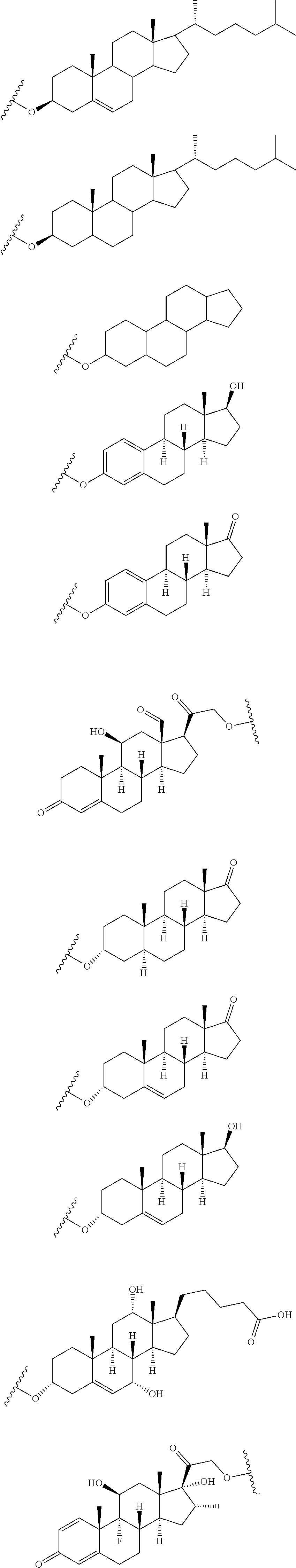

10. A composition of claim 1, wherein R2 comprises an alkyl chain: 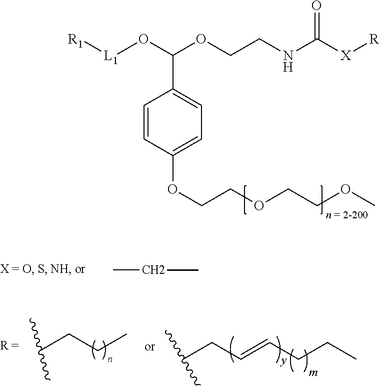
n=4-16; m=1-13; y=1-3, wherein the olefin(s) can be Z/E, and in any position(s) of the chain, such as:

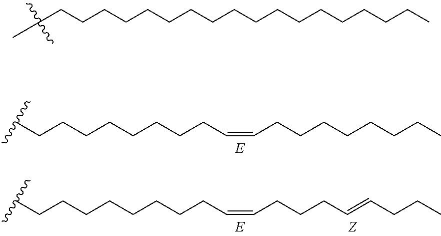 [SMILES: *CCCCCCCCCCCCCCCCCC ; *CCCCCCCC/C=C\CCCCCCCC ; *CCCCCCCC/C=C\CCC/C=C/CCC]

11. A composition of claim 1, wherein R2 comprises an alkyl chain, of structure: 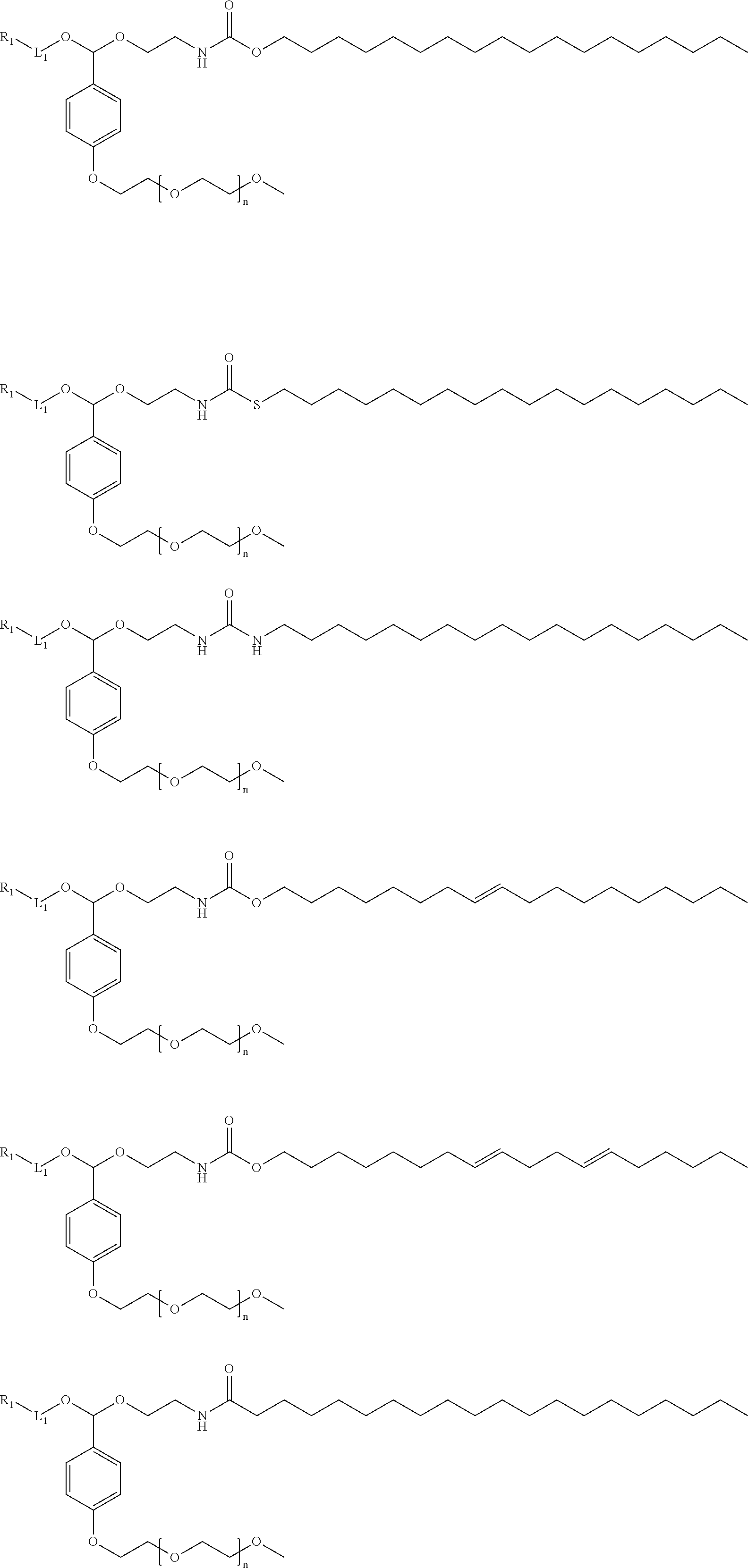
such as in:

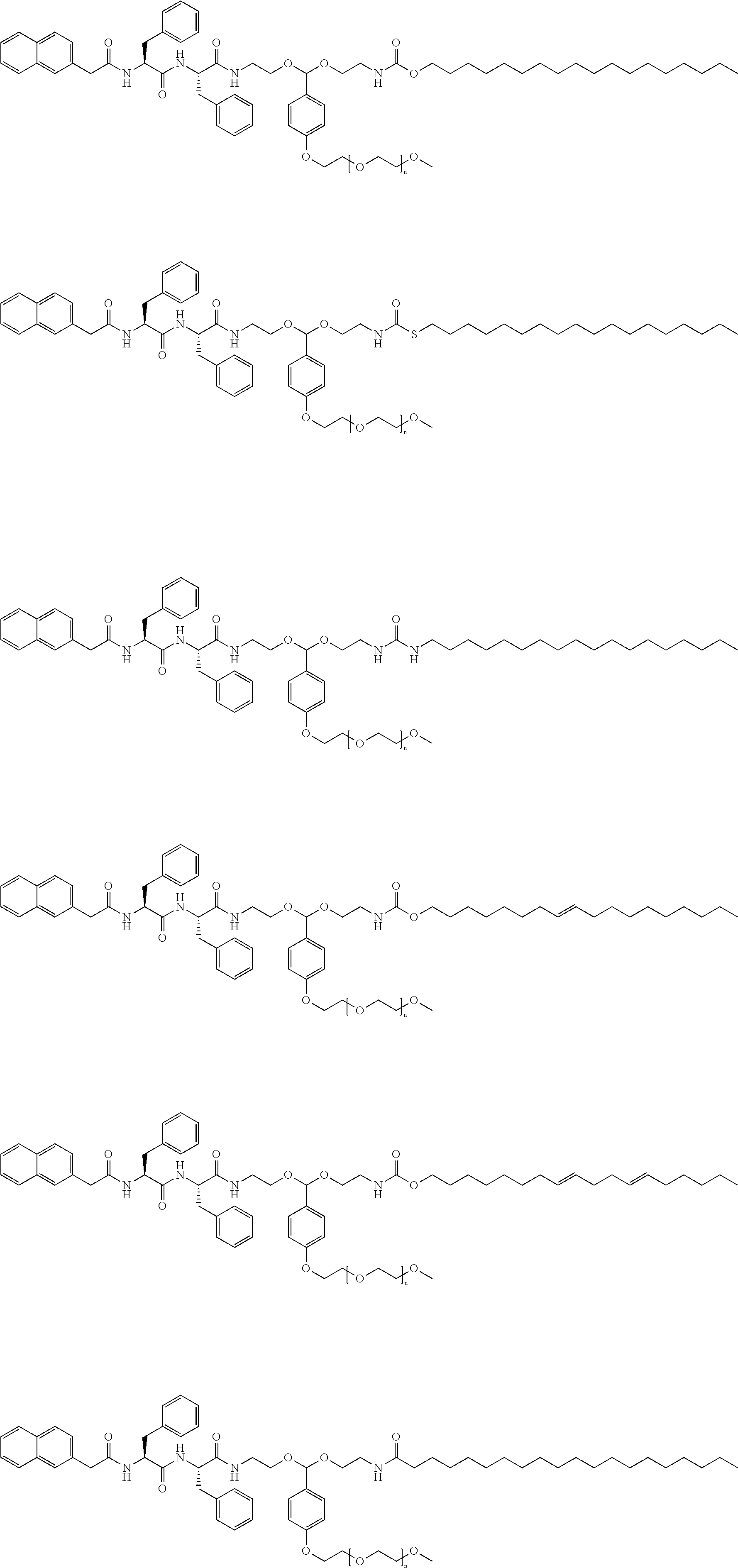

12. A composition of claim 1, wherein R2 comprises two alkyl chains: 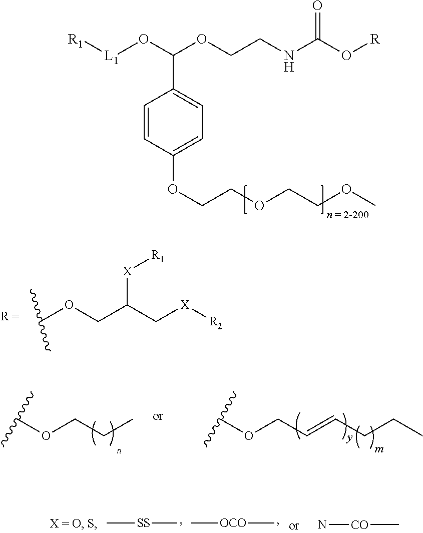
n=4-16; m=1-13; y=1-3, wherein the olefin(s) can be Z/E, and in any position(s) of the chain, such as:

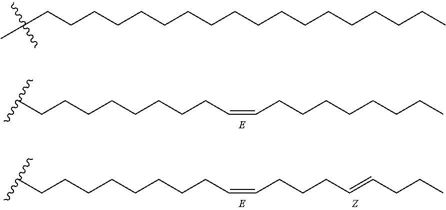 [SMILES: *CCCCCCCCCCCCCCCCCC ; *CCCCCCCC/C=C\CCCCCCCC ; *CCCCCCCC/C=C\CCC/C=C/CCC]

13. A composition of claim 1, wherein R2 comprises two alkyl chains, of structure: 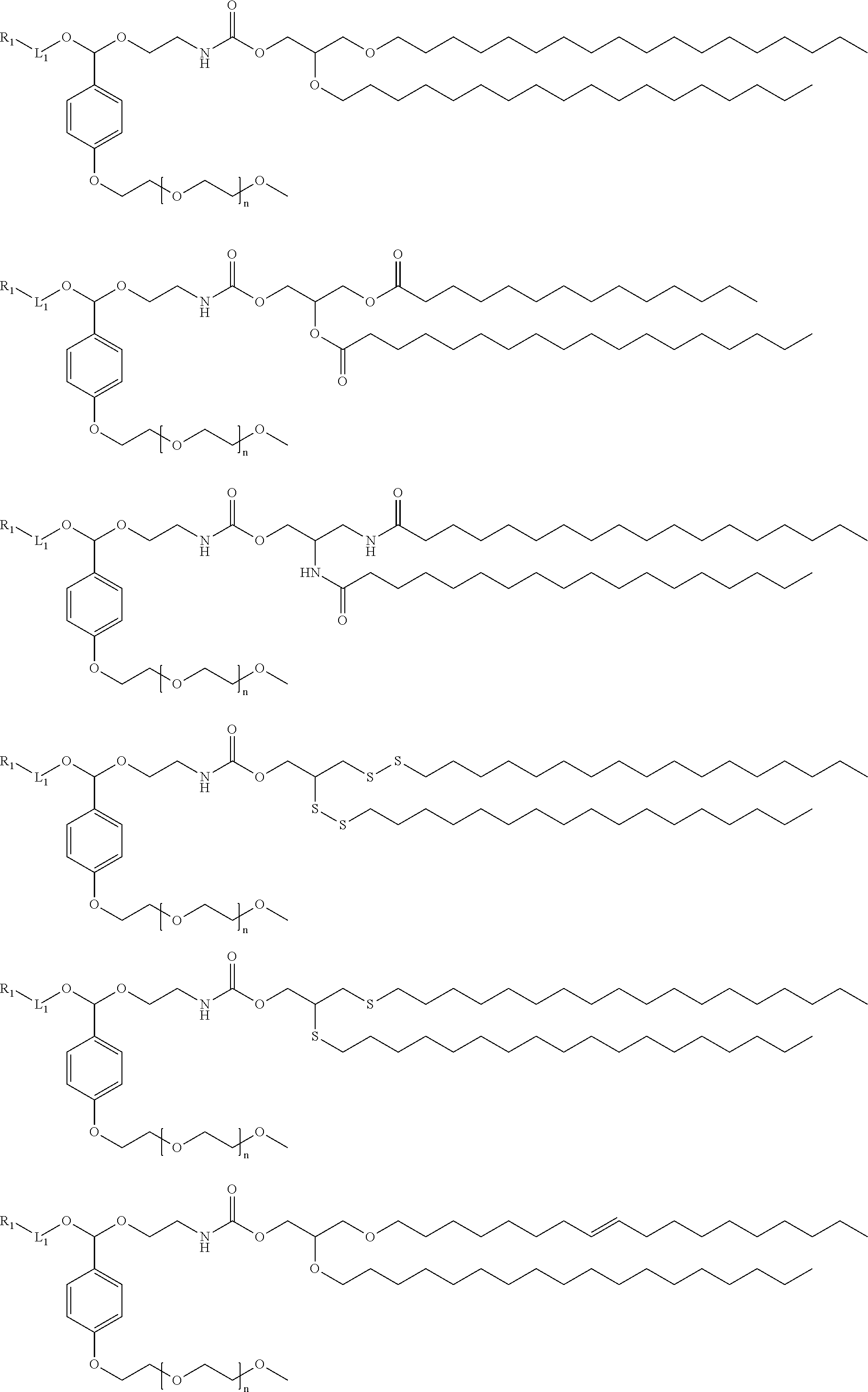 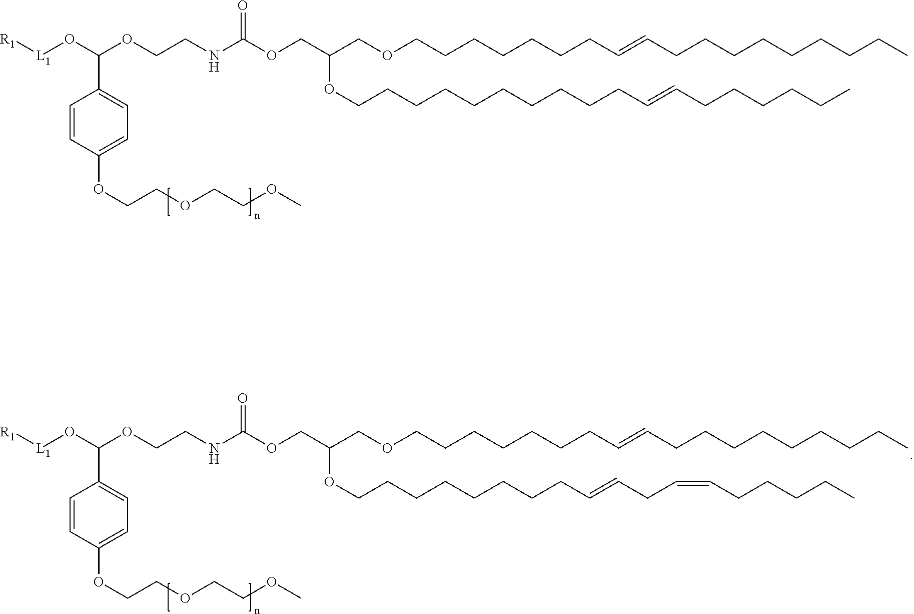
such as in:

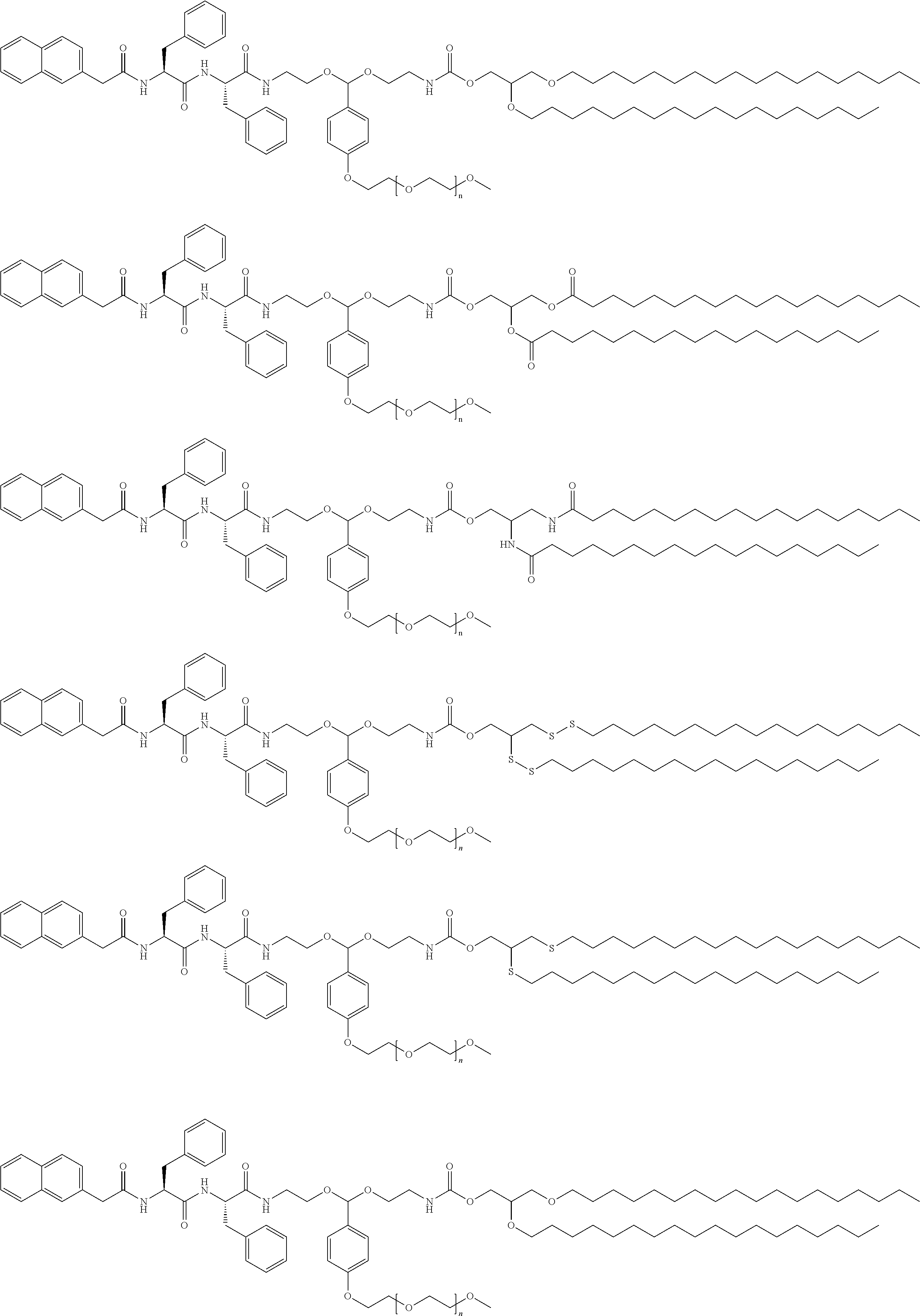

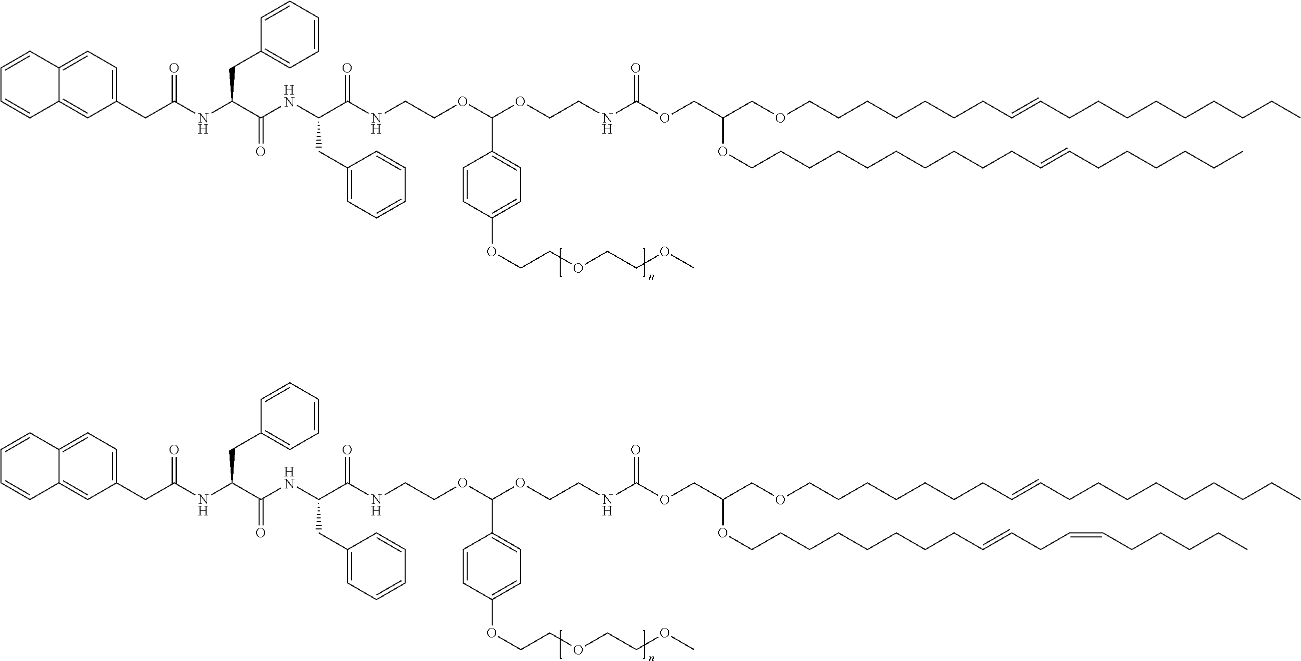

14. A composition of claim 1, wherein R2 comprises three alkyl chains: 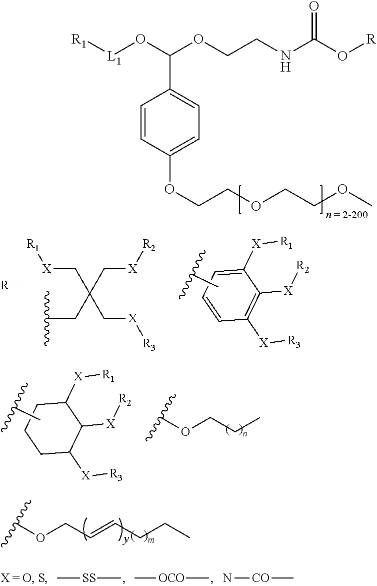
n=4-16; m=1-13; y=1-3, wherein the olefin(s) can be Z/E, and in any position(s) of the chain, such as:

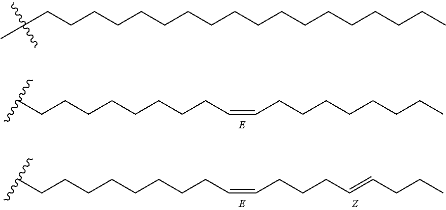 [SMILES: *CCCCCCCCCCCCCCCCCC ; *CCCCCCCC/C=C\CCCCCCCC ; *CCCCCCCC/C=C\CCC/C=C/CCC]

15. A composition of claim 1, wherein R2 comprises three alkyl chains, of structure: 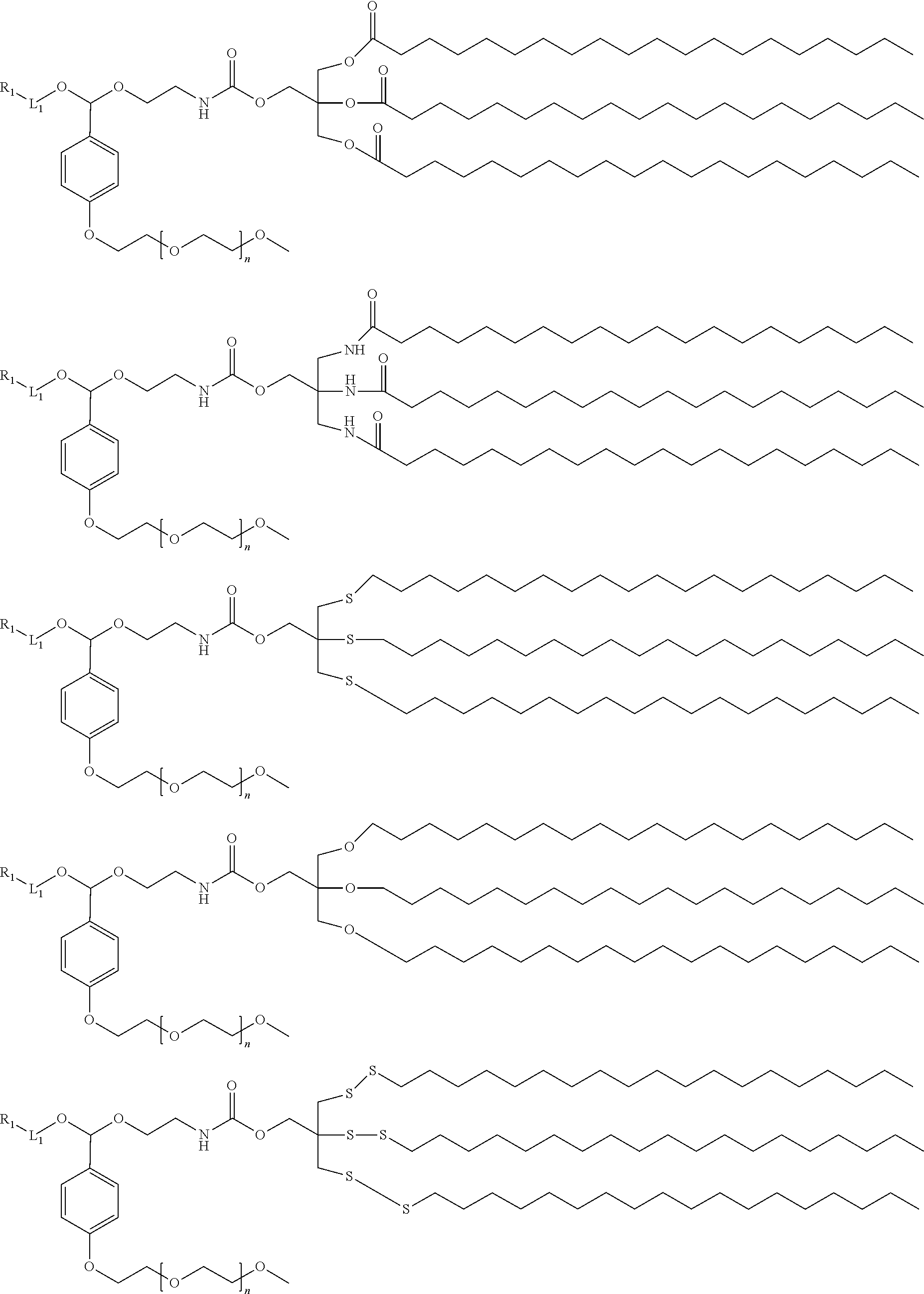 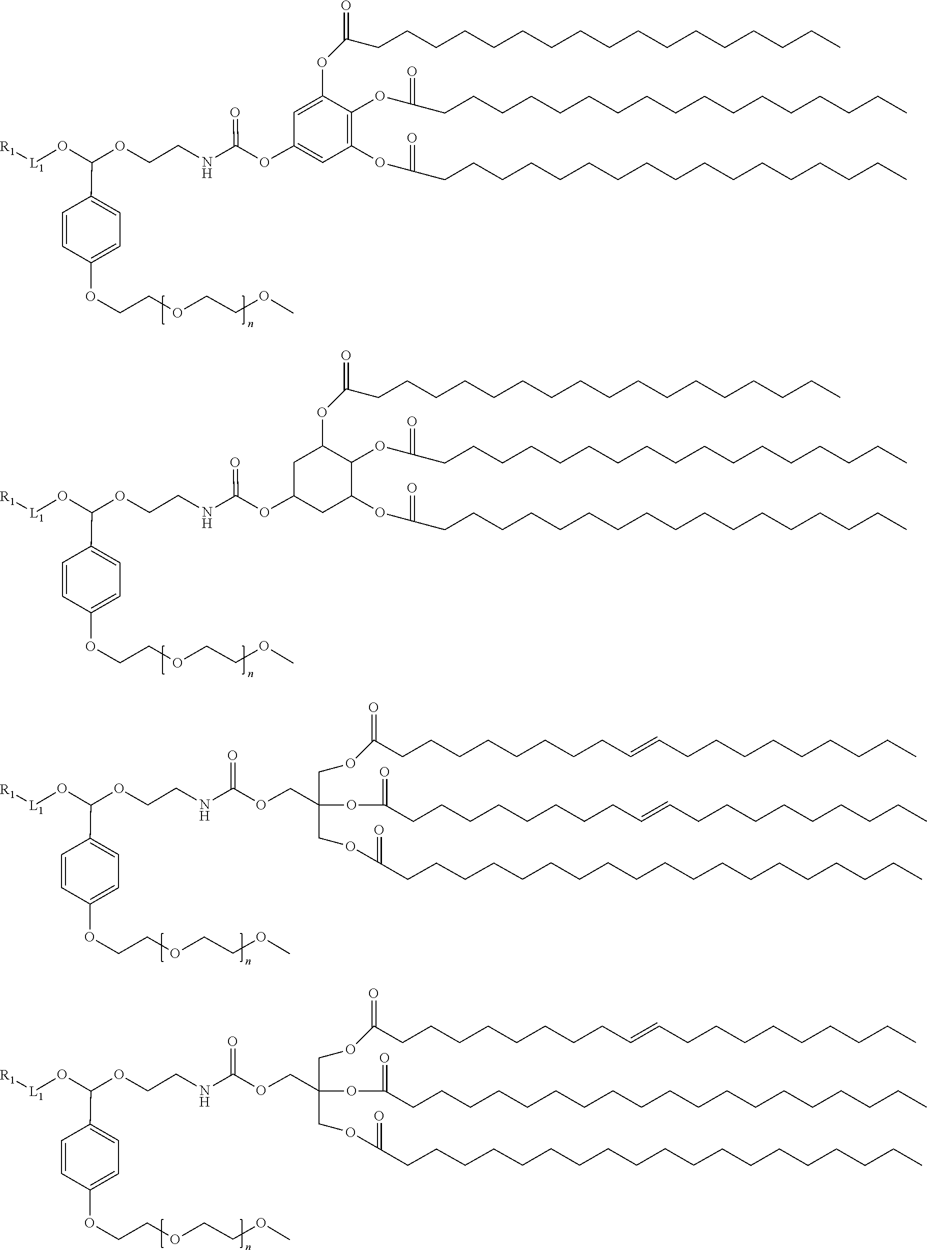 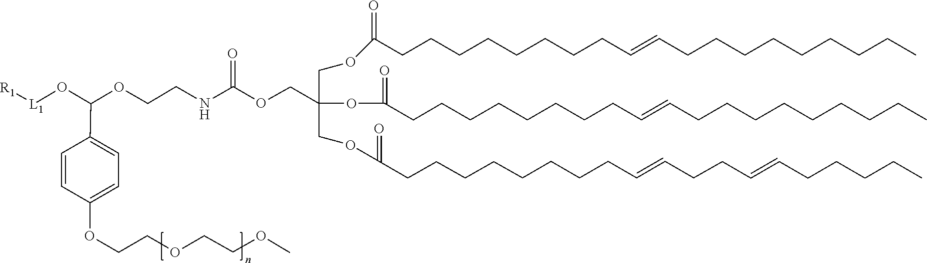
such as in:

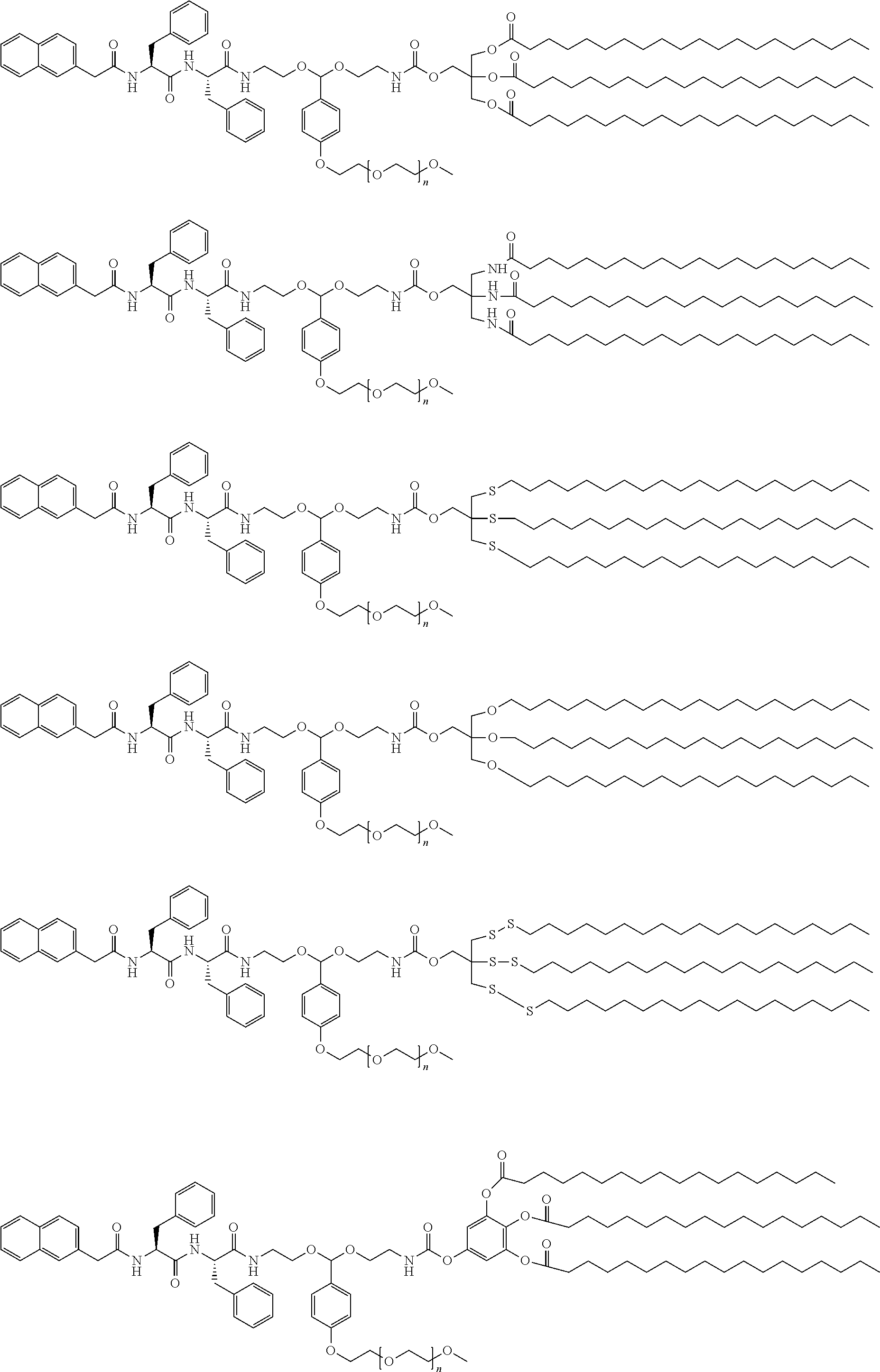

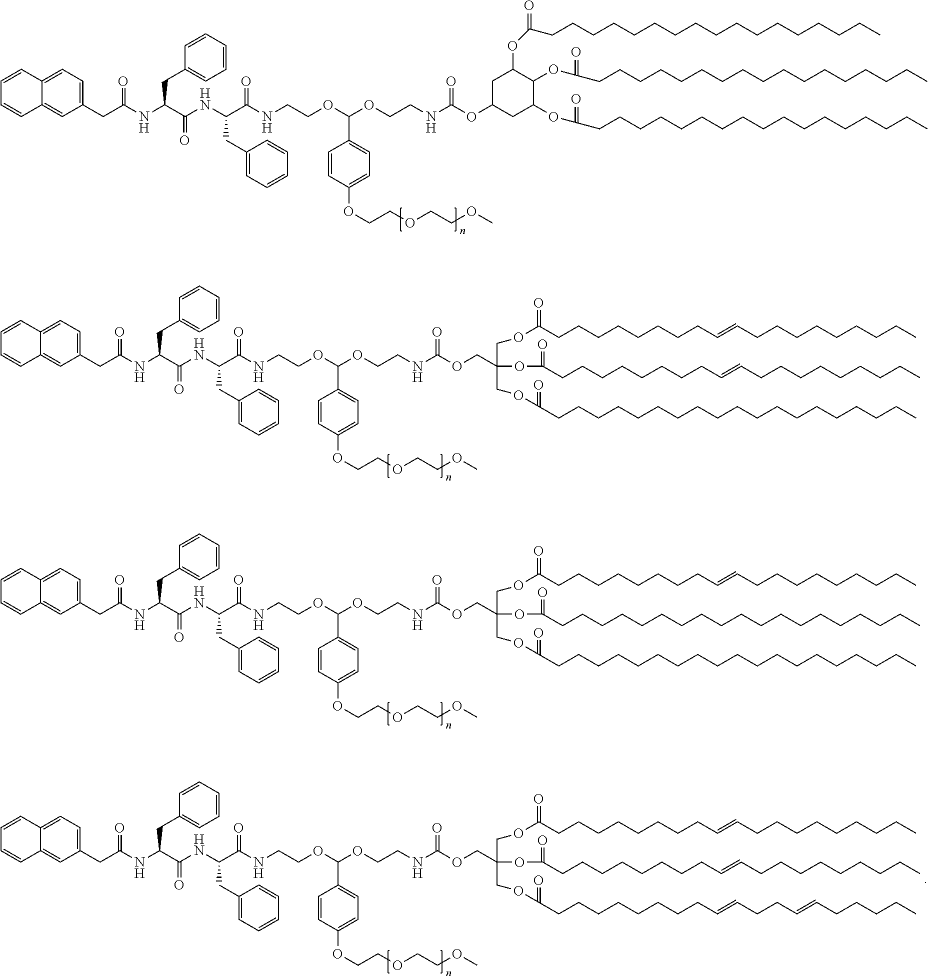

16. A composition of claim 1, wherein:
L1, L2 and L3 comprise linkers independently selected from optionally hetero-, optionally substituted linear C1-C12 alkyl.

17. A composition of claim 1, wherein:
L1, L2 and L3 comprise linkers independently selected from: 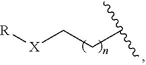
n=1-5; X=N, O, S; or 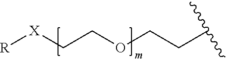
m=0-5, X=N, O, S.

18. A composition of claim 1, wherein:
L1 and L2 are —CH2CH2NH—R1 and —CH2CH2NHCO—R2, and L3 is a bond.

19. A composition of claim 1, wherein:
R3 comprises PEG of 500-5000 Da (e.g. 45-134 oxyethylene units).

20. A composition of claim 1, wherein:
L3 comprises a triazole.

21. A composition of claim 1, wherein:
L3 comprises a triazole click reaction product between an acetal alkyne and PEG-N3, e.g.: 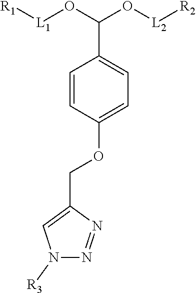
linked by amide bond with the peptide on the amino side.

22. A composition of claim 1, wherein L3 provides a direct link with R3 (e.g. a PEG), of structure: 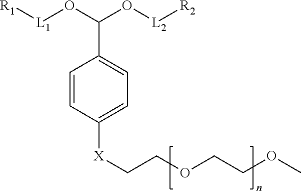
X=N, O, S, —OCO—, —SCO—, SS, —NCO—
n=2-200
such as in:

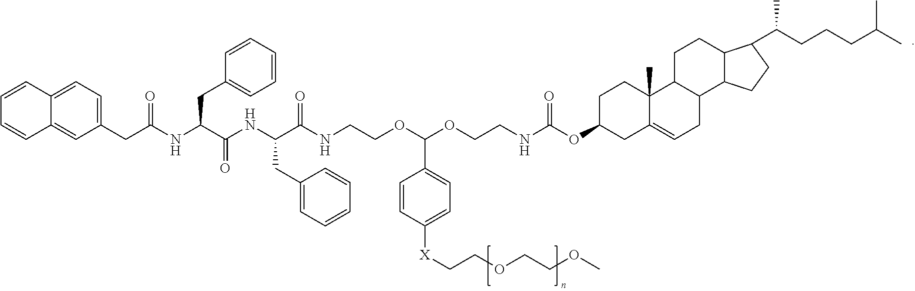

23. A composition of claim 1, wherein L3 provides a direct link with R3 (e.g. a PEG), of structure: 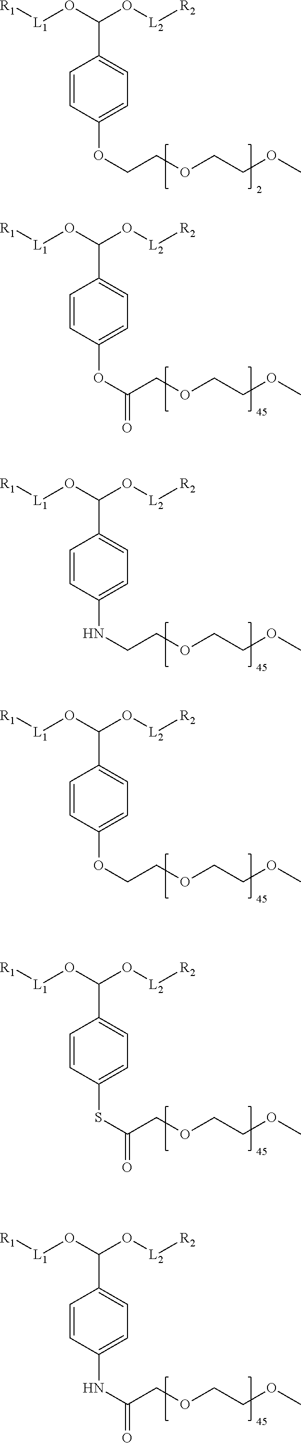 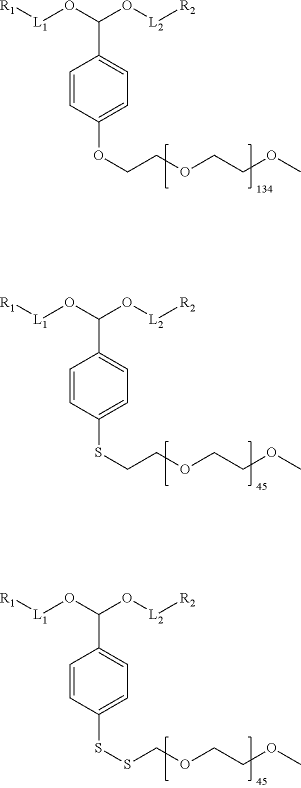
such as in:

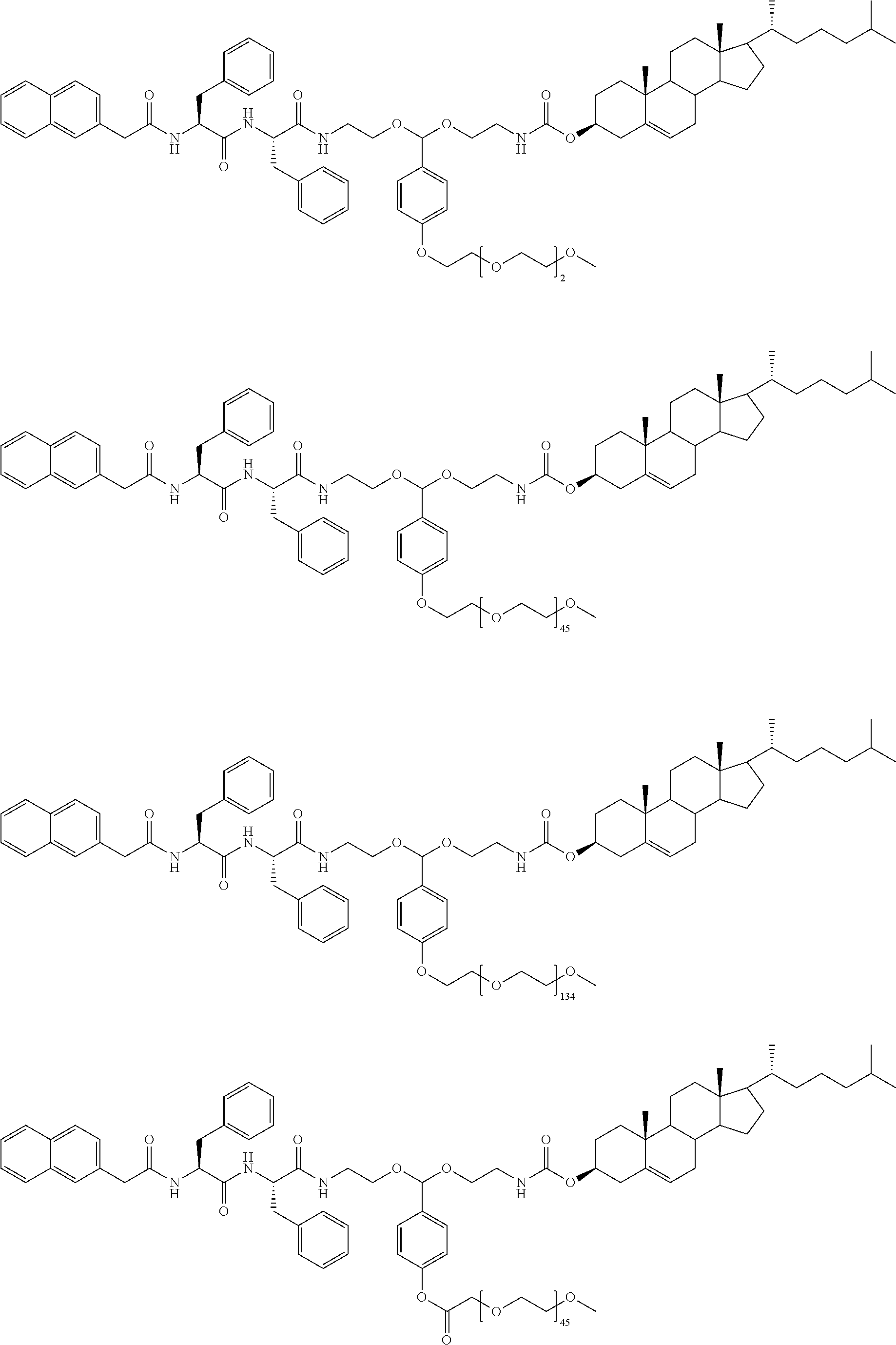

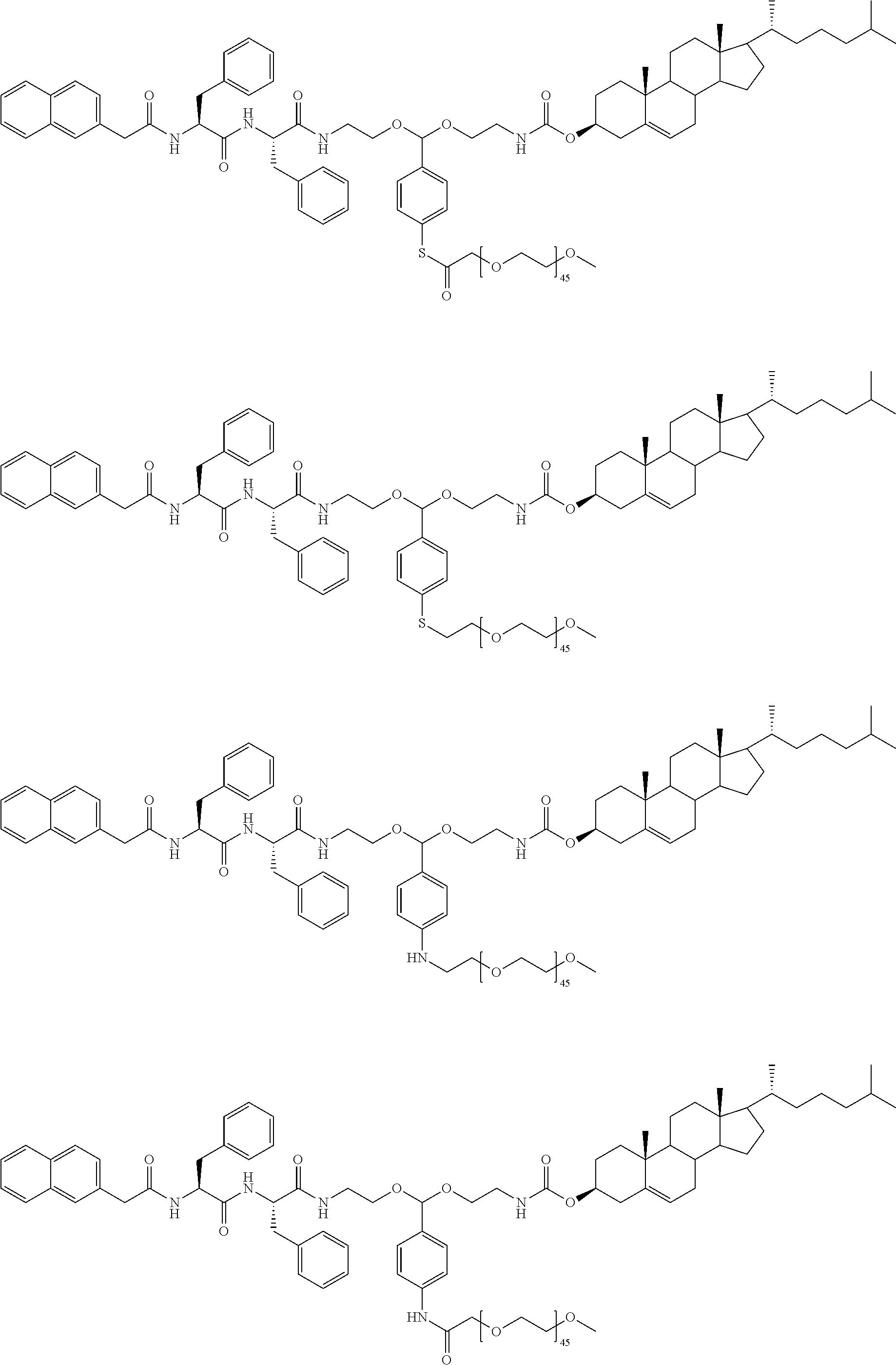

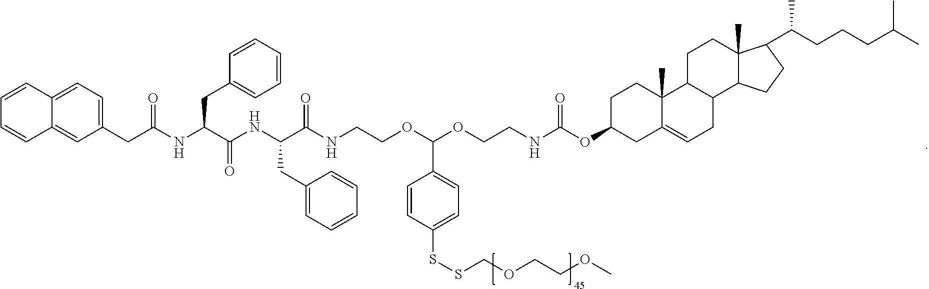

24. A composition of claim 1, wherein L3 provides a link with R3 (e.g. PEG) by click reaction, of structure: 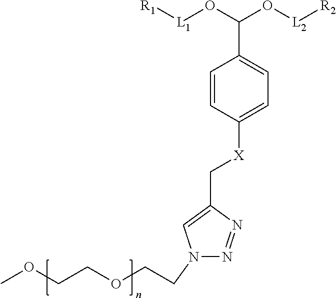
X=N, O, S, —OCO—, —SCO—, SS, —NCO—
n=2-200
such as in:

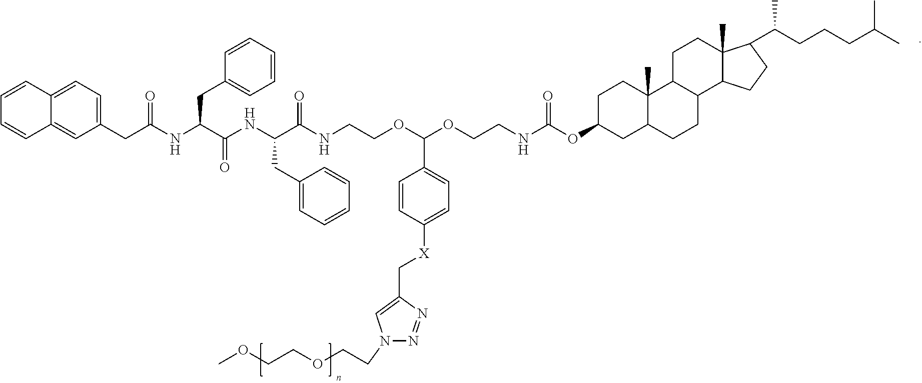

25. A composition of claim 1, wherein L3 provides a link with R3 (e.g. PEG) by click reaction, of structure: 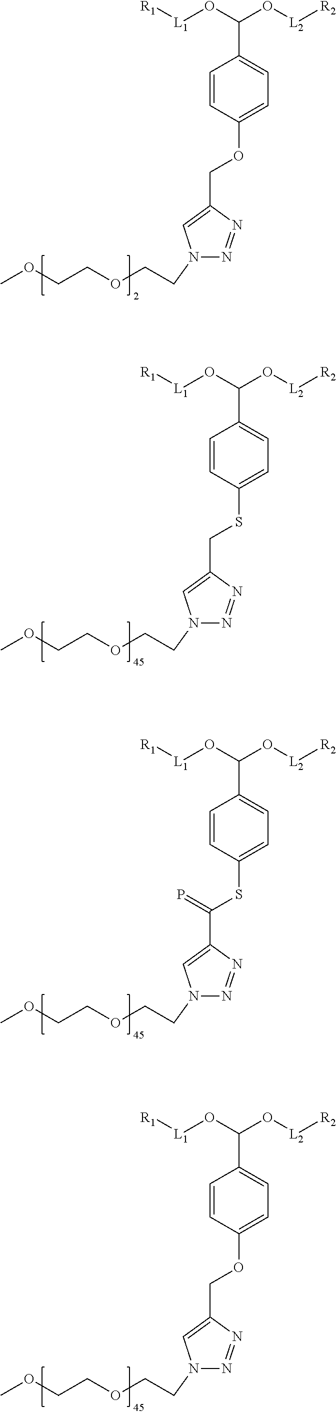 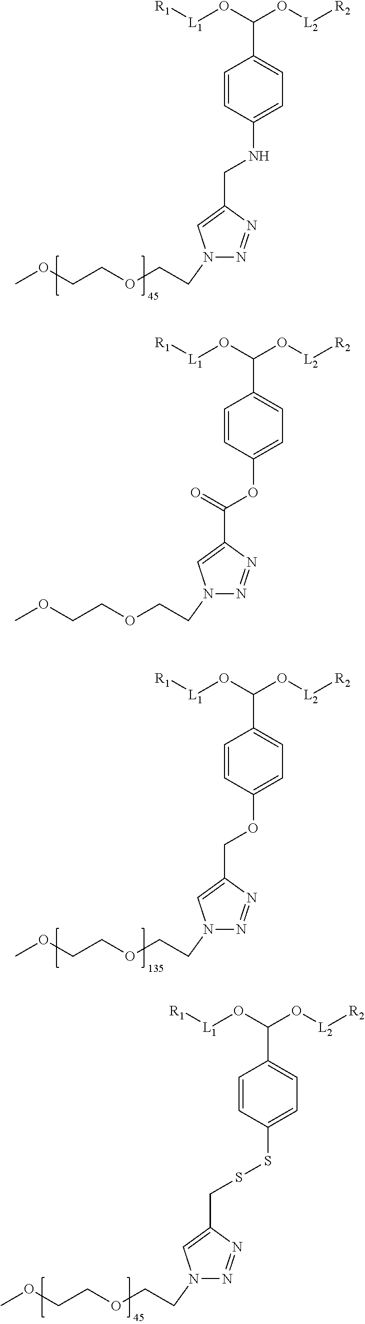 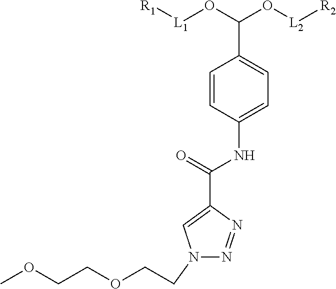
such as in:

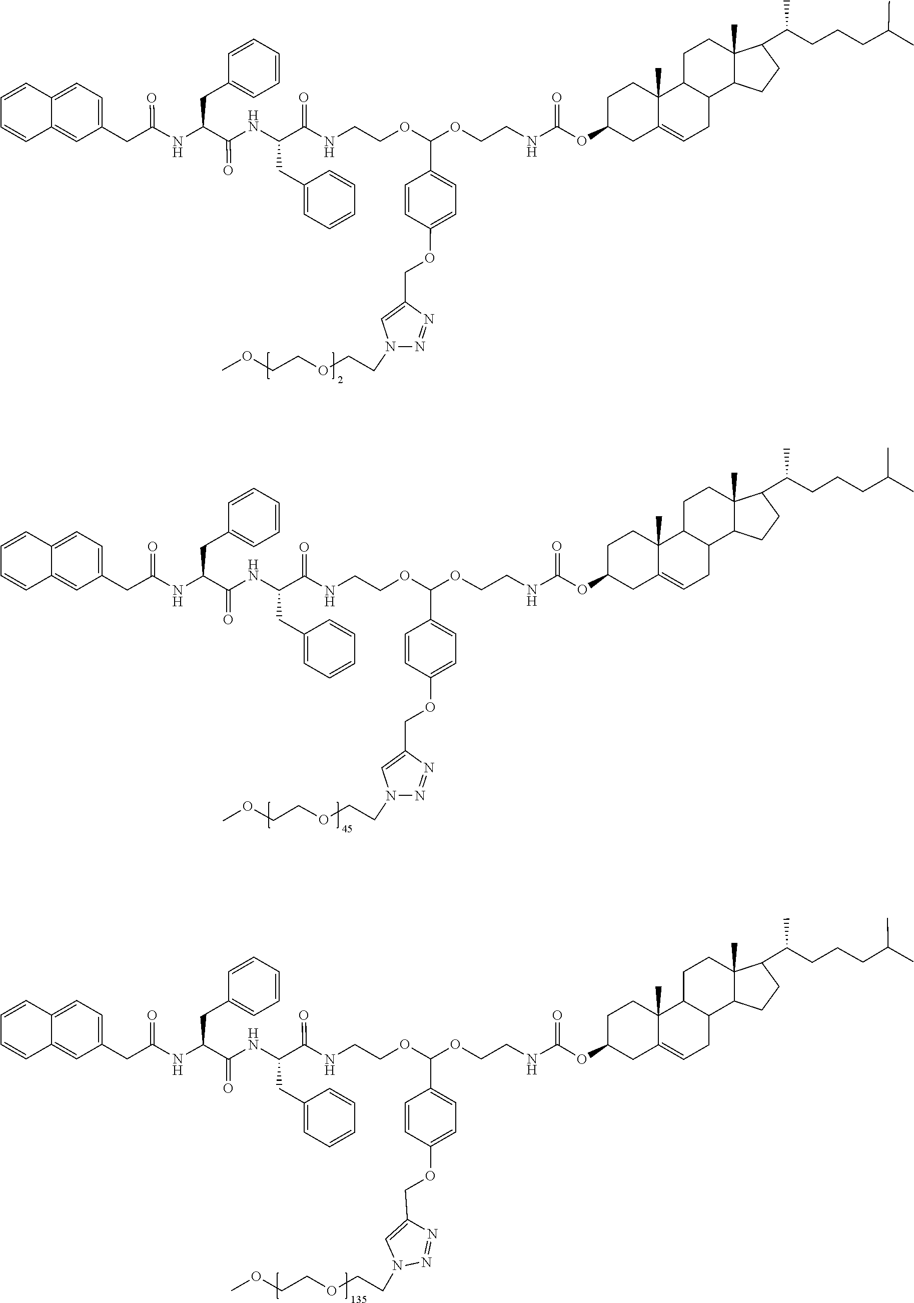

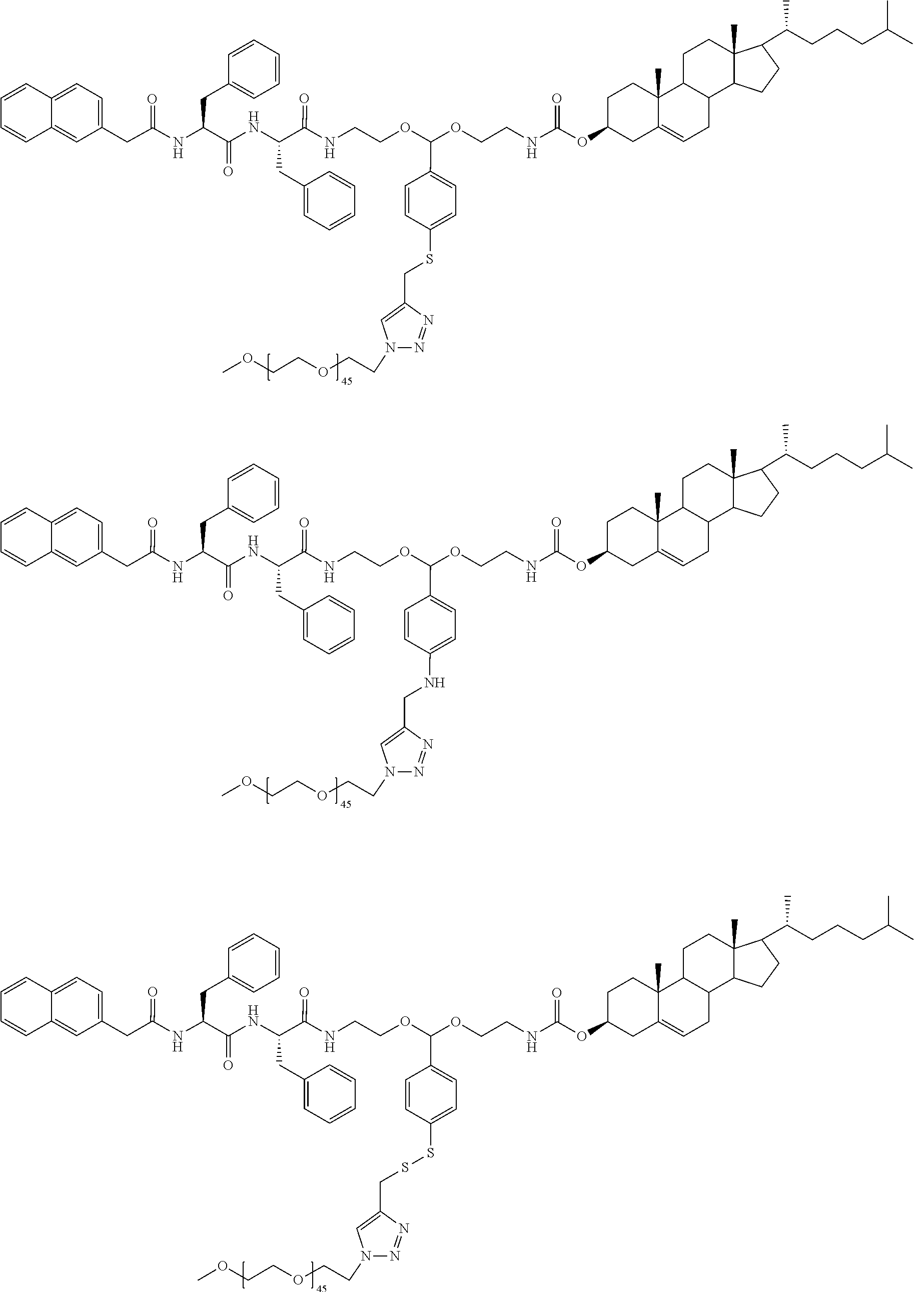

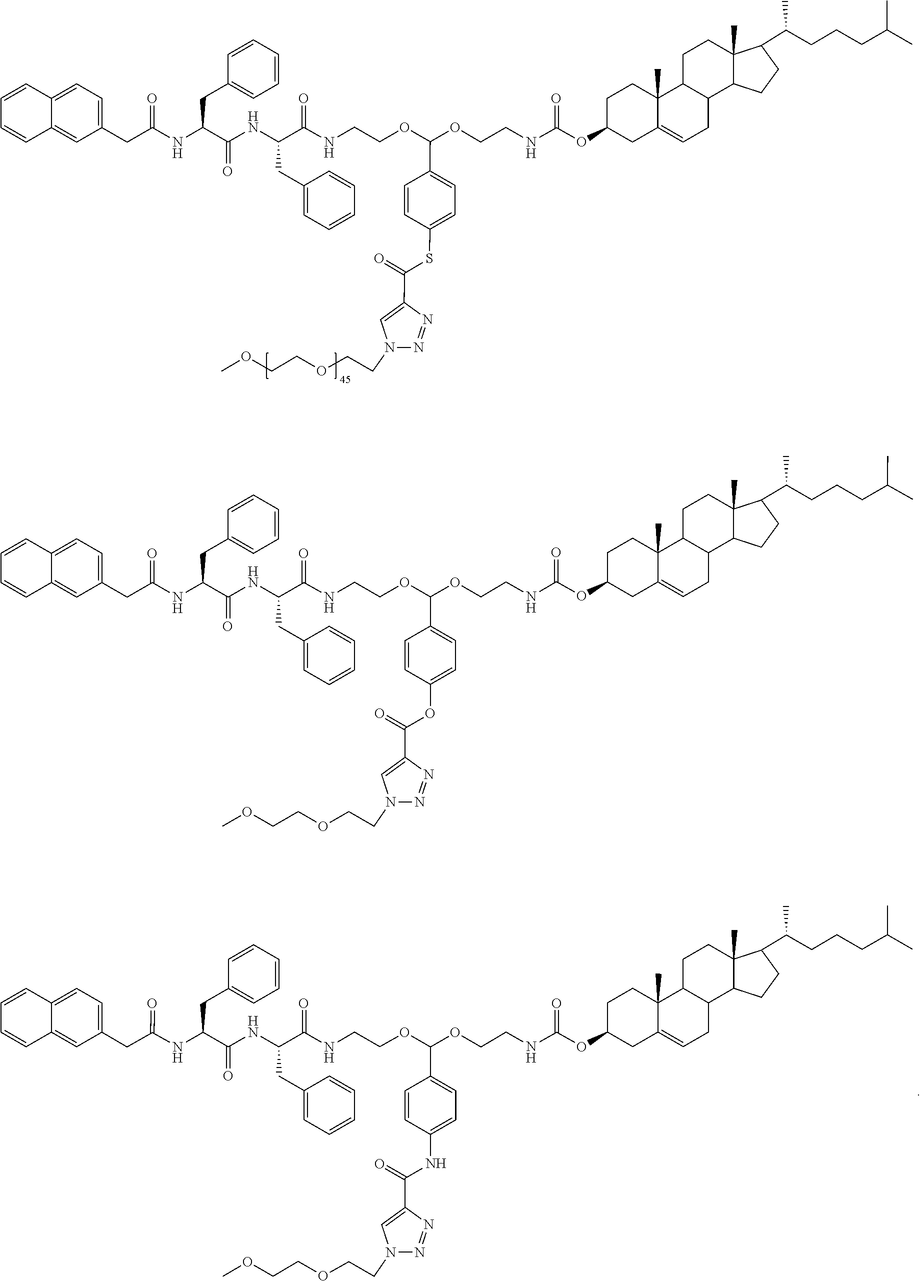

26. A composition of claim 1, comprising the structure of Pep-Chol-PEG-1k/2k:

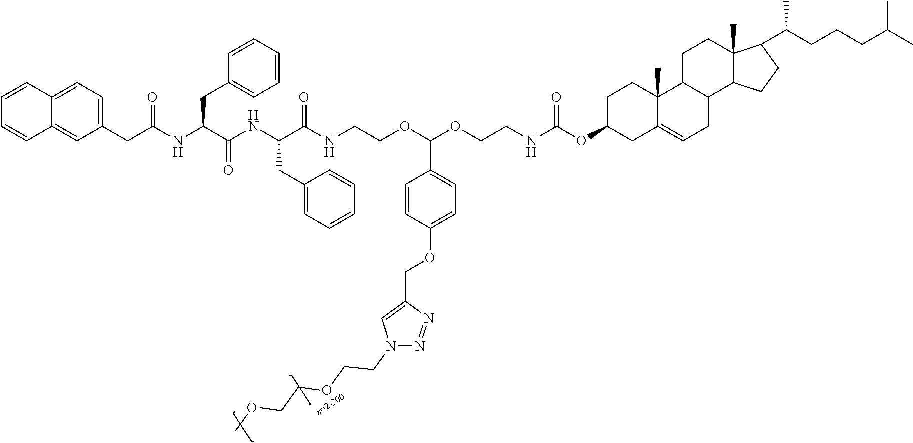

27. A composition of claim 1, formulated into solid lipid nanoparticles (SLNs) further comprising a nucleic acid, such as an RNA or DNA, encoding a therapeutic protein, vaccine antigen, or gene editing enzyme(s); or
a lipid nanoparticle (LNP) composition comprising a compound of claim 1, configured, for example, to deliver mRNA, plasma DNA, siRNA, for example, for vaccines, wherein for some of the mRNA, like Cas 9 mRNA may be combined with guide RNA, for cell editing and treating disease.

28. A method of making a compound of claim 1,
(a) comprising solid phase peptide synthesis; or
(b) comprising a triazole click reaction, wherein L3 comprises a triazole click reaction product between an acetal alkyne and PEG-N3, e.g.:

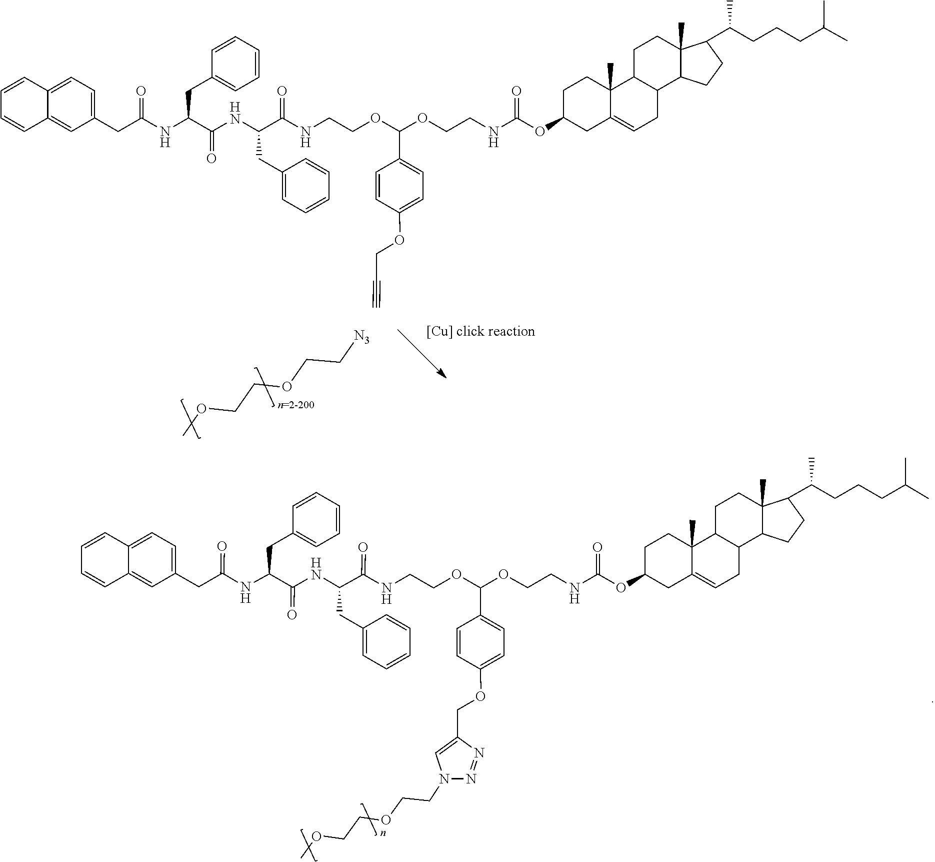

29. A method of using a compound of claim 1,
(a) comprising delivering the compounds in a lipid nanoparticle (LNP) composition comprising a compound herein, to transfect a tissue or organ, such as muscle, lung, spleen, liver and blood; or
comprising delivering the compounds in a lipid nanoparticle (LNP) composition comprising a compound herein configured as a vaccine or therapeutic, and preferably detecting a resultant intended, targeted effect, and preferably with enhanced effect, e.g. mRNA transfection efficiency attributable to use of the compound; or
wherein the acid cleavable linker is deployed to cage the self-assembling peptide, and releasing in an endosome (which can help/enhance mRNA transfection efficiency); or
wherein the compound is cleaved substantially according the mechanism: 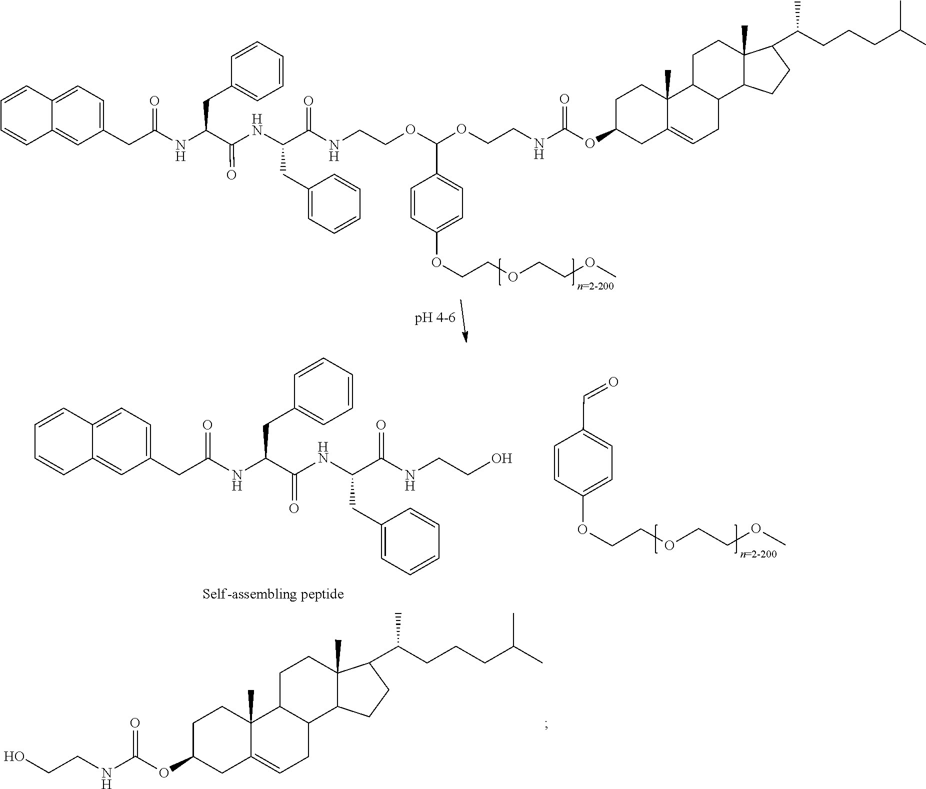
or wherein a resultant change in morphology is determined.

30. A solid-phase lipid synthesis method comprising synthesis of cationic, ionizable lipids via solid phase peptide synthesis, optionally comprising integration in an automated robotic system (ARS) of: (i) the solid phase lipid synthesis, (ii) initial cell screening, and (iii) animal organ or cell targeting.


SEQUENCE LISTING (76 sequences)

SEQ ID NO: 1 - protein, 4 aa, synthetic construct
FFKY

SEQ ID NO: 2 - protein, 4 aa, synthetic construct
FFFY

SEQ ID NO: 3 - protein, 4 aa, synthetic construct
KYFF

SEQ ID NO: 4 - protein, 4 aa, synthetic construct
TFFY

SEQ ID NO: 5 - protein, 4 aa, synthetic construct
FFYY

SEQ ID NO: 6 - protein, 4 aa, synthetic construct
FYFY

SEQ ID NO: 7 - protein, 4 aa, synthetic construct
YFYF

SEQ ID NO: 8 - protein, 4 aa, synthetic construct
FFYE

SEQ ID NO: 9 - protein, 4 aa, synthetic construct
FFYR

SEQ ID NO: 10 - protein, 4 aa, synthetic construct
GFFY

SEQ ID NO: 11 - protein, 4 aa, synthetic construct
LLLY

SEQ ID NO: 12 - protein, 4 aa, synthetic construct
GFYY

SEQ ID NO: 13 - protein, 4 aa, synthetic construct
FFSY

SEQ ID NO: 14 - protein, 4 aa, synthetic construct
FFYS

SEQ ID NO: 15 - protein, 5 aa, synthetic construct
FFAGL

SEQ ID NO: 16 - protein, 4 aa, synthetic construct
FFAG

SEQ ID NO: 17 - protein, 4 aa, synthetic construct
FFYD

SEQ ID NO: 18 - protein, 4 aa, synthetic construct
FFEY

SEQ ID NO: 19 - protein, 5 aa, synthetic construct
LLLLY

SEQ ID NO: 20 - protein, 5 aa, synthetic construct
LLYLL

SEQ ID NO: 21 - protein, 5 aa, synthetic construct
FFGGY

SEQ ID NO: 22 - protein, 6 aa, synthetic construct
GFFYSV

SEQ ID NO: 23 - protein, 5 aa, synthetic construct
GFFYG

SEQ ID NO: 24 - protein, 5 aa, synthetic construct
FFGEY

SEQ ID NO: 25 - protein, 5 aa, synthetic construct
FFGDY

SEQ ID NO: 26 - protein, 5 aa, synthetic construct
FFEYI

SEQ ID NO: 27 - protein, 6 aa, synthetic construct
FFFFCG

SEQ ID NO: 28 - protein, 6 aa, synthetic construct
CEYFFG

SEQ ID NO: 29 - protein, 7 aa, synthetic construct
FFYGGAA

SEQ ID NO: 30 - protein, 7 aa, synthetic construct
GFFYGHY

SEQ ID NO: 31 - skipped

SEQ ID NO: 32 - protein, 7 aa, synthetic construct
FFAGLDD

SEQ ID NO: 33 - protein, 7 aa, synthetic construct
FFYGGVV

SEQ ID NO: 34 - skipped

SEQ ID NO: 35 - protein, 6 aa, synthetic construct
LYYYYL

SEQ ID NO: 36 - protein, 4 aa, synthetic construct
VVVD

SEQ ID NO: 37 - protein, 4 aa, synthetic construct
VVVK

SEQ ID NO: 38 - protein, 5 aa, synthetic construct
VVVVD

SEQ ID NO: 39 - protein, 6 aa, synthetic construct
VVVVDD

SEQ ID NO: 40 - protein, 6 aa, synthetic construct
VVVVWD

SEQ ID NO: 41 - protein, 7 aa, synthetic construct
VVVVWDD

SEQ ID NO: 42 - protein, 7 aa, synthetic construct
IIIIWDD

SEQ ID NO: 43 - protein, 7 aa, synthetic construct
LLLLWDD

SEQ ID NO: 44 - protein, 11 aa, synthetic construct
QQKFQFQFEQQ

SEQ ID NO: 45 - protein, 4 aa, synthetic construct
IIID

SEQ ID NO: 46 - protein, 4 aa, synthetic construct
IIIK

SEQ ID NO: 47 - protein, 4 aa, synthetic construct
LLLD

SEQ ID NO: 48 - protein, 4 aa, synthetic construct
LLLK

SEQ ID NO: 49 - protein, 7 aa, synthetic construct
GAVILEE

SEQ ID NO: 50 - protein, 7 aa, synthetic construct
GAVILRR

SEQ ID NO: 51 - protein, 9 aa, synthetic construct
DYKDDDDKG

SEQ ID NO: 52 - protein, 20 aa, synthetic construct
VKVKVKVKVPPTKTEVKVKV

SEQ ID NO: 53 - protein, 7 aa, synthetic construct
AAAAAAD

SEQ ID NO: 54 - protein, 7 aa, synthetic construct
AAAAAAK

SEQ ID NO: 55 - protein, 7 aa, synthetic construct
DAAAAAA

SEQ ID NO: 56 - protein, 7 aa, synthetic construct
KAAAAAA

SEQ ID NO: 57 - protein, 6 aa, synthetic construct
YLGFFC

SEQ ID NO: 58 - protein, 6 aa, synthetic construct
KLILIK

SEQ ID NO: 59 - skipped

SEQ ID NO: 60 - protein, 6 aa, synthetic construct
YLGFFC

SEQ ID NO: 61 - protein, 6 aa, synthetic construct
FFFRRR

SEQ ID NO: 62 - protein, 8 aa, synthetic construct
FFFFRRRR

SEQ ID NO: 63 - protein, 5 aa, synthetic construct
FFYSV

SEQ ID NO: 64 - protein, 6 aa, synthetic construct
FFFGKG

SEQ ID NO: 65 - skipped

SEQ ID NO: 66 - protein, 12 aa, synthetic construct
KLDLKLDLKLDL

SEQ ID NO: 67 - protein, 18 aa, synthetic construct
YGAAKKAAKAAKKAAKAA

SEQ ID NO: 68 - protein, 12 aa, synthetic construct
KFDLKKDLKLDL

SEQ ID NO: 69 - protein, 8 aa, synthetic construct
FKFEFKFF

SEQ ID NO: 70 - protein, 8 aa, synthetic construct
FEFEFKFK

SEQ ID NO: 71 - protein, 7 aa, synthetic construct
GDDDDDG

SEQ ID NO: 72 - protein, 7 aa, synthetic construct
GEEEEEG

SEQ ID NO: 73 - protein, 4 aa, synthetic construct
RRRR

SEQ ID NO: 74 - protein, 7 aa, synthetic construct
IRQRRRR

SEQ ID NO: 75 - protein, 13 aa, synthetic construct
GGRKKRRQRRRPQ

SEQ ID NO: 76 - protein, 6 aa, synthetic construct
DDDDDG